(19) United States
(12) Patent Application Publication
(10) Pub. No.: US 2022/0306679 A1
(43) Pub. Date: Sep. 29, 2022

(54) LIGAND-2'-MODIFIED NUCLEIC ACIDS, SYNTHESIS THEREOF AND INTERMEDIATE COMPOUNDS THEREOF

(71) Applicant: Dicerna Pharmaceuticals, Inc., Lexington, MA (US)

(72) Inventors: Weimin WANG, Lexington, MA (US); Naim NAZEF, Lexington, MA (US)

(21) Appl. No.: 17/753,336

(22) PCT Filed: Aug. 28, 2020
(86) PCT No.: PCT/US2020/048313

§ 371 Date: Feb. 28, 2022

Related U.S. Application Data
(60) Provisional application No. 62/894,071, filed on Aug. 30, 2019.

(51) Int. Cl. C07H 21/02; C07H 19/167; C07H 19/067; C07H 1/00

(52) U.S. Cl. CPC C07H 21/02; C07H 19/167; C07H 19/067; C07H 1/00

(57) ABSTRACT

The present invention relates to methods for synthesizing compounds useful as potent and stable RNA interference agents, derivatives thereof, and intermediates thereto.

### CROSS-REFERENCE TO RELATED APPLICATION

This application claims the benefit under 35 U.S.C. § 119(e) of U.S. Provisional Application No. 62/894,071, filed Aug. 30, 2019, the content of which is incorporated by reference herein in its entirety.

### TECHNICAL FIELD OF THE INVENTION

The present invention relates to method for synthesizing compounds useful as potent and stable RNA interference agents, derivatives thereof, and intermediates thereto.

### BACKGROUND OF THE INVENTION

Double-stranded RNA (dsRNA) agents possessing strand lengths of 25 to 35 nucleotides have been described as effective inhibitors of target gene expression in mammalian cells (Rossi et al., U.S. Patent Publication Nos. 2005/0244858 and 2005/0277610). dsRNA agents of such length are believed to be processed by the Dicer enzyme of the RNA interference (RNAi) pathway, leading such agents to be termed “Dicer substrate siRNA” (“DsiRNA”) agents. Certain modified structures of DsiRNA agents were previously described (Rossi et al., U.S. Patent Publication No. 2007/0265220).

### DETAILED DESCRIPTION OF CERTAIN EMBODIMENTS

The methods and intermediates of the present disclosure are useful for preparing various analogues of compounds as described in, e.g. Brown et al., U.S. Patent Publication No. 2017/0305956, the entirety of which is herein incorporated by reference. The compounds provided herein are useful as pharmaceutical agents for the treatment of disease. In certain embodiments, a compound of formula A is generally prepared by the assembly of three fragments F-1, F-2, and F-3 as shown by Scheme 1 set forth below:

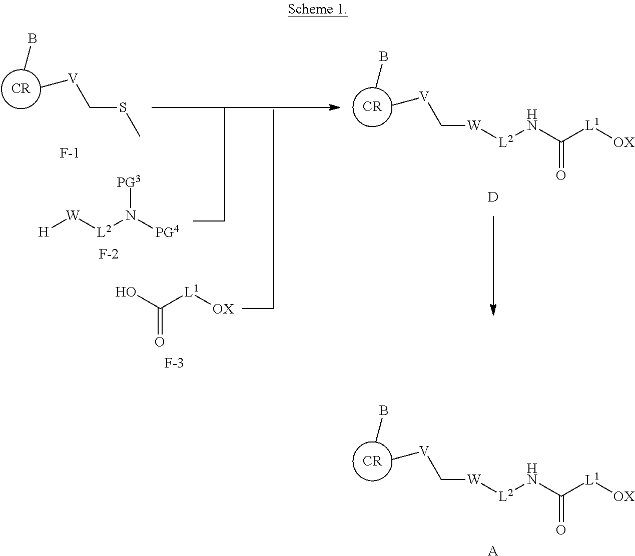

In Scheme 1 above, each of

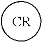

PG³, PG⁴, B, L¹, L², V, W, and X is as defined and in classes and subclasses as described herein.

In certain embodiments,

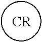

is

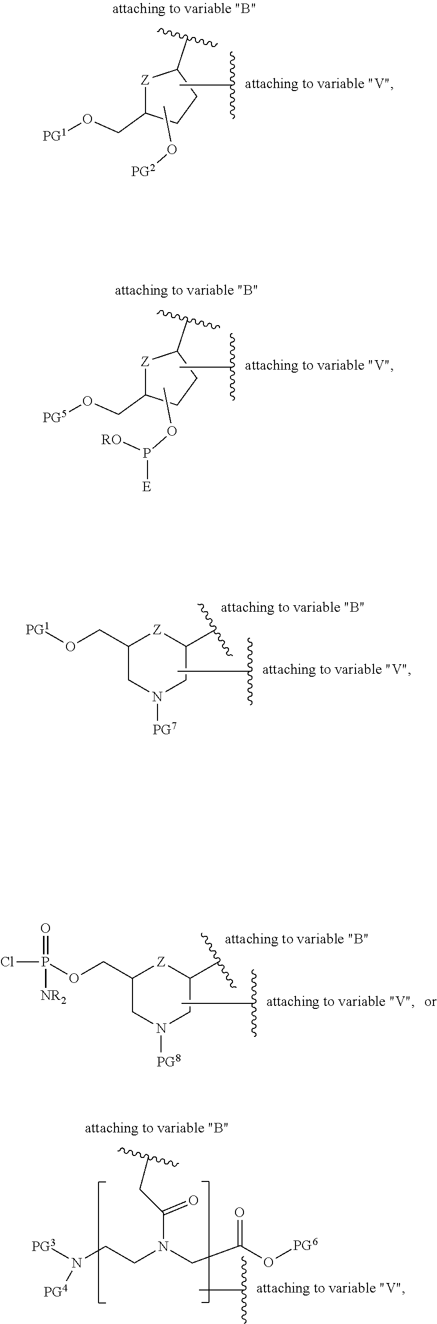

where PG¹, PG², PG³, PG⁴, PG⁵, PG⁶, PG⁷, PG⁸, E, R, and Z is as further defined and in classes and subclasses as described herein.

According to one embodiment, a compound of formula A-a is generally prepared by the assembly of three fragments F-1-a, F-2, and F-3 as shown by Scheme 2 set forth below

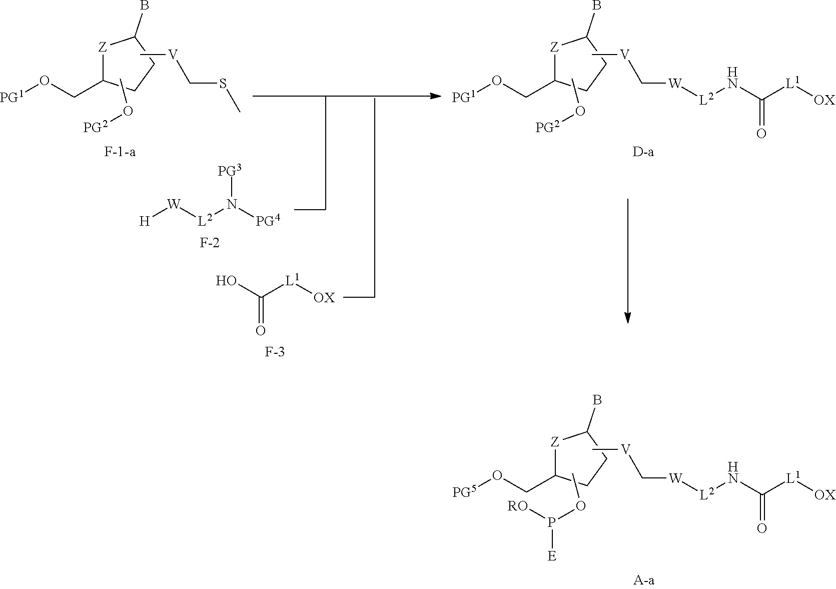

In Scheme 2 above, each of PG¹, PG², PG³, PG⁴, PG⁵, B, E, L¹, L², R, V, W, X, and Z is as defined and in classes and subclasses as described herein.

In some embodiments, Z is —O—.

1. Fragment Compound F-1-a

According to one embodiment, a fragment compound of formula F-1-a is generally prepared according to Scheme A set forth below:

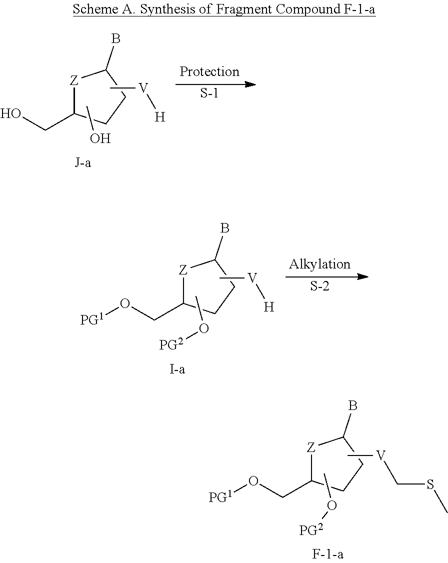

In Scheme A above, each of PG¹, PG², B, V, and Z is as defined and in classes and subclasses as described herein.

At step S-1, a compound of formula J-a is protected to afford a compound of formula I-a. In certain embodiments, the protecting groups PG¹ and PG² used for the protection of the hydroxyl groups of a compound of formula J-a include suitable hydroxyl protecting groups.

Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

In certain embodiments, the PG¹ and PG² groups of formula I-a are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula J-a and 1,3-dichloro-1,1,3,3-tetraisopropyldisiloxane under basic conditions.

At step S-2, a compound of formula I-a is alkylated with a mixture of DMSO and acetic anhydride under acidic conditions. In certain embodiments, when —V—H is a hydroxyl group, the mixture of DMSO and acetic anhydride in the presence of acetic acid forms (methylthio)methyl acetate in situ via the Pummerer rearrangement which then reacts with the hydroxyl group of the compound of formula I-a to provide a monothioacetal functionalized fragment compound of formula F-1-a.

2. Fragment Compound F-3

According to one embodiment, a fragment compound of formula F-3 is generally prepared according to Scheme B set forth below:

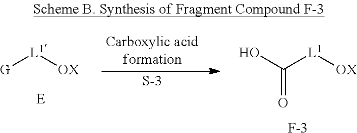

In Scheme B above, each of L¹, L^{1′}, G, and X is as defined and in classes and subclasses as described herein.

At step S-3, a compound of formula E is treated under conditions suitable to form a fragment compound of formula F-3, wherein G is a carboxylic acid having a suitable carboxylate protecting group or a functional group that can be reacted to form a carboxylic acid.

Suitable carboxylate protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable carboxylate protecting groups include, but are not limited to, substituted C₁₋₆ aliphatic esters, optionally substituted aryl esters, silyl esters, activated esters (e.g., derivatives of nitrophenol, pentafluorophenol, N-hydroxylsuccinimide, hydroxybenzotriazole, etc.), orthoesters, and the like. Examples of such ester groups include methyl, ethyl, propyl, isopropyl, butyl, isobutyl, tert-butyl, benzyl, and phenyl wherein each group is optionally substituted. Functional groups that can be reacted to form carboxylic acids include, but are not limited to, amides, hydrazides, oxazolines, alkyl halides, alkenes, alkynes, and nitriles. In certain embodiments, G is an alkenyl group.

In some embodiments, when G of a compound of formula E is an alkenyl group

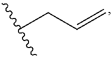 [SMILES: *CC=C]

there can be a double bond migration impurity of formula

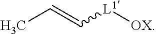

Accordingly, in certain embodiments, when G is an alkenyl group

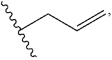 [SMILES: *CC=C]

a compound of formula E comprises an impurity of formula

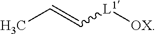

At step S-3, G of a compound of formula E, which is a carboxylic acid having a suitable protecting group or a functional group that can be reacted to form a carboxylic acid, is converted into the carboxylic acid of a fragment compound of formula F-3. In certain embodiments, G is an alkenyl group, and the compound of formula E is oxidized to form the fragment compound of formula F-3. The oxidation of the compound of formula E can be performed using known oxidation cleavage conditions, such as by using potassium permanganate, ozone/hydrogen peroxide, or ruthenium (III) chloride/sodium periodate. In certain embodiments, the oxidation of the compound of formula E is performed using ruthenium (III) chloride/sodium periodate.

In some embodiments, a compound of formula E wherein G is

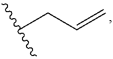 [SMILES: *CC=C]

said compound is oxidized to form compound formula

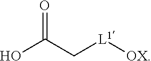

In some embodiments, a compound of formula E wherein G is an alkenyl group

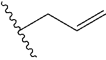 [SMILES: *CC=C]

comprises an impurity of formula

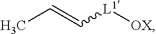

said compound is oxidized to form an impurity of formula

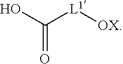

Thus, in some embodiments, the compounds of the present invention prepared using a compound of formula F-3 may include or may be prepared from mixtures of oxidative cleavage products.

According to one embodiment, a fragment compound of formula F-3-a is generally prepared according to Scheme F set forth below:

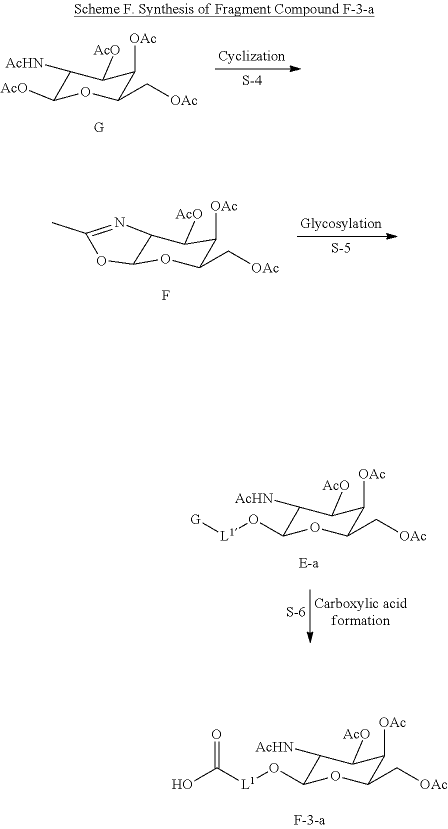

In Scheme F above, each of L¹, L^{1′}, and G is as defined and in classes and subclasses as described herein.

At step S-4, a compound of formula G is treated with a suitable Lewis acid to afford a compound of formula F by an intramolecular cyclization reaction. Suitable Lewis acids include those that are well known in the art, such as boron trifluoride etherates, thioetherates, and alcohol complexes, dicyclohexylboron triflate, trimethylsilyl triflate, tetrafluoroboric acid, aluminum isoproxide, silver triflate, silver tetrafluoroborate, titanium trichloride, tin tetrachloride, scandium triflate, copper (II) triflate, zinc iodide, zinc bromide, zinc chloride, ferric bromide, and ferric chloride, or a montmorillonite clay. Suitable Lewis acids may also include Brønsted acids, such as hydrochloric acid, toluenesulfonic acid, trifluoroacetic acid, or acetic acid. In certain embodiments, a compound of formula G is treated with trimethylsilyl triflate to afford a compound of formula F.

At step S-5, glycosylation of the compound of formula F affords a compound of formula E-a. In certain embodiments, this glycosylation is performed by treating the compound of formula F with alcohol compound of formula

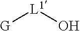

to afford the glycosylation product compound E-a, wherein G is a carboxylic acid having a suitable carboxylate protecting group or a functional group that can be reacted to form a carboxylic acid.

In some embodiments, when G of an alcohol compound of formula

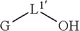

is an alkenyl group

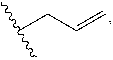 [SMILES: *CC=C]

there can be a double bond migration impurity of formula

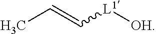

Accordingly, in certain embodiments, when G is an alkenyl group

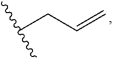 [SMILES: *CC=C]

a compound of formula E-a comprises an impurity of formula

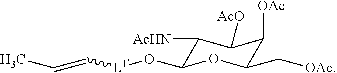

At step S-6, G of a compound of formula E-a, which is a carboxylic acid having a suitable protecting group or a functional group that can be reacted to form a carboxylic acid, is converted into the carboxylic acid of a fragment compound of formula F-3-a. In certain embodiments, G is an alkenyl group, and the compound of formula E-a is oxidized to form the fragment compound of formula F-3-a. The oxidation of the compound of formula E-a can be performed using known oxidation cleavage conditions, such as by using potassium permanganate, ozone/hydrogen peroxide, or ruthenium (III) chloride/sodium periodate. In certain embodiments, the oxidation of the compound of formula E-a is performed using ruthenium (III) chloride/sodium periodate.

In some embodiments, a compound of formula E-a wherein G is

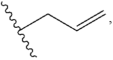 [SMILES: *CC=C]

said compound is oxidized to form compound

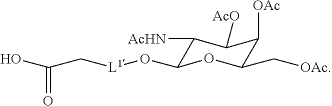

In some embodiments, a compound of formula E-a wherein G is an alkenyl group

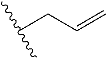 [SMILES: *CC=C]

comprises an impurity of formula

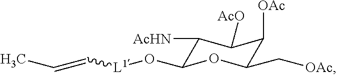

Which is oxidized to form an impurity of formula

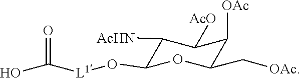

Thus, in some embodiments, the compounds of the present invention may include or may be prepared from mixtures of oxidative cleavage products.

3. Synthesis of a Compound of Formula D-a

According to one embodiment, a Compound of Formula D-a is generally prepared according to Scheme C set forth below:

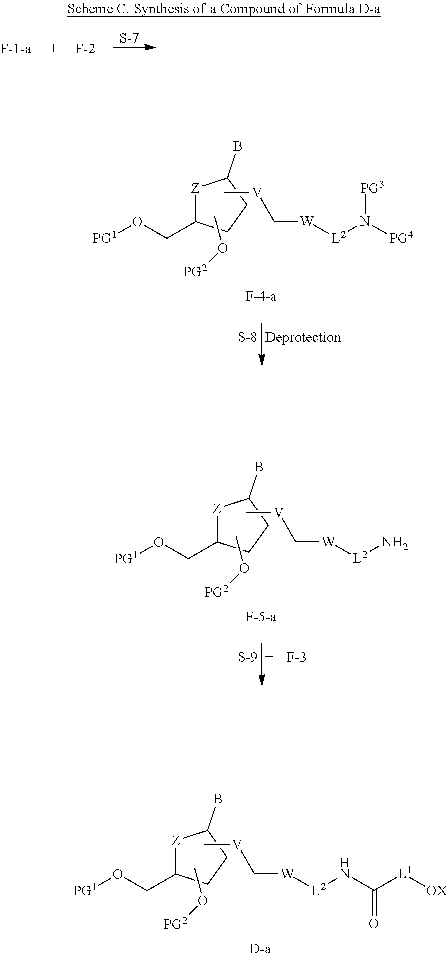

Scheme C above shows a general method for preparing fragment compound of formula D-a or a salt thereof from fragment compounds of formula F-1-a and F-2. In Scheme C above, each of PG¹, PG², PG³, PG⁴, B, L¹, X, L², W, V, and Z is as defined and in classes and subclasses as described herein.

At step S-7, substitution of the thiomethyl group of the fragment compound of formula F-1-a using the fragment compound of formula F-2 affords a fragment compound of formula F-4-a. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

The PG³ and PG⁴ groups of the fragment compounds of formula F-2 and F-4-a are each independently hydrogen or a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of PG³ and PG⁴ groups of the fragment compounds of formula F-2 and F-4-a include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. In certain embodiments, PG³ and PG⁴ groups of the fragment compounds of formula F-2 and F-4-a do not include trifluoroacetyl.

In other embodiments, the PG³ and PG⁴ groups of the fragment compounds of formula F-2 and F-4-a are taken together with their intervening nitrogen atom to form a heterocyclic protecting group, such as phthalimide, pyrrole or pyrrolidine-2,5-dione. In certain embodiments, PG³ and PG⁴ groups of the fragment compounds of formula F-2 and F-4-a are not taken together with their intervening nitrogen to form phthalimide.

In certain embodiments, the PG³ group of the fragment compounds of formula F-2 and F-4-a is Fmoc and the PG⁴ group of the fragment compounds of formula F-2 and F-4-a is hydrogen, or vice versa.

At S-8, removal of protecting groups (e.g., both PG³ and PG⁴ or either of PG³ or PG⁴ independently) of the fragment compound of formula F-4-a affords a fragment compound of formula F-5-a or a salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) of the fragment compound of formula F-4-a are removed by acid hydrolysis. It will be appreciated that upon acid hydrolysis of the protecting groups of the fragment compound of formula F-4-a, a salt compound of the fragment compound of formula F-5-a thereof is formed. For example, when an acid-labile protecting group of the fragment compound of formula F-4-a is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a compound of formula F-5-a are contemplated.

In other embodiments, the protecting groups (e.g., both PG³ and PG⁴ or either of PG³ or PG⁴ independently) of formula F-4-a are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

At step S-9, the fragment compounds of formula F-3 and F-5-a are coupled under suitable amide forming conditions to afford the compound of formula D-a, wherein W is —O—, —S—, or —NR—, and R is as described herein. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with the amine of the fragment compound of formula F-5-a, wherein W is —O—, —S—, or —NR—, and R is as described herein. In certain embodiments, the carboxylic acid of the fragment compound of formula F-3 is converted to an activated ester by reacting with a mixture of NHS (N-hydroxysuccinimide and EDC [1-ethyl-3-(3-dimethylaminopropyl)carbodiimide].

According to one embodiment, a Compound of Formula D-a is generally prepared according to Scheme D set forth below:

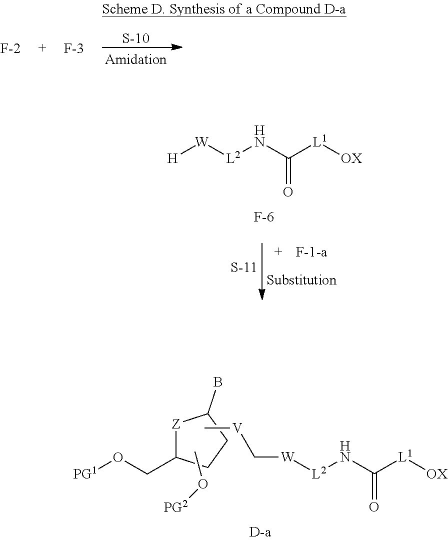

Scheme D above shows a general method for preparing a compound of formula D-a from the fragment compounds of formula F-2 and F-3. In Scheme D above, each of PG¹, PG², PG³, PG⁴, B, L¹, L², V, W, X, and Z is as defined and in classes and subclasses as described herein.

At step S-10, the fragment compounds of formula F-2 and F-3 are coupled under suitable amide forming conditions to afford the fragment compound of formula F-6, wherein W is —O—, —S—, or —NR—, and R is as described herein. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the protecting groups PG³ and PG⁴ on the fragment compound of formula F-2 is removed before reacting with the fragment compound of formula F-3. In certain embodiments, the carboxylic acid of the fragment compound of formula F-3 is converted to an activated ester, followed by reacting with the amine of the fragment compound of formula F-2, wherein W is —O—, —S—, or —NR—, and R is as described herein. In certain embodiments, the carboxylic acid of the fragment compound of formula F-3 is converted to an activated ester by reacting with a mixture of NHS (N-hydroxysuccinimide and EDC [1-ethyl-3-(3-dimethylaminopropyl)carbodiimide].

At step S-11, substitution between a compound of formula F-6 and a compound of formula F-1-a occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

4. Synthesis of a Compound of Formula A-a or A1-a

According to one embodiment, a compound of formula A-a or A1-a is generally prepared according to Scheme E set forth below:

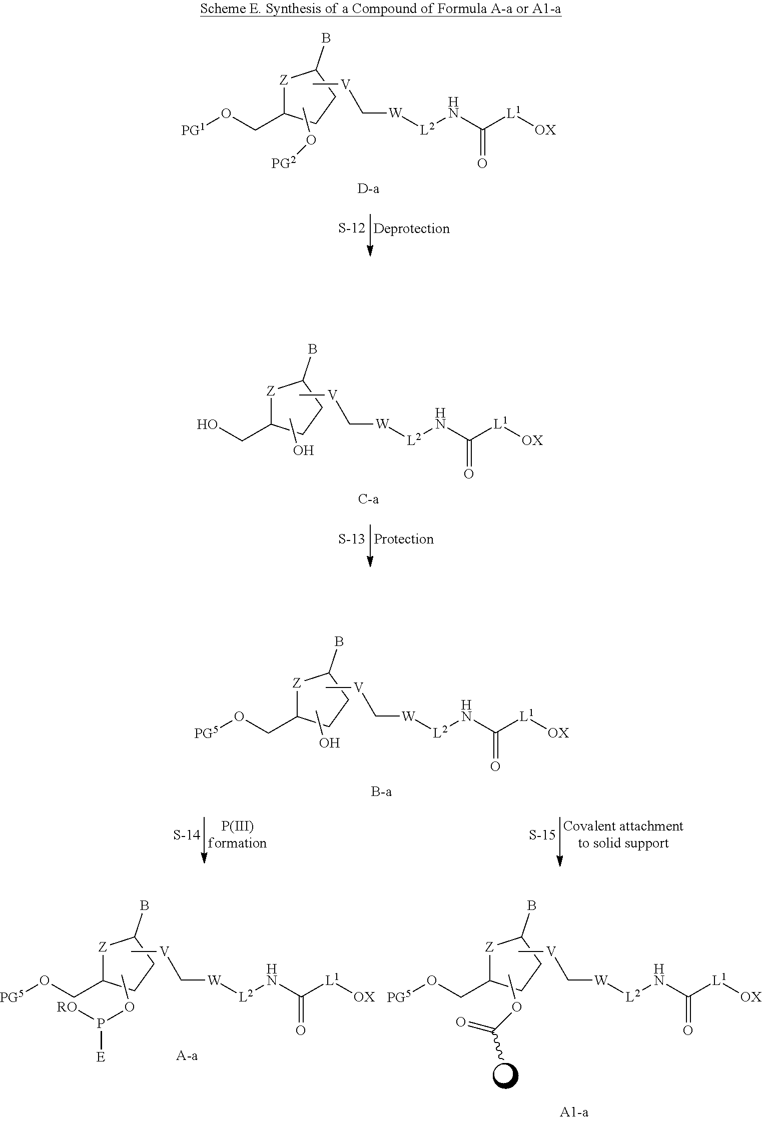

In Scheme E above, each of PG¹, PG², PG⁵, B, E, L¹, L², R, V, W, X, and Z is as defined and in classes and subclasses as described herein.

At step S-12, removal of both protecting groups PG¹ and PG² of the compound of formula affords a compound of formula C-a. In certain embodiments, PG¹ and PG² comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

At step S-13, the 5′-hydroxyl group of a compound of formula C-a is selectively protected to afford a compound of formula B-a. In certain embodiments, the protecting group PG⁵ used for the selective protection of the 5′-hydroxyl group of a compound of formula C-a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In certain embodiments, each of the aforementioned synthetic steps may be performed sequentially with isolation of each intermediate D-a, C-a, and B-a performed after each step. Alternatively, each of steps S-9, S-11, S-12, and S-13, as depicted in Scheme C, D and E above, may be performed in a manner whereby no isolation of any one of intermediates D-a, C-a, and B-a is performed.

At step S-14, a compound of formula B-a is treated with a P(III) forming reagent to afford a compound of formula A-a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite.

In certain embodiments, a compound of formula B-a comprises a hydroxyl group at the 3′ position

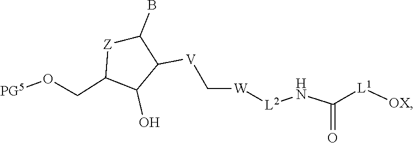

and a compound of formula A-a comprises a phosphoramidite group at the 3′ position:

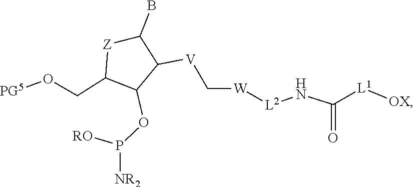

wherein:
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

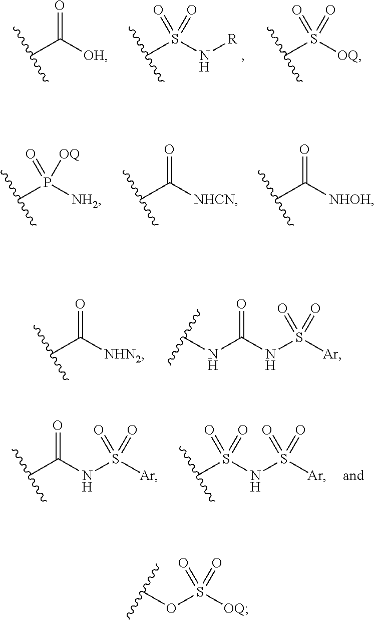

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

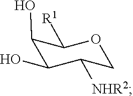

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

At step S-15, in an alternative embodiment, a compound of formula B-a is covalently attached to a solid support to afford a compound of formula A1-a. In certain embodiments, a compound of formula B-a is covalently attached to a solid support through a succinic acid linking group. In certain embodiments, a compound of formula B-a comprises a hydroxyl group at the 3′ position:

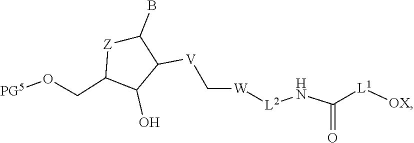

and a compound of formula A1-a comprises a solid support at the 3′ end:

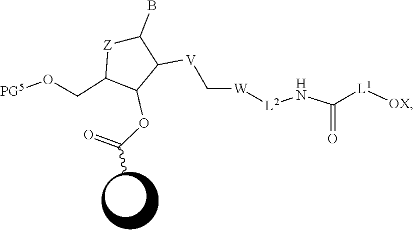

wherein each of PG, B, L¹, L², V, W, X, and Z is as defined and in classes and subclasses as described herein.

According to one alternative embodiment, a compound of formula A1-a is generally prepared according to Scheme F set forth below:

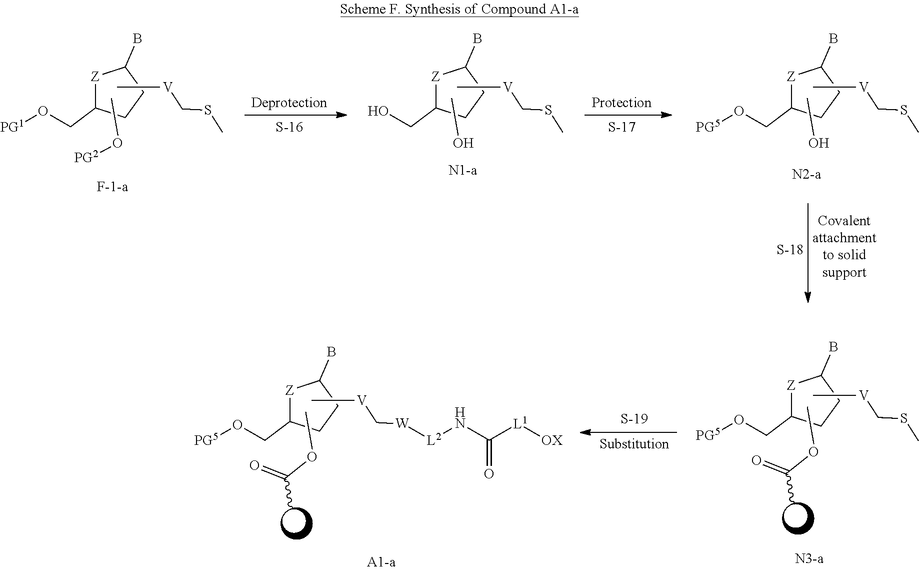

At step S-16, removal of both protecting groups PG¹ and PG² of the compound of formula affords a compound of formula N1-a. In certain embodiments, PG¹ and PG² comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

At step S-17, the 5′-hydroxyl group of a compound of formula N1-a is selectively protected to afford a compound of formula N2-a. In certain embodiments, the protecting group PG⁵ used for the selective protection of the 5′-hydroxyl group of a compound of formula N1-a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

At step S-18, in an alternative embodiment, a compound of formula N2-a is covalently attached to a solid support to afford a compound of formula N3-a. In certain embodiments, a compound of formula N2-a is covalently attached to a solid support through a succinic acid linking group.

At step S-19, the substitution reaction between a compound of formula N3-a with a compound of formula F-6 to afford a compound of formula A1-a occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

According to one alternative embodiment, a compound of formula A1-a is generally prepared according to Scheme G set forth below:

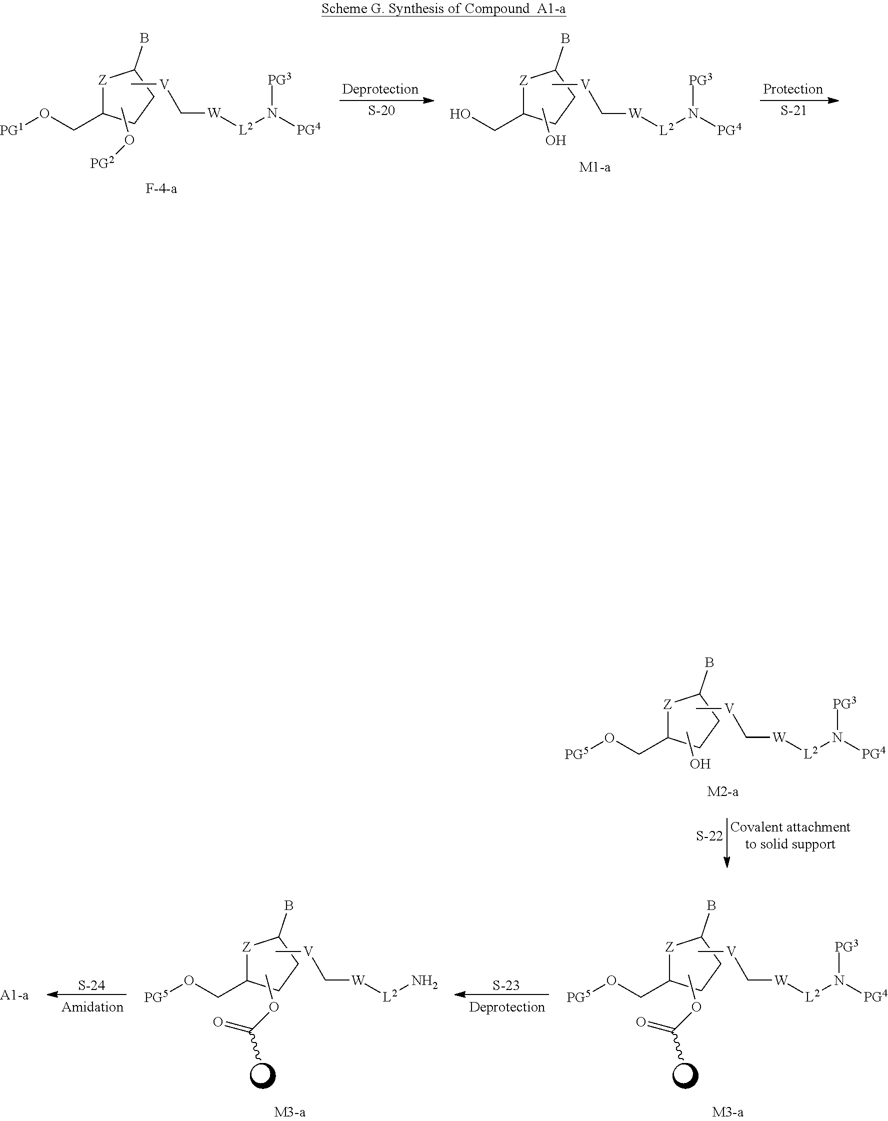

At step S-20, removal of both protecting groups PG¹ and PG² of the fragment compound of formula F-4-a affords a compound of formula M1-a. In certain embodiments, PG¹ and PG² comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

At step S-21, the 5′-hydroxyl group of a compound of formula M1-a is selectively protected to afford a compound of formula M2-a. In certain embodiments, the protecting group PG⁵ used for the selective protection of the 5′-hydroxyl group of a compound of formula M1-a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

At step S-22, in an alternative embodiment, a compound of formula M2-a is covalently attached to a solid support to afford a compound of formula M3-a. In certain embodiments, a compound of formula M2-a is covalently attached to a solid support through a succinic acid linking group.

At step S-23, removal of protecting groups (e.g., both PG³ and PG⁴ or either of PG³ or PG⁴ independently) of the compound of formula M3-a affords a compound of formula M4-a or a salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) of the compound of formula M3-a are removed by acid hydrolysis. It will be appreciated that upon acid hydrolysis of the protecting groups of the compound of formula M3-a, a salt compound of the compound of formula M4-a thereof is formed. For example, where an acid-labile protecting group of the compound of formula M3-a is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a compound of formula M4-a are contemplated.

In other embodiments, the protecting groups (e.g., both PG³ and PG⁴ or either of PG³ or PG⁴ independently) of formula M3-a are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

At step S-24, the compounds of formula M4-a and the fragment compound of formula F-3 are coupled under suitable amide forming conditions to afford the compound of formula A1-a, wherein W is —O—, —S—, or —NR—, and R is as described herein. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with the amine of the compound of formula M4-a, wherein W is —O—, —S—, or —NR—, and R is as described herein. In certain embodiments, the carboxylic acid of the fragment compound of formula F-3 is converted to an activated ester by reacting with a mixture of NHS (N-hydroxysuccinimide and EDC [1-ethyl-3-(3-dimethylaminopropyl)carbodiimide].

According to one alternative embodiment, a fragment compound of formula B-a is generally prepared according to Scheme H set forth below:

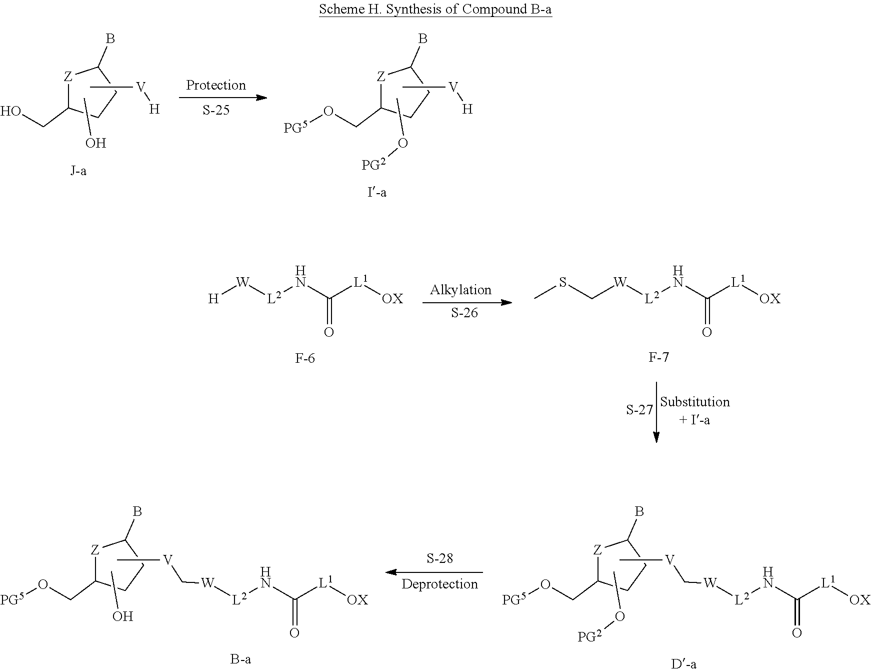

At step S-25, a compound of formula J-a is protected to afford a compound of formula I′-a. In certain embodiments, the protecting groups PG⁵ and PG² used for the protection of the hydroxyl groups of a compound of formula J-a include suitable hydroxyl protecting groups.

Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

In certain embodiments, the protecting group PG⁵ used for protection of the 5′-hydroxyl group of a compound of formula I′-a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

At step S-26, a fragment compound of formula F-6 is alkylated with a mixture of DMSO and acetic anhydride under acidic conditions. In certain embodiments, when —W—H is a hydroxyl group, the mixture of DMSO and acetic anhydride in the presence of acetic acid forms (methylthio)methyl acetate in situ via the Pummerer rearrangement which then reacts with the hydroxyl group of the fragment compound of formula F-6 to provide a monothioacetal functionalized fragment compound of formula F-7.

At step S-27, the substitution reaction between a fragment compound of formula F-7 with a compound of formula I′-a to afford a compound of formula D′-a occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

At step S-28, the selective removal of protecting group PG² of the compound of formula D′-a affords a compound of formula B-a. In certain embodiments, PG² is a suitable hydroxyl protecting groups that can be selective removed in the presence of a second hydroxyl group. Suitable hydroxyl protecting groups that can be chosen for this purpose are described in detail in Protecting Groups in Organic Synthesis, T. W. Greene and P. G. M. Wuts, 3P edition, John Wiley & Sons, 1999, the entirety of each of which is herein incorporated by reference.

5. Synthesis of a Nucleic Acid or Analogue Thereof Compound P4-a

According to one alternative embodiment, a nucleic acid or analogue thereof compound P4-a is generally prepared according to Scheme I set forth below:

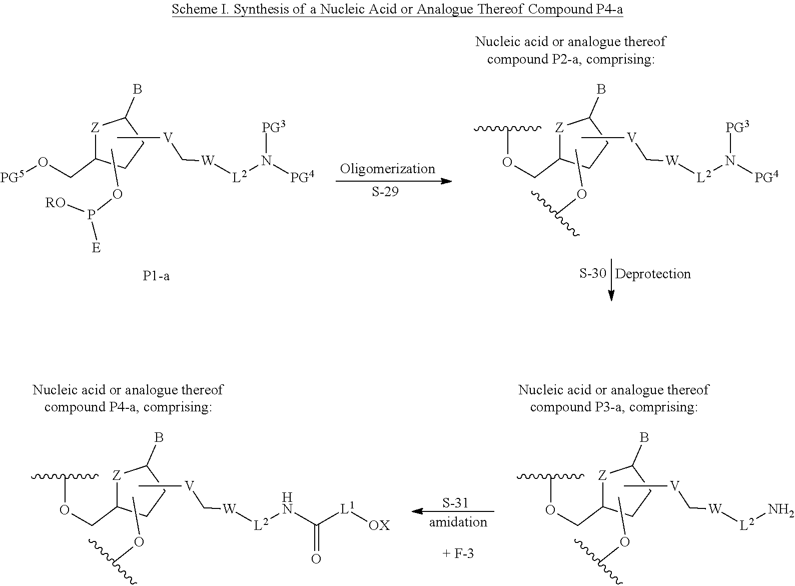

At step S-29, a compound formula P1-a is subjected to nucleic acid or analogue thereof forming conditions preformed using known and commonly applied processes to prepare nucleic acids or analogues thereof in the art. For example, the compound of formula P1-a is coupled to a solid supported nucleic acid or analogue thereof bearing a 5′-hydoxyl group. Further steps can comprise one or more deprotections, couplings, phosphite oxidation, and/or cleavage from the solid support to provide nucleic acids or analogues thereof of various nucleotide lengths, including the nucleic acid or analogue thereof compound P2-a.

At step S-30, removal of protecting groups (e.g., both PG³ and PG⁴ or either of PG³ or PG⁴ independently) of the nucleic acid or analogue thereof compound P2-a affords a nucleic acid or analogue thereof compound P3-a or a salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) of the nucleic acid or analogue thereof compound P2-a are removed by acid hydrolysis. It will be appreciated that upon acid hydrolysis of the protecting groups of nucleic acid or analogue thereof compound P2-a, a salt compound of the nucleic acid or analogue thereof compound P3-a thereof may be formed. For example, where an acid-labile protecting group of the nucleic acid or analogue thereof compound P2-a is removed by treatment with an acid such as hydrochloric acid, then the resulting amine compound may be formed as its hydrochloride salt. One of ordinary skill in the art would recognize that a wide variety of acids are useful for removing amino protecting groups that are acid-labile and therefore a wide variety of salt forms of the nucleic acid or analogue thereof compound P3-a are contemplated.

In other embodiments, the protecting groups (e.g., both PG³ and PG⁴ or either of PG³ or PG⁴ independently) of nucleic acid or analogue thereof compound P2-a are removed by base hydrolysis. In some embodiments, the protecting groups PG³ or PG⁴ of the nucleic acid or analogue thereof compound P2-a is a Fmoc or trifluoroacetyl protecting group that can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

At step S-31, the nucleic acid or analogue thereof compound P3-a and the fragment compound of formula F-3 are coupled under suitable amide forming conditions to afford the nucleic acid or analogue thereof compound P4-a, wherein W is —O—, —S—, or —NR—, and R is as described herein. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with the amine of the nucleic acid or analogue thereof compound P3-a, wherein W is —O—, —S—, or —NR—, and R is as described herein. In certain embodiments, the carboxylic acid of the fragment compound of formula F-3 is converted to an activated ester by reacting with a mixture of NHS (N-hydroxysuccinimide and EDC [1-ethyl-3-(3-dimethylaminopropyl)carbodiimide].

As defined generally above, B is a nucleobase or hydrogen. As used herein, “nucleobase” refers to a heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). Accordingly, the present invention provides a method for preparing a compound of formula A where the nucleobase is generally either a purine or pyrimidine base. In some embodiments, the nucleobase can also include the common bases guanine (G), cytosine (C), adenine (A), thymine (T), or uracil (U), or derivatives thereof, such as protected derivatives suitable for use in the preparation of oligionucleotides. In some embodiments, each of nucleobases G, A, and C independently comprises a protecting group selected from isobutyryl, phenoxyacetyl, isopropylphenoxyacetyl, benzoyl, and acetyl. Nucleobase analogues can duplex with other bases or base analogues in dsRNAs. Nucleobase analogues include those useful in the compounds and methods of the invention, e.g., those disclosed in U.S. Pat. Nos. 5,432,272 and 6,001,983 to Benner and U.S. Patent Publication No. 20080213891 to Manoharan, which are herein incorporated by reference. Non-limiting examples of nucleobases include hypoxanthine (I), xanthine (X), 30-D-ribofuranosyl-(2,6-diaminopyrimidine) (K), 3-O-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivatives thereof (Schweitzer et al., J. Org. Chem., 59:7238-7242 (1994); Berger et al., Nucleic Acids Research, 28(15):2911-2914 (2000); Moran et al., J. Am. Chem. Soc., 119:2056-2057 (1997); Morales et al., J. Am. Chem. Soc., 121:2323-2324 (1999); Guckian et al., J. Am. Chem. Soc., 118:8182-8183 (1996); Morales et al., J. Am. Chem. Soc., 122(6):1001-1007 (2000); McMinn et al., J. Am. Chem. Soc., 121:11585-11586 (1999); Guckian et al., J. Org. Chem., 63:9652-9656 (1998); Moran et al., Proc. Natl. Acad. Sci., 94:10506-10511 (1997); Das et al., J. Chem. Soc., Perkin Trans., 1:197-206 (2002); Shibata et al., J. Chem. Soc., Perkin Trans., 1: 1605-1611 (2001); Wu et al., J. Am. Chem. Soc., 122(32):7621-7632 (2000); O'Neill et al., J. Org. Chem., 67:5869-5875 (2002); Chaudhuri et al., J. Am. Chem. Soc., 117:10434-10442 (1995); and U.S. Pat. No. 6,218,108.). Base analogues may also be a universal base.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases do not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-O-D-ribo furanosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. Nucleic Acids Res. 1995 Nov. 11; 23(21):4363-70; Loakes et al., 3-Nitropyrrole and 5-nitroindole as universal bases in primers for DNA sequencing and PCR. Nucleic Acids Res. 1995 Jul. 11; 23(13):2361-6; Loakes and Brown, 5-Nitroindole as a universal base analogue. Nucleic Acids Res. 1994 Oct. 11; 22(20):4039-43).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, bifumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

6. Methods of the Invention

According to one aspect, the present invention provides a method for preparing a compound of formula A:

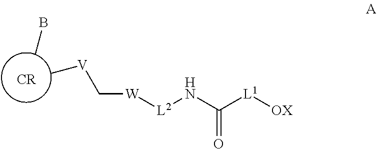

or a salt thereof, wherein:

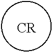

is

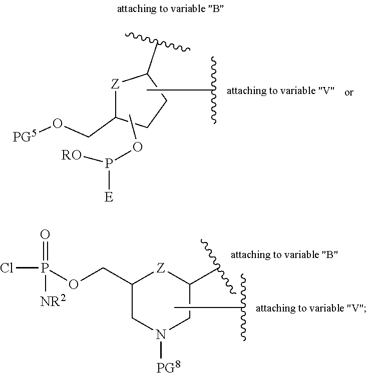

PG⁵ is hydrogen or a suitable hydroxyl protecting group;
PG⁸ is hydrogen or a suitable nitrogen protecting group;
B is a nucleobase or hydrogen;
E is halogen or NR₂;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

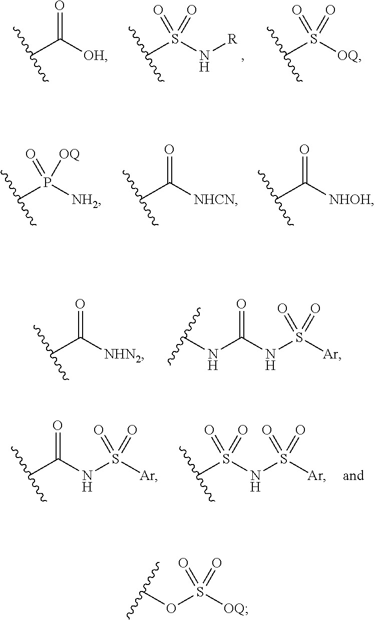

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

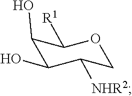

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—, comprising the steps of:
(a) providing a compound of formula B:

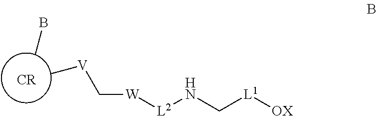

or a salt thereof, wherein

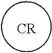

is

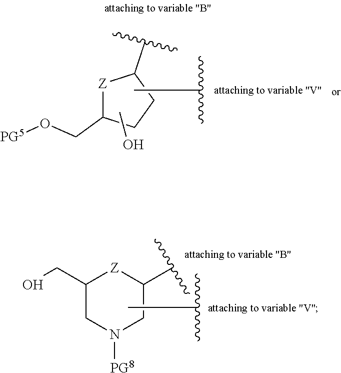

and
(b) reacting said compound of formula B with a P(III) or P(V) forming reagent to form a compound of formula A.

According to one aspect, the present invention provides a method for preparing a compound of formula A-a:

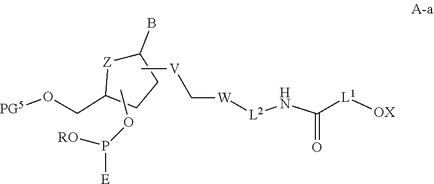

or a salt thereof, wherein:
PG⁵ is hydrogen or a suitable hydroxyl protecting group;
B is a nucleobase or hydrogen;
E is halogen or NR₂;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

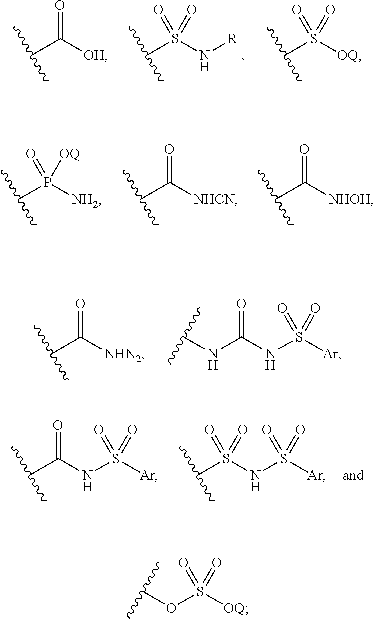

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

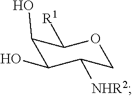

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula B-a:

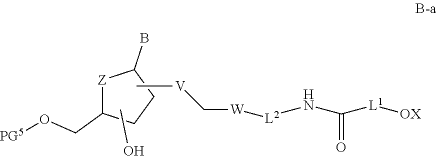

or a salt thereof, and
(b) reacting said compound of formula B-a with a P(III) forming reagent to form a compound of formula A-a.

According to one embodiment, step (b) above is preformed using 2-cyanoethyl N,N-diisopropylchlorophosphoramidite as a P(III) forming reagent. According to another embodiment, step (b) above is preformed using 2-cyanoethyl phosphorodichloridite as a P(III) forming reagent. One of ordinary skill would recognize that the displacement of a leaving group in a P(III) forming reagent by the hydroxyl moiety of a compound of formula B is achieved either with or without the presence of a suitable base. Such suitable bases are well known in the art and include organic and inorganic bases. In certain embodiments, the base is a tertiary amine such as triethylamine or diisopropylethylamine. In other embodiments, step (b) above is preformed using N,N-dimethylphosphoramic dichloride as a P(V) forming reagent.

In certain aspects, the present invention provides a method for preparing a compound of formula A-a where X is GalNAc and the connectivity and stereochemistry is as shown in the compound of formula A-b:

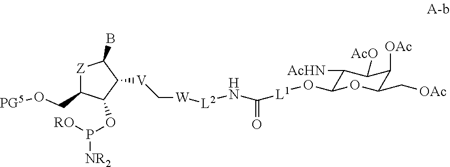

or a salt thereof, wherein each of PG⁵, B, L¹, L², R, V, W, and Z is as defined and in classes and subclasses as described herein,
   comprising the steps of:
(a) providing a compound of formula B-b:

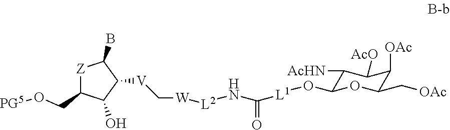

or a salt thereof, and
(b) reacting said compound of formula B-b with a phosphoramidite forming reagent to form a compound of formula A-b.

According to another aspect, the present invention provides a method for preparing a compound of formula A1:

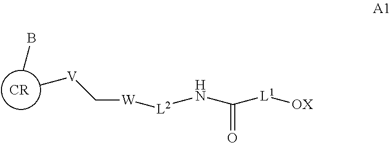

or a salt thereof, wherein:

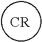

is

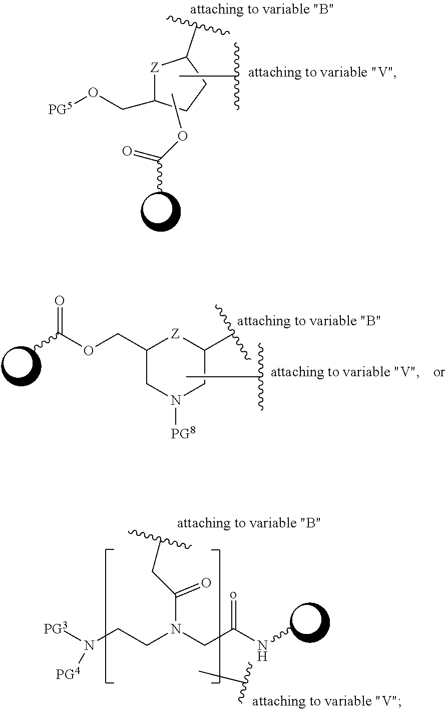

PG³ and PG⁴ are independently hydrogen or a suitable nitrogen protecting group, provided both PG³ and PG⁴ are not hydrogen at the same time;
PG⁵ is hydrogen or a suitable hydroxyl protecting group;
PG⁸ is hydrogen or a suitable nitrogen protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

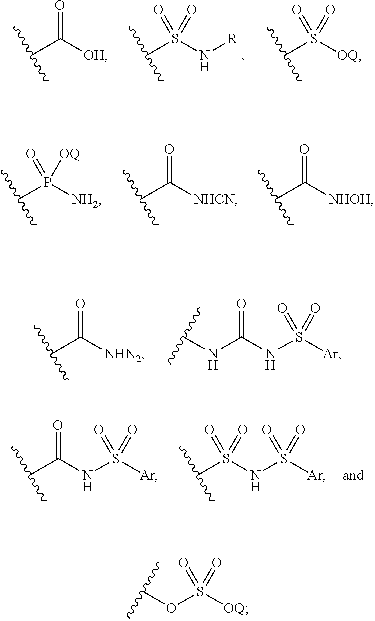

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

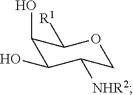

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a solid support of formula

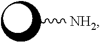

and a compound of formula B:

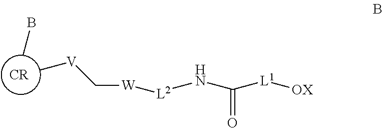

or a salt thereof, wherein

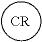

is

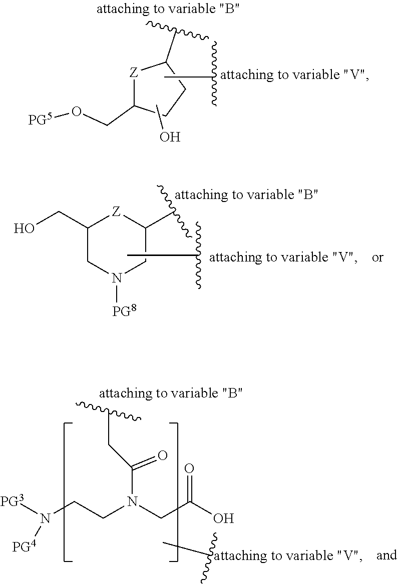

(b) reacting said compound of formula B with the solid support of formula

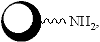

to form a compound of formula A1.

According to another aspect, the present invention provides a method for preparing a compound of formula A1-a:

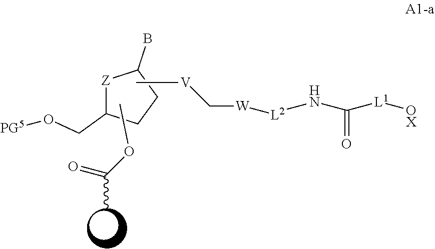

or a salt thereof, wherein:
PG⁵ is a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

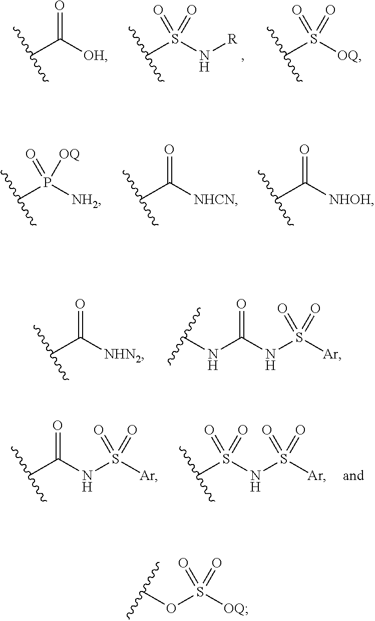

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

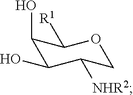

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a solid support of formula

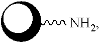

and a compound of formula B-a:

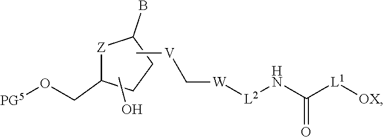

and
(b) reacting said compound of formula B-a with the solid support of formula

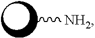

to form a compound of formula A1-a.

In certain embodiments, the hydroxyl group of a compound of formula B-a is covalently attached to a solid support through a succinic acid linking group. One of ordinary skill would recognize that the covalent attachment of a compound of formula B-a to a solid support could be performed by reacting with a dicarboxylic acid compound, or an anhydride thereof, forming an ester with the —OH of the compound of formula B-a and an amide with the —NH₂ of the solid support. Formation of esters appropriate for solid support synthesis are well known in the art, e.g., see, “Advanced Organic Chemistry”, Jerry March, 5^{th} edition, John Wiley and Sons, N.Y.

In certain aspects, the present invention provides a method for preparing a compound of formula A1-a where X is GalNAc and the connectivity and stereochemistry is as shown in the compound of formula A1-b:

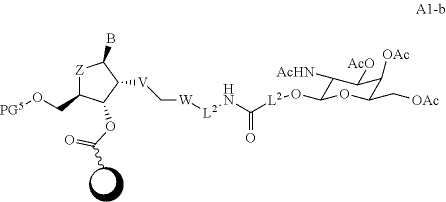

or a salt thereof, wherein each of PG⁵, B, L¹, L², V, W, and Z is as defined and in classes and subclasses as described herein,
   comprising the steps of:
(a) providing a solid support of formula

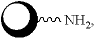

and a compound of formula B-b:

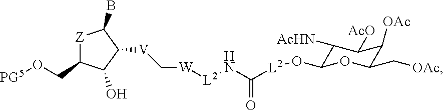

and
(b) reacting said compound of formula B-b with the solid support of formula

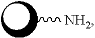

to form a compound of formula A1-b.

According to another aspect, the present invention provides a method for preparing a compound of formula B:

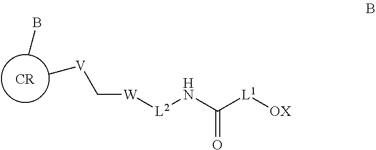

or a salt thereof, wherein:

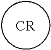

is

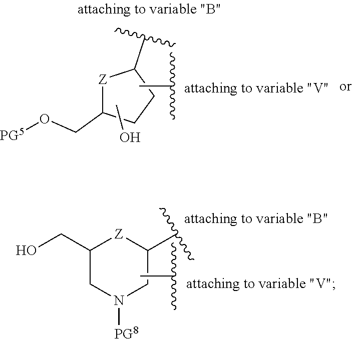

PG⁵ is hydrogen or a suitable hydroxyl protecting group;
PG⁸ is hydrogen or a suitable nitrogen protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

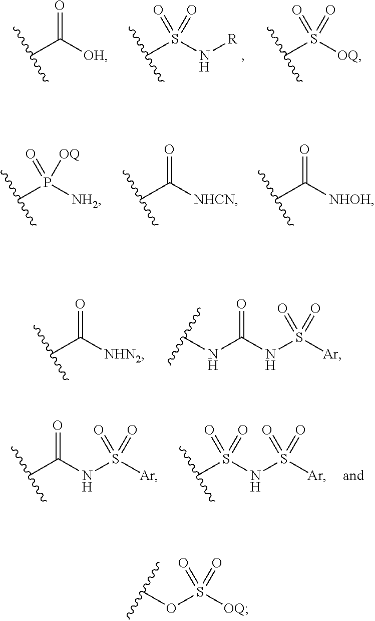

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

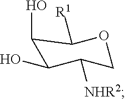

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula C:

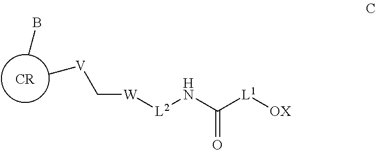

or a salt thereof, wherein

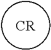

is

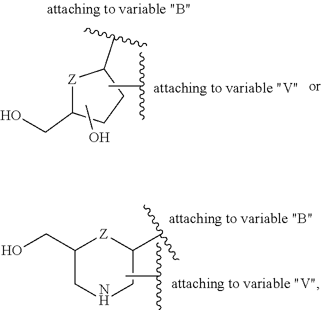

(b) protecting said compound of formula C with a suitable protecting group to form a compound of formula B.

In certain embodiments, the protecting group PG⁸ used for selective protection of a nitrogen group, for example, in formulas A, A1, and B, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another aspect, the present invention provides a method for preparing a compound of formula B-a:

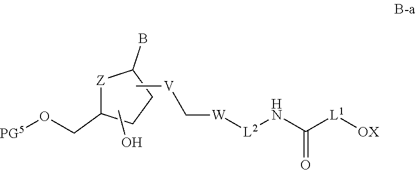

or a salt thereof, wherein:
PG⁵ is a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

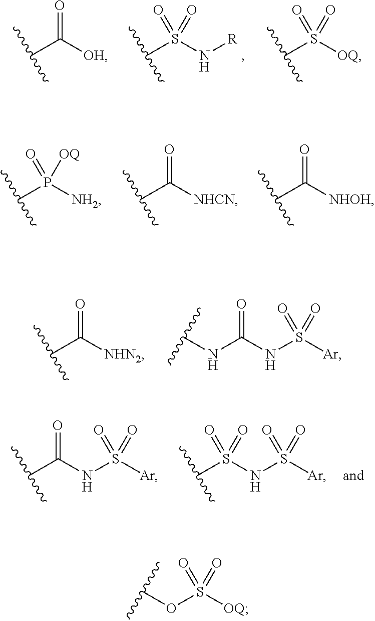

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

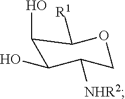

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula C-a:

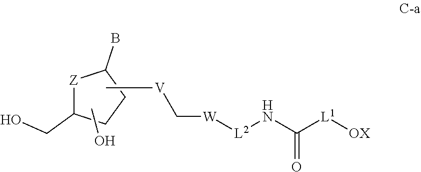

or a salt thereof, and
(b) protecting said compound of formula C-a with a suitable protecting group to form a compound of formula B-a.

According to one embodiment, a compound of formula C or C-a is selectively protected in step (b) above with a suitable protecting group. In some embodiments, the protecting group PG⁵ used for the selective protection of the 5′-hydroxyl group of a compound of formula C includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid. In certain embodiments, PG⁵ is 4,4′-dimethyoxytrityl. One of ordinary skill would recognize that the displacement of a leaving group in a protecting group reagent by the hydroxyl moiety of a compound of formula C or C-a is achieved either with or without the presence of a suitable base. Such suitable bases are well known in the art and include organic and inorganic bases. In certain embodiments, the base is a tertiary amine such as N-methylmorpholine.

In certain aspects, the present invention provides a method for preparing a compound of formula B-a wherein X is GalNAc and the connectivity and stereochemistry is as shown in the compound of formula B-b:

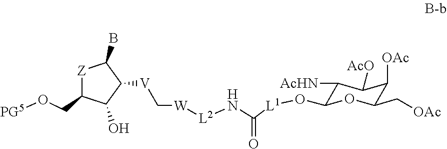

or a salt thereof, wherein each of PG⁵, B, L¹, L², V, W, and Z is as defined and in classes and subclasses as described herein,
   comprising the steps of:
(a) providing a compound of formula C-a:

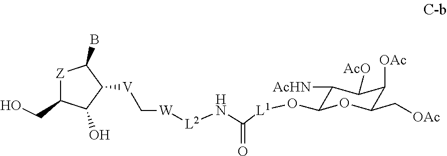

or a pharmaceutically acceptable salt thereof, wherein each of B, L¹, L², V, W, and Z is as defined and in classes and subclasses as described herein, and
(b) protecting said compound of formula C-b with a suitable protecting group to form a compound of formula B-b.

According to another aspect, the present invention provides a method for preparing a compound of formula C:

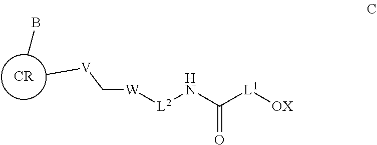

or a salt thereof, wherein

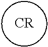

is

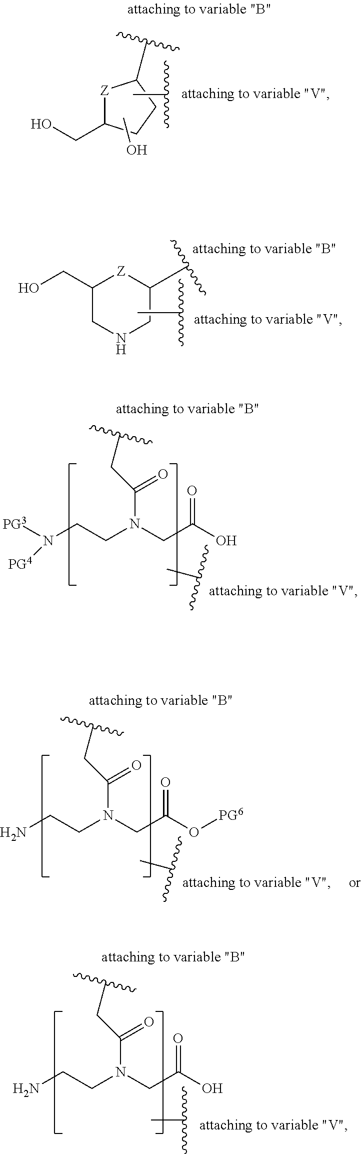

comprising the steps of:
(a) providing a compound of formula D:

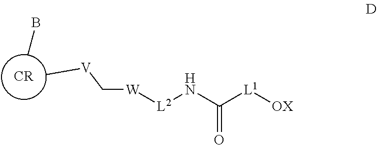

or a salt thereof, wherein

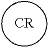

is

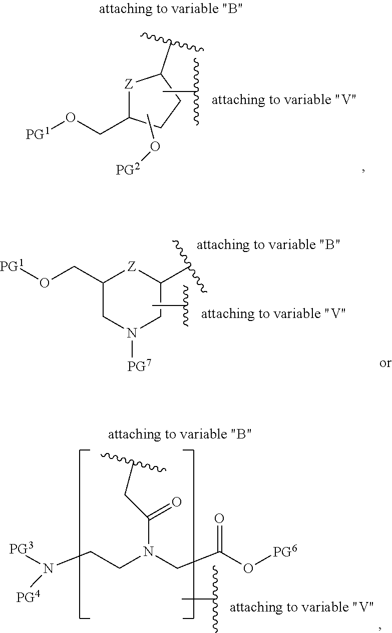

and
(b) deprotecting said compound of formula D to form a compound of formula C, wherein:
PG¹ and PG² are independently hydrogen or a suitable hydroxyl protecting group;
PG³, PG⁴, and PG⁷ are independently hydrogen or a suitable nitrogen protecting group;
PG⁶ is hydrogen or a suitable carboxylate protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

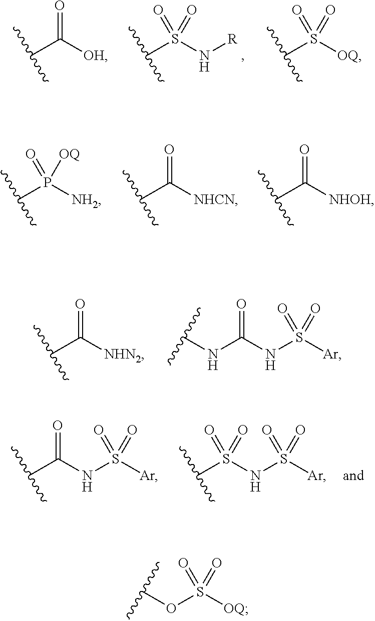

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

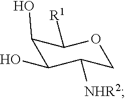

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to another aspect, the present invention provides a method for preparing a compound of formula C-a:

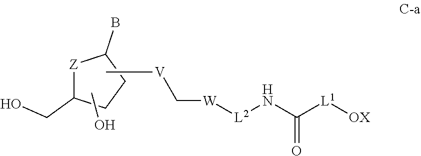

or a salt thereof,
comprising the steps of:
(a) providing a compound of formula D-a:

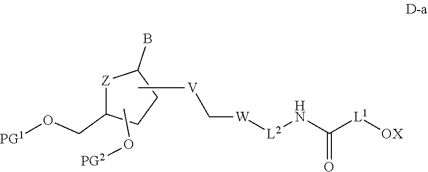

or a salt thereof, and
(b) deprotecting said compound of formula D-a to form a compound of formula C-a, wherein PG¹ and PG² are independently a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

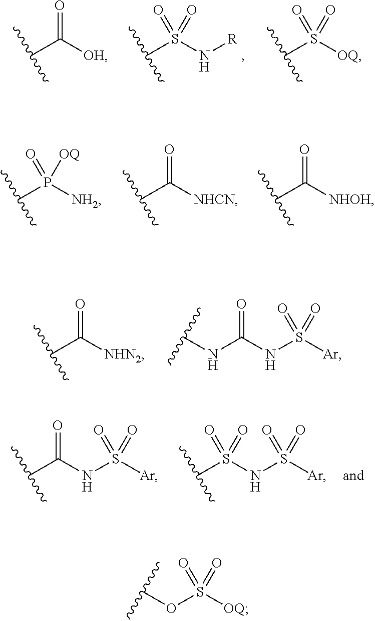

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

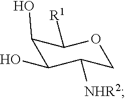

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to one embodiment, PG¹ and PG² removed in step (b) above are selected from suitable hydroxyl protecting groups. Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

In certain embodiments, the PG¹ and PG² groups removed to form a compound of formula C or C-a in step (b) above are taken together to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene. In some embodiments, 1,1,3,3-tetraisopropylidisiloxanylidene is removed under acidic conditions or with fluoride anion. Examples of acids for the removal of silicon-based protecting groups include suitable acids well known in the art such as inorganic acids, e.g., hydrochloric acid, hydrobromic acid, phosphoric acid, nitric acid, sulfuric acid or perchloric acid, or organic acids, e.g., acetic acid, trifluoroacetic acid, p-toluenesulfonic acid, or methanesulfonic acid. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

The PG³, PG⁴, and PG⁷ groups of the compound of formula D or D-a above are a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of PG³ groups of the compound of formula D or D-a include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

In certain embodiments, the protecting group PG⁷ used for selective protection of a nitrogen group, for example, the nitrogen of

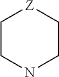

as shown in certain formulas, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In certain aspects, the present invention provides a method for preparing a compound of formula C-a where X is GalNAc and the connectivity and stereochemistry is as shown in the compound of formula C-b:

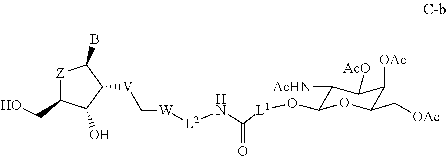

or a salt thereof, wherein each of B, L¹, L², R, V, W, and Z is as defined and in classes and subclasses as described herein,
   comprising the steps of:
   (a) providing a compound of formula D-b:

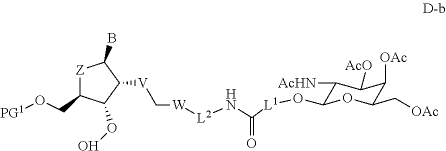

or a salt thereof, and
(b) deprotecting said compound of formula D-b to form a compound of formula C-b.

According to another aspect, the present invention provides a method for preparing a compound of formula D:

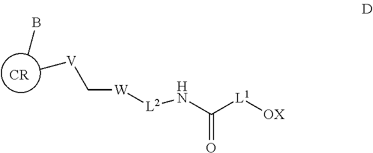

or a salt thereof, wherein:

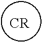

is

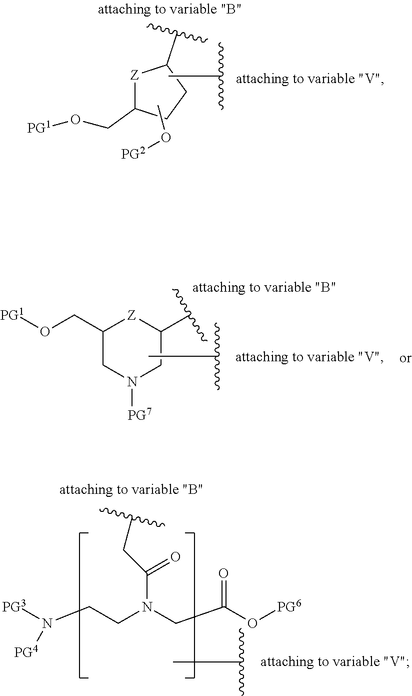

PG¹ and PG² are independently hydrogen or a suitable hydroxyl protecting group;
PG³, PG⁴, and PG⁷ are independently hydrogen or a suitable nitrogen protecting group;
PG⁶ is hydrogen or a suitable carboxylate protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

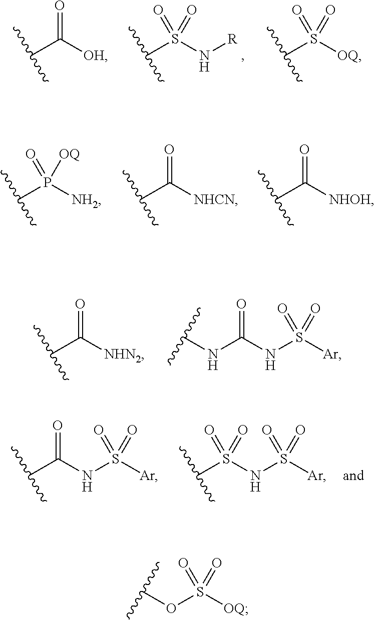

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

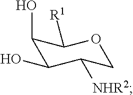

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula F-3:

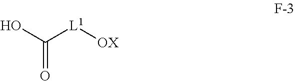

or a salt thereof, and
(b) reacting said fragment compound of formula F-3 with a fragment compound of formula F-5:

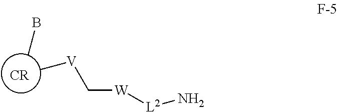

or a salt thereof, to provide the compound of formula D.

According to another aspect, the present invention provides a method for preparing a compound of formula D-a:

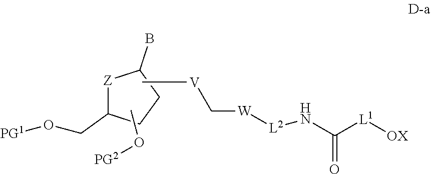

or a salt thereof, wherein:
PG¹ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

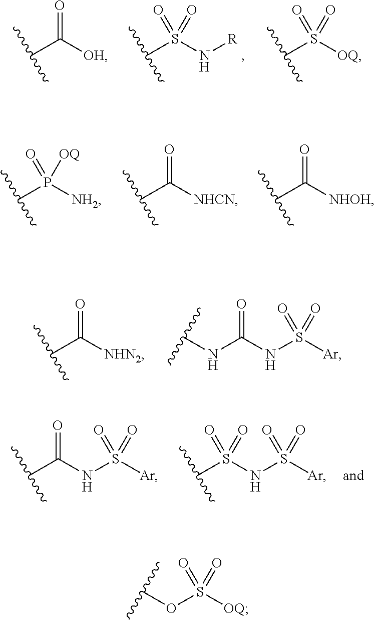

each R is independently selected from hydrogen, alkyl, alkenyl, aromatic, heterocycle, substituted alkyl, and substituted alkenyl; Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

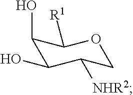

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula F-3:

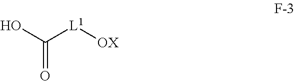

or a salt thereof, and
(b) reacting said fragment compound of formula F-3 with a fragment compound of formula F-5-a:

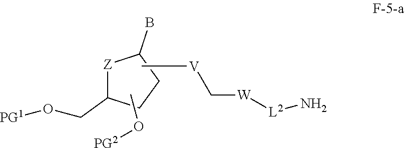

or a salt thereof, to provide the compound of formula D-a.

According to one embodiment, the amidation reaction of step (b) can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with an amine compound. In certain embodiments, the activated ester forming conditions include a mixture of NHS (N-hydroxysuccinimide and EDC [1-ethyl-3-(3-dimethylaminopropyl)carbodiimide].

Without being limited to the current disclosure, the assembly of fragment compound of formula F-3 with the fragment compound of formula F-5 or F-5-a in step (b) could be facilitated using a range of cross-linking technologies. It is within the purview of those having ordinary skill in the art that the carboxylic acid of the fragment compound of formula F-3 and the amine of the fragment compound of formula F-5 or F-5-a could be replaced by suitable coupling moieties that react with each other to covalently link the fragment compound of formula F-3 with the fragment compound of formula F-5 or F-5-a by alternative means. Exemplary cross-linking technologies envisioned for use in the current disclosure also include those listed in Table 1.

TABLE 1
Exemplary Cross-linking Technologies

| Reaction | |
|---|---|
| Type | Reaction Summary |
| Thiol-yne | 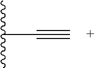 [SMILES: *C#C] |
| | 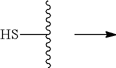 [SMILES: *S] |
| | 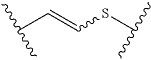 [SMILES: *C=CS*] |
| NHS ester | 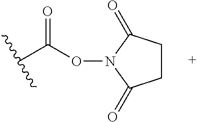 [SMILES: *C(=O)ON1C(=O)CCC1=O] |
| | 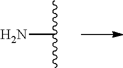 [SMILES: *N] |
| | 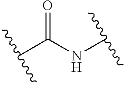 [SMILES: *NC(*)=O] |
| Thiol-ene | 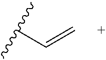 [SMILES: *C=C] |
| | 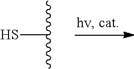 [SMILES: *S] |
| | 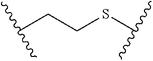 [SMILES: *CCS*] |
| Isocyanate | 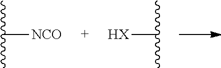 |
| | 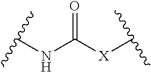 |
| | X = S or NH |
| Epoxide or aziridine | 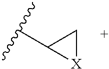 |
| | 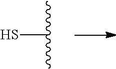 [SMILES: *S] |
| | 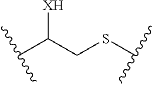 |
| | X = O or NH |
| Aldehyde- aminoxy | 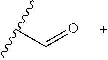 [SMILES: *C=O] |
| | 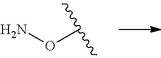 [SMILES: *ON] |
| | 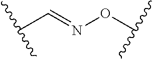 [SMILES: */C=N/O*] |
| Cu-catalyzed- azide-alkyne cycoaddition | 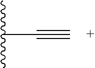 [SMILES: *C#C] |
| | 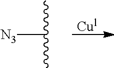 |
| | 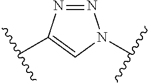 [SMILES: *c1cn(*)nn1] |
| Strain- | Cyclooctyne cycloaddition (with azide, nitrile, or nitrone) |
| promoted cycloaddition | 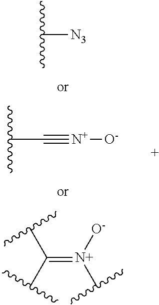 |
| | 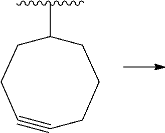 [SMILES: *C1CCC#CCCC1] |
| | 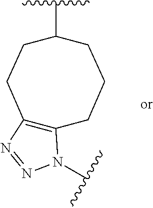 [SMILES: *C1CCCc2c(nnn2*)CC1] |
| | 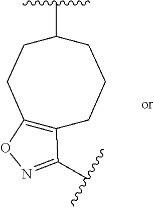 [SMILES: *c1noc2c1CCCC(*)CC2] |
| | 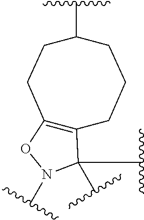 [SMILES: *C1CCCC2=C(CC1)ON(*)C2(*)*] |
| | Norbornene cycloaddition (with azide, nitrile oxide, ot nitrone |
| | 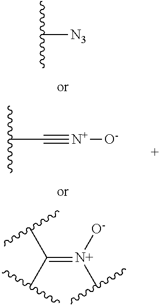 |
| | 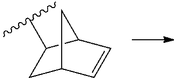 [SMILES: *C1CC2C=CC1C2] |
| | 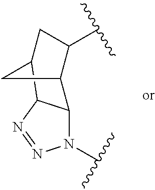 [SMILES: *C1CC2CC1C1C2N=NN1*] |
| | 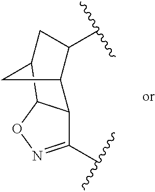 [SMILES: *C1=NOC2C3CC(*)C(C3)C12] |
| | 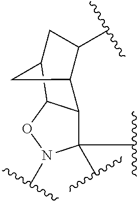 [SMILES: *C1CC2CC1C1C2ON(*)C1(*)*] |
| | Oxanorbornadiene cycloaddition |
| | 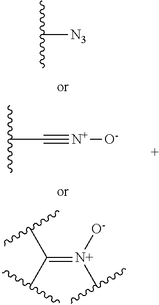 |
| | 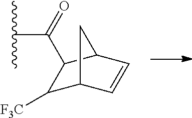 [SMILES: *C(=O)C1C2C=CC(C2)C1C(F)(F)F] |
| | 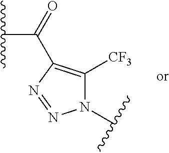 [SMILES: *C(=O)c1nnn(*)c1C(F)(F)F] |
| | 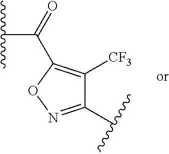 [SMILES: *C(=O)c1onc(*)c1C(F)(F)F] |
| | 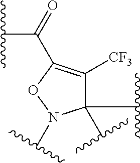 [SMILES: *C(=O)C1=C(C(F)(F)F)C(*)(*)N(*)O1] |
| Staudinger ligation | 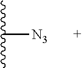 [SMILES: *N=[N+]=[N-]] |
| | 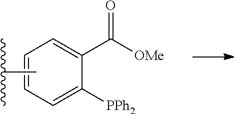 |
| | 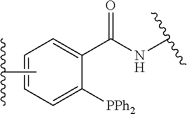 |
| Tetrazine ligation | 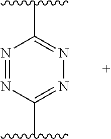 [SMILES: *c1nnc(*)nn1] |
| | 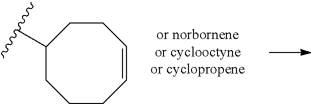 [SMILES: *C1CC/C=C\CCC1] |
| | 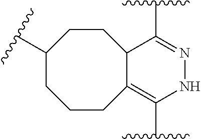 [SMILES: *C1=NNC(*)=C2CCCC(*)CCC12] |
| Photo-induced tetrazole- alkene cycloaddition | 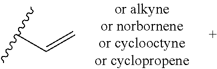 [SMILES: *C=C] |
| | 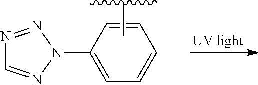 |
| | 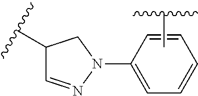 |
| [4 + 1] cycloaddition | 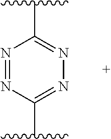 [SMILES: *c1nnc(*)nn1] |
| | 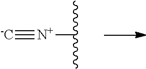 |
| | 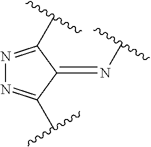 [SMILES: *N=C1C(*)=NN=C1*] |
| Quadricyclane ligation | 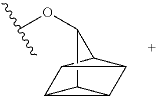 [SMILES: *OC1C2C3C2C2C1C32] |
| | 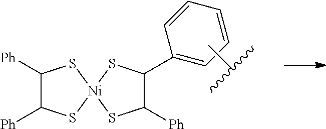 |
| | 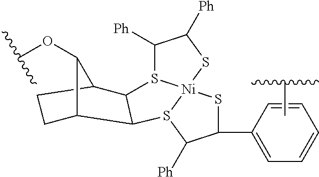 |

Accordingly, in certain embodiments, the present invention provides a compound of formulae

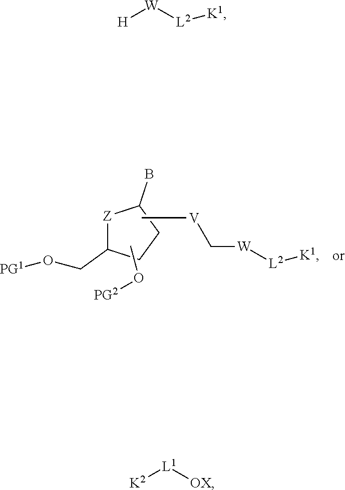

wherein each of PG¹, PG², B, X, L¹, L², V, W, and Z is as defined and in classes and subclasses as described herein, and each of K¹ and K² is independently selected from the coupling moieties listed in Table 1. In some embodiments, the present invention provides a compound of formulae:

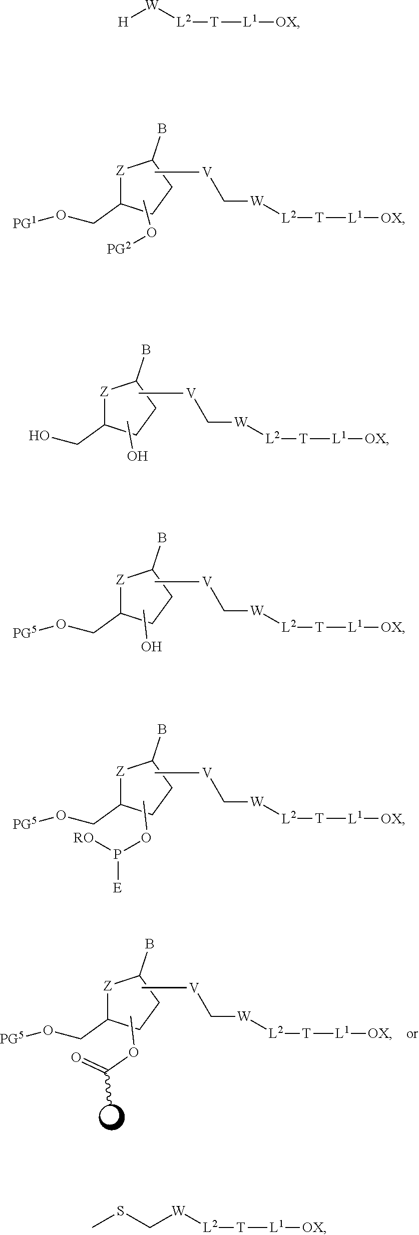

wherein each of PG¹, PG², PG⁵, B, E, X, L¹, L², V, W, and Z is as defined and in classes and subclasses as described herein, and T is selected from the linkers listed in Table 1.

In certain aspects, the present invention provides a method for preparing a compound of formula D-a where X is GalNAc and the connectivity and stereochemistry is as shown in the compound of formula D-b:

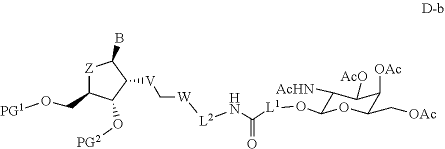

or a pharmaceutically acceptable salt thereof, wherein each of PG¹, PG², B, L¹, L², V, W, and Z is

as defined and in classes and subclasses as described herein, comprising the steps of:

(a) providing a compound of formula F-3-a:

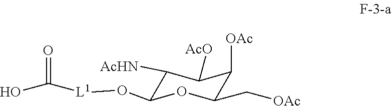

or a salt thereof, and
(b) reacting said compound of formula F-3-a with a compound of formula F-5-b:

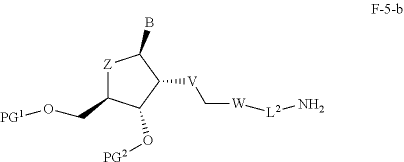

or a salt thereof,
to provide the compound of formula D-b.

According to another aspect, the present invention provides a method for preparing a compound of formula F-3:

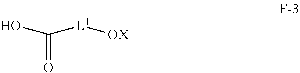

or a salt thereof, comprising the steps of:
(a) providing a compound of formula E:

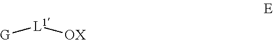

or a salt thereof, and
(b) converting said compound of formula E to a fragment compound of formula F-3, wherein
G is a carboxylic acid having a suitable carboxylate protecting group or a functional group that can be reacted to form a carboxylic acid;
L¹ and L^{1′} each is independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
each Y is independently selected from H, C₁-C₆ alkanyl, C₁-C₆ alkenyl or aryl, including

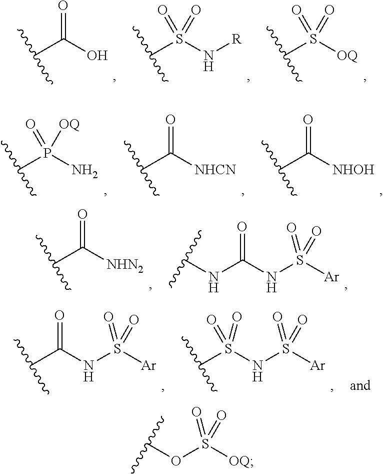

each R is independently selected from hydrogen, alkyl, alkenyl, aromatic, heterocycle, substituted alkyl, and substituted alkenyl; and
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
X is a ligand selected from GalNAc, D-mannose, L-galactose, D-arabinose, L-fucose, polyols, and

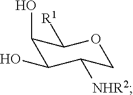

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³); and
R³ is H, C₁-C₆ alkanyl, C₁-C₆ alkenyl, or aryl.

In certain aspects, the present invention provides a method for preparing a fragment compound of formula F-3 where X is GalNAc as shown in the fragment compound of formula F-3-a:

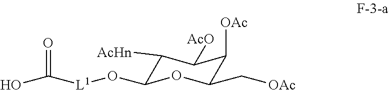

or a salt thereof, comprising the steps of
(a) providing a compound of formula G:

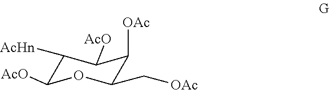

or a salt thereof,
(b) cyclizing said compound of formula G to form a compound of formula F:

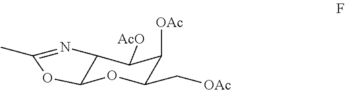 [SMILES: CC(=O)OCC1OC2OC(C)=NC2C(OC(C)=O)C1OC(C)=O]

or a salt thereof,
(c) reacting said compound of formula F with an alcohol compound of formula

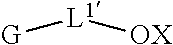

to form a compound of formula E-a:

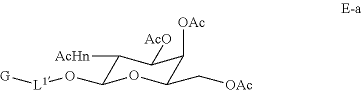

or a salt thereof, and
(d) converting said compound of formula E-a to a compound of formula F-3-a, wherein each of G, L^{1′}, and L¹ is as defined and in classes and subclasses as described herein.

According to one embodiment, step (b) above is performed using a suitable Lewis acid to afford a compound of formula F by an intramolecular cyclization reaction. Suitable Lewis acids include those that are well known in the art, such as boron trifluoride etherates, thioetherates, and alcohol complexes, dicyclohexylboron triflate, trimethylsilyl triflate, tetrafluoroboric acid, aluminum isoproxide, silver triflate, silver tetrafluoroborate, titanium trichloride, tin tetrachloride, scandium triflate, copper (II) triflate, zinc iodide, zinc bromide, zinc chloride, ferric bromide, and ferric chloride, or a montmorillonite clay. Suitable Lewis acids may also include Brønsted acids, such as hydrochloric acid, toluenesulfonic acid, trifluoroacetic acid, or acetic acid. In certain embodiments, a compound of formula G is treated with trimethylsilyl triflate to afford a compound of formula F.

According to another embodiment, reacting said compound of formula F with an alcohol compound at step (c) above comprises a glycosylation. In certain embodiments, the glycosylation is achieved by reacting said compound of formula F with a compound of formula

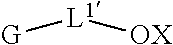

wherein said reaction is performed under suitable glycosylation conditions and wherein:
L^{1′} is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
each Y is independently selected from H, C₁-C₆ alkanyl, C₁-C₆ alkenyl or aryl, including

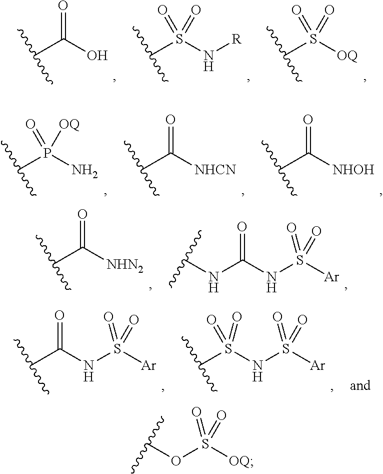

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY where Y is H, C₁-C₆ alkanyl, C₁-C₆ alkenyl or aryl; and
G is a carboxylic acid having a suitable carboxylate protecting group or a functional group that can be reacted to form a carboxylic acid.

Suitable glycosylation conditions can include using any of the Lewis acids mentioned for use in step (b) above. In certain embodiments, the glycosylation of a compound of formula F is performed using trimethylsilyl triflate in a suitable medium. A suitable medium is a solvent or a solvent mixture that, in combination with the combined compounds, may facilitate the progress of the reaction therebetween. The suitable solvent may solubilize one or more of the reaction components, or, alternatively, the suitable solvent may facilitate the agitation of a suspension of one or more of the reaction components. Examples of suitable solvents useful in the present invention are a protic solvent, a halogenated hydrocarbon, an ether, an ester, an aromatic hydrocarbon, a polar or a non-polar aprotic solvent, or any mixtures thereof. Such mixtures include, for example, mixtures of protic and non-protic solvents such as benzene/methanol/water; benzene/water; DME/water, and the like.

These and other such suitable solvents are well known in the art, e.g., see, “Advanced Organic Chemistry”, Jerry March, 5^{th} edition, John Wiley and Sons, N.Y.

According to another embodiment, converting said compound of formula E or E-a to a compound of formula F-3 or F-3-a comprises converting group G of a compound of formula E or E-a to a carboxylic acid containing group. In some embodiments, group G is a carboxylic acid having a suitable protecting group or a functional group that can be reacted to form a carboxylic acid. Suitable carboxylate protecting groups are well known in the art and include those described in detail in Protecting Groups in Organic Synthesis, T. W. Greene and P. G. M. Wuts, 3d edition, John Wiley & Sons, 1999, the entirety of each of which is herein incorporated by reference. Suitable carboxylate protecting groups include, but are not limited to, substituted C₁₋₆ aliphatic esters, optionally substituted aryl esters, silyl esters, activated esters (e.g., derivatives of nitrophenol, pentafluorophenol, N-hydroxylsuccinimide, hydroxybenzotriazole, etc.), orthoesters, and the like. Examples of such ester groups include methyl, ethyl, propyl, isopropyl, butyl, isobutyl, tert-butyl, benzyl, and phenyl wherein each group is optionally substituted.

In certain aspects, functional groups that can be reacted to form carboxylic acids include, but are not limited to, amides, hydrazides, oxazolines, alkyl halides, alkenes, alkynes, and nitriles. In certain embodiments, group G is an alkene and the compound of formula E or E-a is oxidized to form carboxylic acid compound F-3 or F-3-a. The oxidation of the compound of formula E or E-a can be performed using known oxidation cleavage conditions, such as by using potassium permanganate, ozone/hydrogen peroxide, or ruthenium (III) chloride/sodium periodate. In certain embodiments, the oxidation of the compound of formula E or E-a is performed using ruthenium (III) chloride/sodium periodate. In certain embodiments, the oxidative cleavage of a compound of formula E or E-a can provide a compound of formula F-3 or F-3-a with various chain lengths of L¹. For example, oxidatation of a compound of formula E or E-a where -L^{1′}-G is

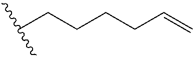 [SMILES: *CCCCC=C]

can provide a compound of formula F-3 or F-3-a wherein -L¹-CO₂H can include

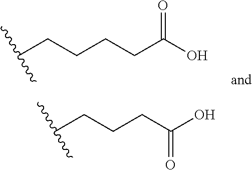 [SMILES: *CCCCC(=O)O ; "and" ; *CCCC(=O)O]

due to double bond migration, as discussed herein. Thus, in some embodiments, the compounds of the present invention may include or may be prepared from mixtures of oxidative cleavage products. Such mixtures may include from the smallest quantifiable amount by standard analysis methods (e.g., LCMS) to about a 50% mixture of oxidative cleavage products or downstream compounds derived therefrom.

In certain embodiments, the compounds of the current disclosure and the methods that include them comprise GalNAc as the beta anomer. In other embodiments, GalNAc is the alpha anomer. In some embodiments, GalNAc is a mixture of the beta anomer and the alpha anomer.

According to another aspect, the present invention provides a method for preparing a compound of formula F-5:

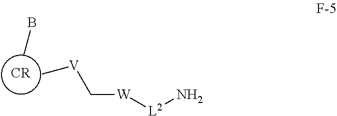

or a salt thereof, comprising the steps of:
(a) providing a compound of formula F-4:

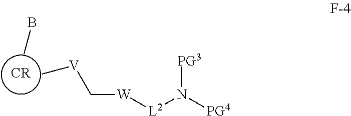

or a salt thereof, and
(b) deprotecting said fragment compound of formula F-4 to form the fragment compound of formula F-5, wherein:

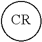

is

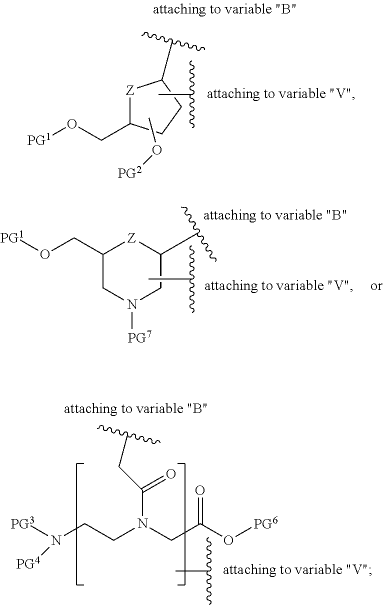

PG¹ and PG² are independently hydrogen or a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

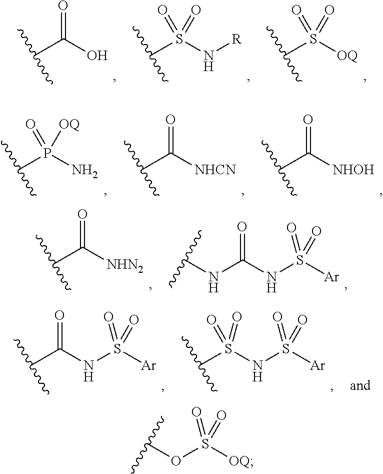

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to another aspect, the present invention provides a method for preparing a compound of formula F-5-a:

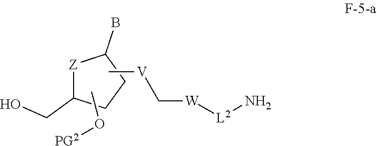

or a salt thereof, comprising the steps of:
(a) providing a compound of formula F-4-a:

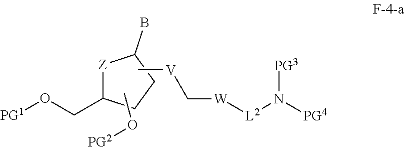

or a salt thereof, and
(b) deprotecting said fragment compound of formula F-4-a to form the fragment compound of formula F-5-a,
   wherein:
PG¹ and PG² are independently hydrogen or a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

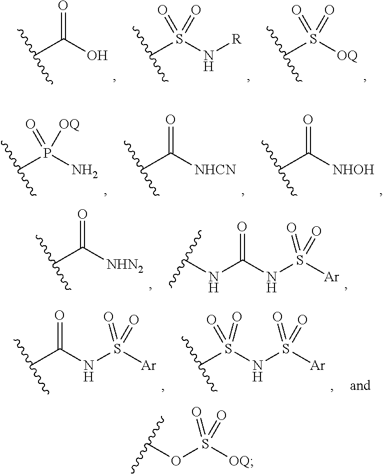

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to another aspect, the present invention provides a method for preparing a fragment compound of formula F-4:

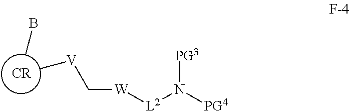

or a salt thereof, wherein:

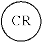

is

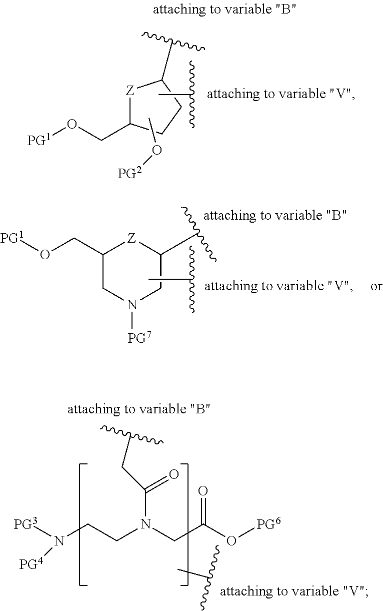

PG¹ and PG² are independently hydrogen or a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

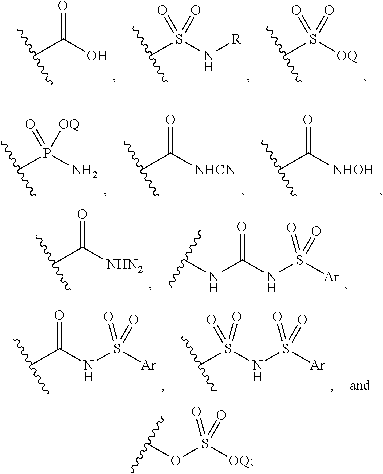

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—,
   comprising the steps of:
   (a) providing a fragment compound of formula F-1:

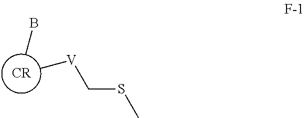

or a salt thereof, and

(b) alkylating said compound with a compound of formula F-2:

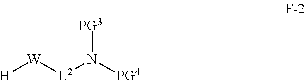

or a pharmaceutically acceptable salt thereof, to form a fragment compound of formula F-4.

According to another aspect, the present invention provides a method for preparing a fragment compound of formula F-4-a:

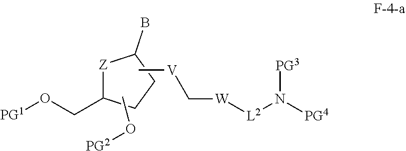

or a salt thereof, wherein:
PG¹ and PG² are independently hydrogen or a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

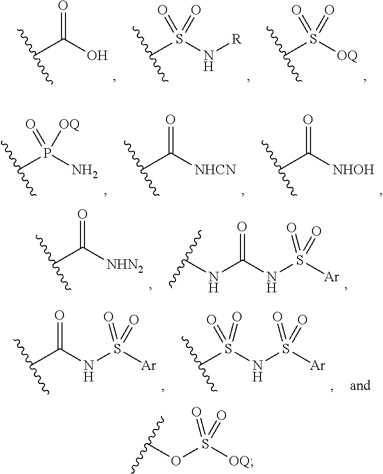

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—,
   comprising the steps of:
   (a) providing a fragment compound of formula F-1-a:

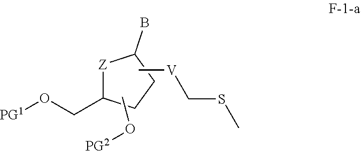

or a salt thereof, and
(b) alkylating said compound with a compound of formula F-2:

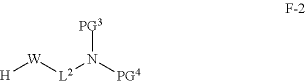

or a pharmaceutically acceptable salt thereof, to form a fragment compound of formula F-4-a.

According to one embodiment, step (b) above is performed under mild oxidizing and/or acidic conditions. In some embodiments, V is —O—. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

The PG³, PG⁴, and PG⁷ groups of the fragment compounds of formula F-2, F-4, and F-4-a are each independently hydrogen or a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of PG³, PG⁴, and PG⁷ groups of the fragment compounds of formula F-2, F-4, and F-4-a include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. In certain embodiments, PG³ and PG⁴ groups of the fragment compounds of formula F-2, F-4, and F-4-a do not include trifluoroacetyl.

In other embodiments, the PG³ and PG⁴ groups of the fragment compounds of formula F-2, F-4, and F-4-a are taken together with their intervening nitrogen atom to form a heterocyclic protecting group, such as phthalimide, pyrrole or pyrrolidine-2,5-dione. In certain embodiments, PG³ and PG⁴ groups of the fragment compounds of formula F-2, F-4, and F-4-a are not taken together with their intervening nitrogen to form phthalimide.

In certain embodiments, the PG³ group of the fragment compounds of formula F-2, F-4, and F-4-a is Fmoc and the PG⁴ group of the fragment compounds of formula F-2, F-4, and F-4-a is hydrogen, or vice versa.

Removal of protecting groups (e.g., both PG³ and PG⁴ or either of PG³ or PG⁴ independently from the same nitrogen) of the fragment compound of formula F-4 or F-4-a affords a fragment compound of formula F-5 or F-5-a or pharmaceutically acceptable salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) from the same nitrogen of the fragment compound of formula F-4 or F-4-a are removed by acid hydrolysis. It will be appreciated that upon acid hydrolysis of the protecting groups of the fragment compound of formula F-4 or F-4-a, a salt compound of the fragment compound of formula F-5 or F-5-a thereof is formed. For example, where an acid-labile protecting group of the fragment compound of formula F-4 or F-4-a is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a compound of formula F-5 or F-5-a are contemplated.

In other embodiments, the protecting groups (e.g., both PG³ and PG⁴ or either of PG³ or PG⁴ independently) from the same nitrogen of formula F-4 or F-4-a are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

In certain aspects, the present invention provides a method for preparing a fragment compound of formula F-5-a where the connectivity and stereochemistry is as shown in the fragment compound of formula F-5-b:

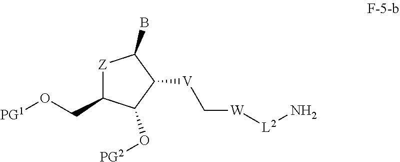

or a salt thereof, comprising the steps of:
(a) providing a fragment compound of formula F-4-b:

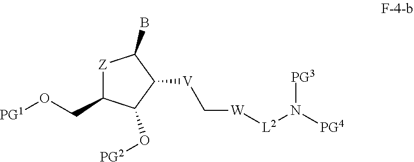

or a salt thereof, and
(b) deprotecting said fragment compound of formula F-4-b to form a fragment compound of formula F-5-b,
wherein each of PG¹, PG², PG³, PG⁴, B, L², V, W, and Z is as defined and in classes and subclasses as described herein.

In certain aspects, the present invention provides a method for preparing a fragment compound of formula F-4-a where the connectivity and stereochemistry is as shown in the fragment compound of formula F-4-b:

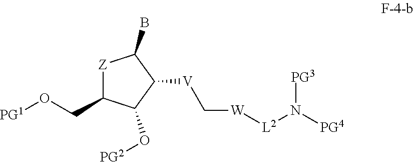

or a salt thereof, comprising the steps of:
(a) providing a fragment compound of formula F-1-b:

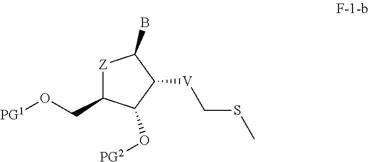

or a salt thereof, and
(b) alkylating said compound with a compound of formula F-2:

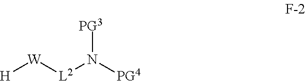

or a salt thereof,
to form a fragment compound of formula F-4-b,
wherein each of PG¹, PG², PG³, PG⁴, B, L², V, W, and Z is as defined and in classes and subclasses as described herein.

According to another aspect, the present invention provides a method for preparing a fragment compound of formula F-1:

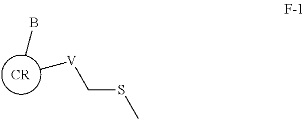

or a salt thereof, wherein

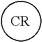

is

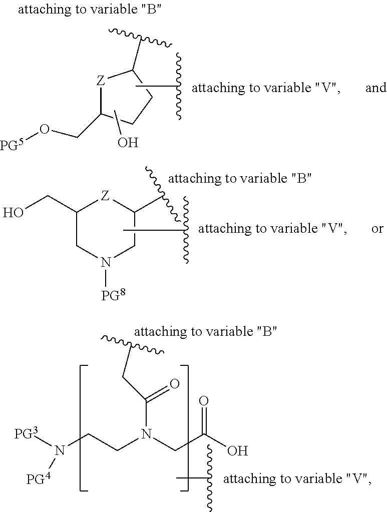

comprising the steps of:
(a) providing a compound of formula J:

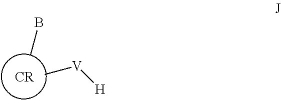

or a salt thereof, wherein

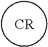

is

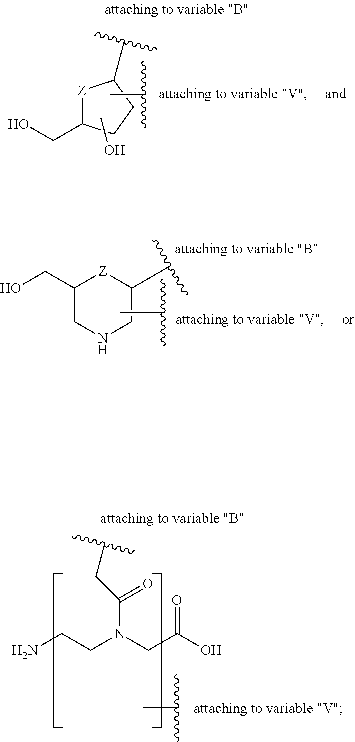

(b) protecting said compound of formula J with suitable protecting groups to form a compound of formula I:

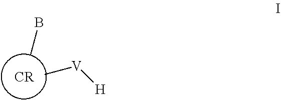

or a salt thereof, wherein

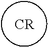

is

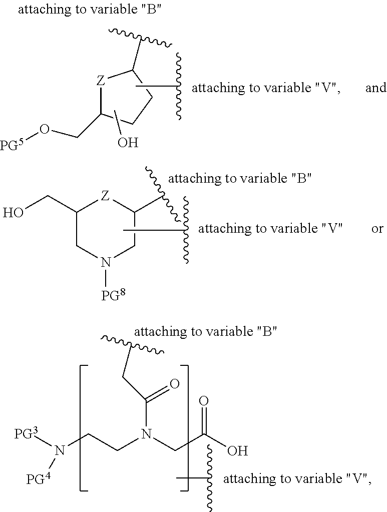

(c) alkylating said compound of formula I to form a compound of formula F-1, wherein:
PG¹ and PG² are independently hydrogen or a suitable hydroxyl protecting group;
PG³, PG⁴, and PG⁷ are independently hydrogen or a suitable nitrogen protecting group;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

According to another aspect, the present invention provides a method for preparing a fragment compound of formula F-1-a:

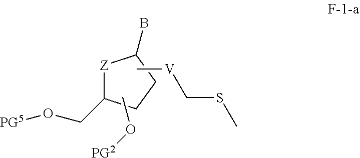

or a salt thereof, comprising the steps of:
(a) providing a compound of formula J-a:

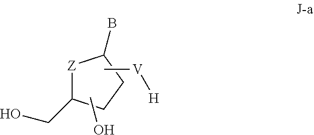

or a salt thereof, and
(b) protecting said compound of formula J with suitable protecting groups to form a compound of formula I:

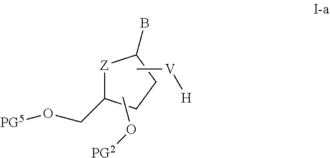

or a salt thereof, and
(c) alkylating said compound of formula I-a to form a compound of formula F-1-a, wherein:
PG¹ and PG² are independently hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

According to one embodiment, protecting a compound of formula J or J-a in step (b) above includes the use of suitable hydroxyl protecting groups and in some instances suitable nitrogen protecting groups. Suitable hydroxyl protecting groups are well known in the art and are described in detail above. In some embodiments, PG¹ and PG² are protected using cyclic diol protection group. In certain embodiments, the cyclic diol protection group is 1,1,3,3-tetraisopropylidisiloxanylidene prepared from the reaction of a diol of formula J or J-a and 1,3-dichloro-1,1,3,3-tetraisopropyldisiloxane under basic conditions. One of ordinary skill would recognize that the displacement of a leaving group in a protecting group reagent by the hydroxyl moieties of a compound of formula J or J-a is achieved either with or without the presence of a suitable base. Such suitable bases are well known in the art and include organic and inorganic bases. In certain embodiments, the base is a tertiary amine such as triethylamine or diisopropylethylamine. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of the PG³ group used to protect a compound of formula J or J-a in step (b) above include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

According to another embodiment, the alkylation at step (c) above is achieved by reacting a compound of formula I or I-a with a mixture of DMSO and acetic anhydride under acidic conditions. In certain embodiments, when V—H is a hydroxyl group, the mixture of DMSO and acetic anhydride in the presence of acetic acid forms (methylthio)methyl acetate in situ via the Pummerer rearrangement which then reacts with the hydroxyl group of the compound of formula I or I-a to provide a monothioacetal functionalized fragment compound of formula F-1 or F-1-a. In certain embodiments, the alkylation is achieved using an organic acid, such as acidic acid at an elevated temperature, e.g., about 30° C. to about 70° C.

In certain aspects, the present invention provides a method for preparing a fragment compound of formula F-1-a where the connectivity and stereochemistry is as shown in the compound of formula F-1-b:

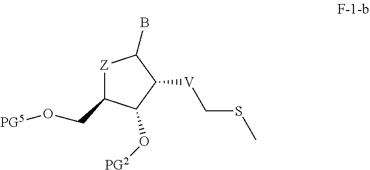

or a salt thereof, comprising the steps of:
(a) providing a compound of formula J-b:

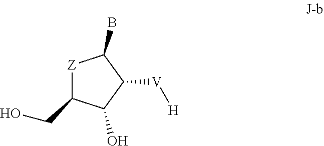

or a salt thereof,
(b) protecting said compound of formula J-b with suitable protecting groups to form a compound of formula I-b:

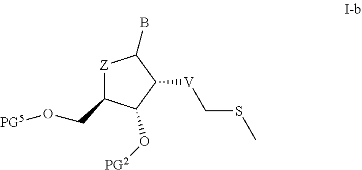

or a salt thereof, and
(c) alkylating said compound of formula I-b to form a fragment compound of formula F-1-b, wherein each of PG¹, PG², B, V, and Z is as defined and in classes and subclasses as described herein.

According to another aspect, the present invention provides a method for preparing a compound of formula F-6:

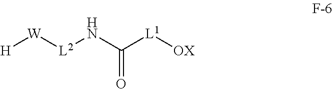

or a salt thereof, comprising the steps of:
(a) providing a fragment compound of formula F-3:

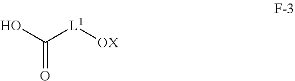

or a salt thereof, and
(b) reacting said fragment compound of formula F-3 with a fragment compound of formula F-2:

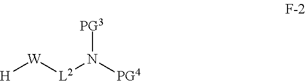

or a salt thereof,
to form the fragment compound of formula F-6, wherein:
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

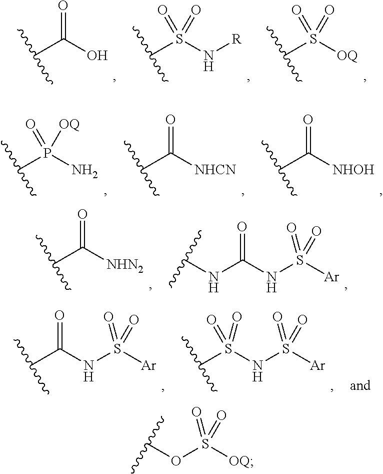

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
X is a ligand selected from GalNAc, D-mannose, L-galactose, D-arabinose, L-fucose, polyols, and

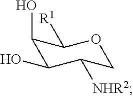

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl;
PG³ and PG⁴ are independently hydrogen; and
W is —O—, —S—, or —NR—.

In certain embodiments, reacting said fragment compound of formula F-3 with the fragment compound of formula F-2 above comprises an amidation reaction. In certain embodiments, the amidation reaction is achieved under suitable amide forming conditions.

In some embodiments, the amidation reaction can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of compound of formula F-3 is converted to an activated ester, followed by reacting with an amine compound. In certain embodiments, the activated ester forming conditions include a mixture of NHS (N-hydroxysuccinimide and EDC [1-ethyl-3-(3-dimethilaminopropyl)carbodiimide].

In certain alternative aspects, the present invention provides a method for preparing a fragment compound of formula F-6 where X is GalNAc and the connectivity and stereochemistry is as shown in the fragment compound of formula F-6-a:

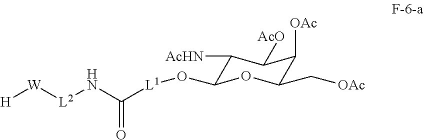

or a salt thereof, comprising the steps of:
(a) providing a fragment compound of formula F-3-a:

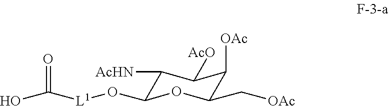

or a salt thereof, and
(b) reacting said fragment compound of formula F-3-a with a fragment compound of formula F-2:

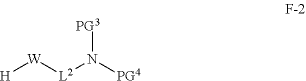

or a salt thereof,
to form the fragment compound of formula F-6-a, wherein each of L¹, L², and W is as defined and in classes and subclasses as described herein, and PG³ and PG⁴ are independently hydrogen.

According to another alternative aspect, the present invention provides a method for preparing a compound of formula D:

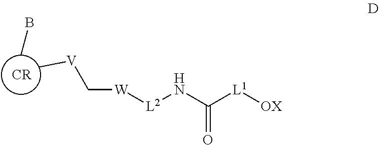

or a salt thereof, wherein:

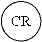

is

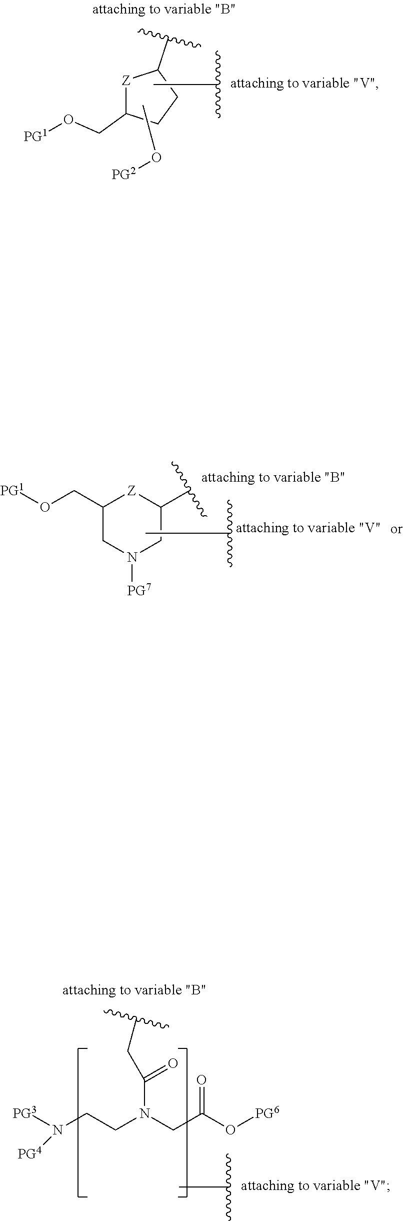

PG¹ and PG² are independently hydrogen or a suitable hydroxyl protecting group;
PG³, PG⁴, and PG⁷ are independently hydrogen or a suitable nitrogen protecting group;
PG⁶ is hydrogen or a suitable carboxylate protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

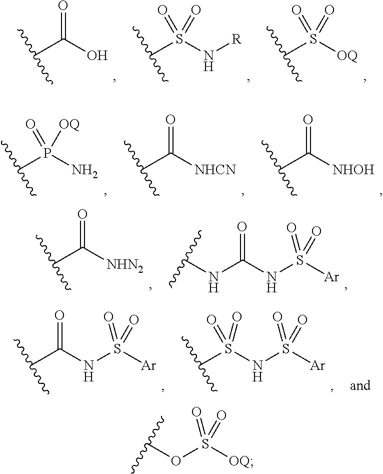

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

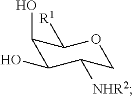

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula F-1:

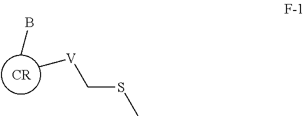

or a salt thereof, and
(b) reacting said fragment compound of formula F-1 with a fragment compound of formula F-6:

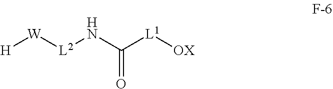

or a salt thereof, to provide the compound of formula D.

According to another alternative aspect, the present invention provides a method for preparing a compound of formula D-a:

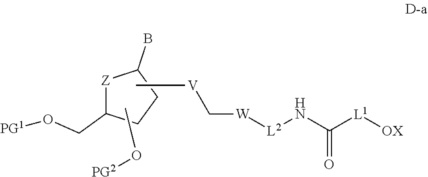

or a salt thereof, wherein:
PG¹ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

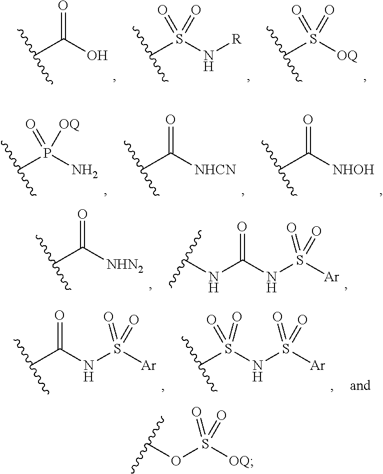

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

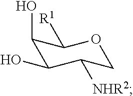

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula F-1-a:

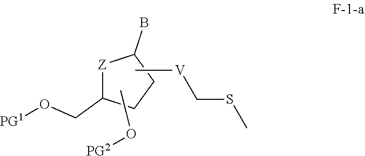

or a salt thereof, and
(b) reacting said fragment compound of formula F-1-a with a fragment compound of formula F-6:

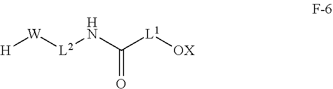

or a salt thereof,
to provide the compound of formula D-a.

According to one embodiment, step (b) above is performed under mild oxidizing and/or acidic conditions. In some embodiments, V is —O—. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In certain alternative aspects, the present invention provides a method for preparing a compound of formula D-a where X is GalNAc and the connectivity and stereochemistry is as shown in the compound of formula D-b:

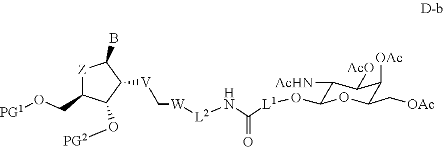

or a salt thereof, comprising the steps of:
(a) providing a compound of formula F-1-b:

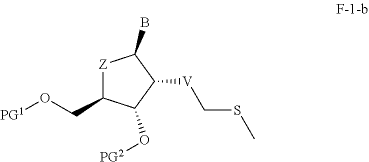

or a salt thereof, and
(b) reacting said fragment compound of formula F-1-b with a fragment compound of formula F-6-a:

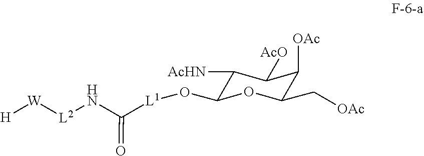

or a salt thereof,
to provide the compound of formula D-b, wherein each of PG¹, PG², B, L¹, L², V, W, and Z is as defined and in classes and subclasses as described herein.

According to an alternative aspect, the present invention provides a method for preparing a compound of formula N1:

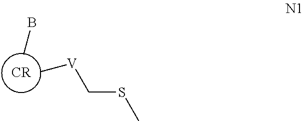

or a salt thereof, wherein:

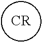

is

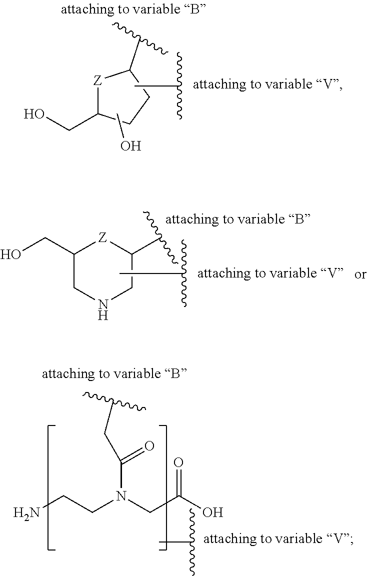

B is a nucleobase or hydrogen;
V and W are independently —O—, —S—, or —NR—;
each R is independently selected from hydrogen, alkyl, alkenyl, aromatic, heterocycle, substituted alkyl, substituted alkenyl;
Z is —CH₂—, —O—, —S—, or —NR—,
   comprising the steps of:
   (a) providing a compound of formula F-1:

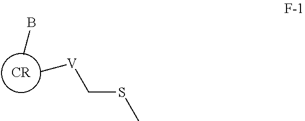

or a salt thereof, wherein:

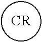

is

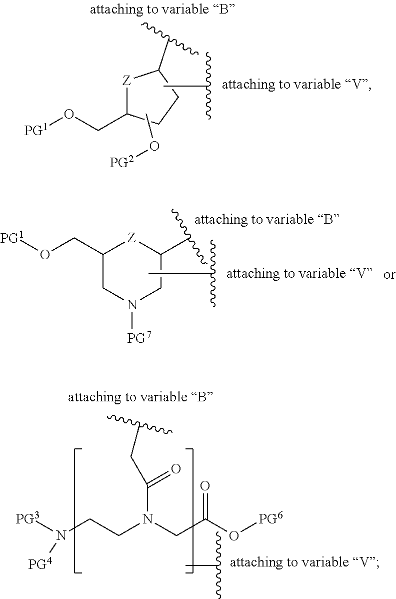

PG¹ and PG² are independently a suitable hydroxyl protecting group;
PG³, PG⁴, and PG⁷ are independently hydrogen or a suitable nitrogen protecting group;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—, and
   (b) deprotecting said compound of formula F-1 to form a compound of formula N1.

According to an alternative aspect, the present invention provides a method for preparing a compound of formula N1-a:

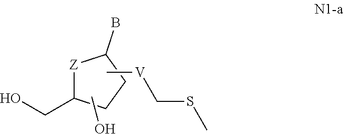

or a salt thereof, wherein:
B is a nucleobase or hydrogen;
V and W are independently —O—, —S—, or —NR—;
each R is independently selected from hydrogen, alkyl, alkenyl, aromatic, heterocycle, substituted alkyl, substituted alkenyl;
Z is —CH₂—, —O—, —S—, or —NR—,
   comprising the steps of:
   (a) providing a compound of formula F-1-a:

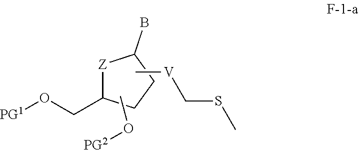

or a salt thereof, wherein:
PG¹ and PG² are independently hydrogen or a suitable hydroxyl protecting group;
B is a nucleobase or hydrogen;
V is —O—, —S—, or —NR—;
   each R is independently selected from hydrogen, alkyl, alkenyl, aromatic, heterocycle, substituted alkyl, and substituted alkenyl; and
Z is —CH₂—, —O—, —S—, or —NR—, and
   (b) deprotecting said compound of formula F-1-a to form a compound of formula N1-a.

According to one embodiment, PG¹, PG², and PG³ removed in step (b) above are selected from suitable hydroxyl protecting groups and suitable nitrogen protection groups. Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, 0-nitrobenzyl, p-nitrobenzyl, p-halobenzyl, 2,6-dichlorobenzyl, p-cyanobenzyl, and 2- and 4-picolyl.

In certain embodiments, the PG¹ and PG² groups removed to form a compound of formula F-1 in step (b) above are taken together to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene. In some embodiments, 1,1,3,3-tetraisopropylidisiloxanylidene is removed under acidic conditions or with fluoride anion. Examples of acids for the removal of silicon-based protecting groups include suitable acids well known in the art such as inorganic acids, e.g., hydrochloric acid, hydrobromic acid, phosphoric acid, nitric acid, sulfuric acid or perchloric acid, or organic acids, e.g., acetic acid, trifluoroacetic acid, p-toluenesulfonic acid, or methanesulfonic acid. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

Suitable amino protecting groups are well known in the art and include those described in detail in Protecting Groups in Organic Synthesis, T. W. Greene and P. G. M. Wuts, 3P edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of the PG³ group deprotected in step (b) above include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

According to another alternative aspect, the present invention provides a method for preparing a compound of formula N2:

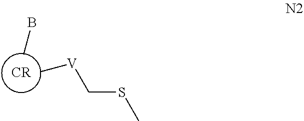

or a salt thereof, wherein:

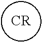

is

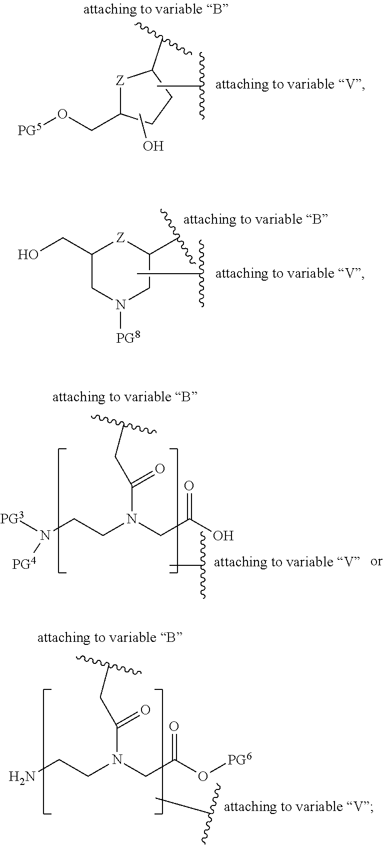

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—,
   comprising the steps of:
(a) providing a compound of formula N1:

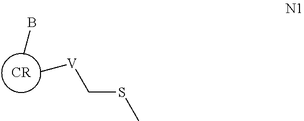

or a salt thereof, wherein:

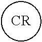

is

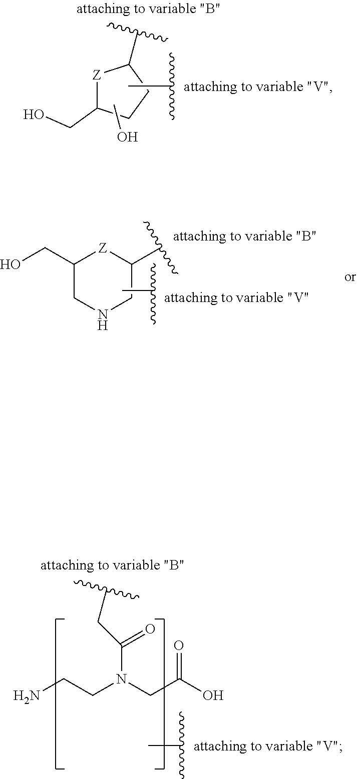

B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—, and
   comprising the steps of:
(b) protecting said compound of formula N1 with a suitable protecting group to form a compound of formula N2.

In certain embodiments, the protecting group PG⁸ used for selective protection of a nitrogen group, for example, in formulas N2 and N3, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another alternative aspect, the present invention provides a method for preparing a compound of formula N2-a:

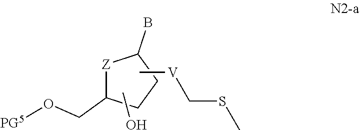

or a salt thereof, wherein:
PG⁵ is hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—,
   comprising the steps of:
(a) providing a compound of formula N1-a:

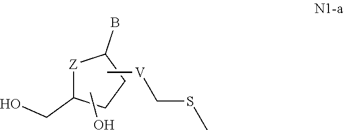

or a salt thereof, wherein:
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—, and
   comprising the steps of:
(b) protecting said compound of formula N1-a with a suitable protecting group to form a compound of formula N2-a.

According to one embodiment, a compound of formula N1 or N1-a in selectively protected in step (b) above with a suitable protecting group. In some embodiments, the protecting group PG⁵ used for the selective protection of the 5′-hydroxyl group of a compound of formula N1 or N1-a or in some instances the lone hydroxyl group of a compound of formula N1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another alternative aspect, the present invention provides a method for preparing a compound of formula N3:

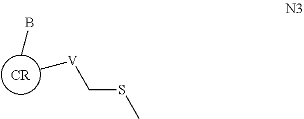

or a salt thereof, wherein:

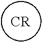

is

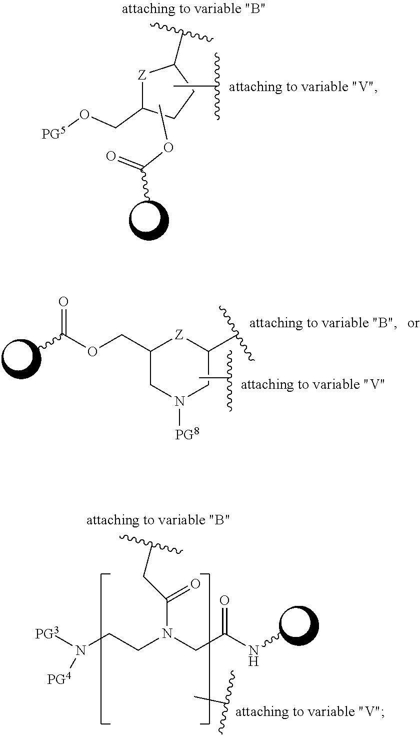

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—,
   comprising the steps of:
(a) providing a solid support of formula

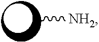

and a compound of formula N2:

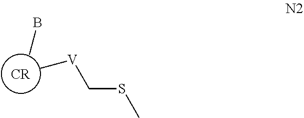

or a salt thereof, wherein:

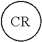

is

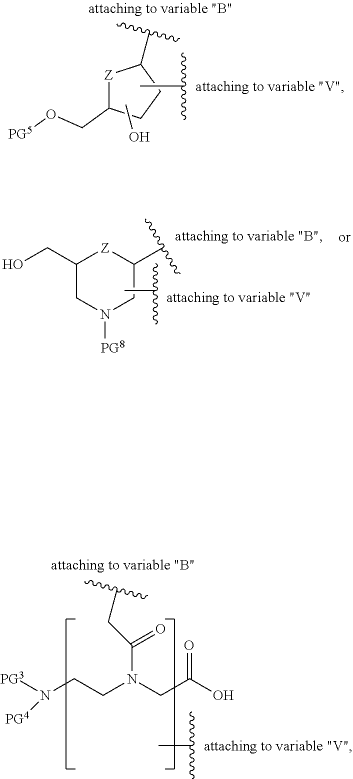

and
(b) reacting said compound of formula N2 with the solid support of formula

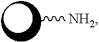

to form a compound of formula N3.

According to another alternative aspect, the present invention provides a method for preparing a compound of formula N3-a:

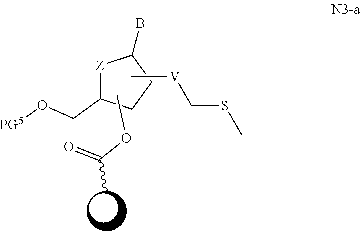

or a salt thereof, wherein:
PG⁵ is hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—,
   comprising the steps of:
   (a) providing a solid support of formula

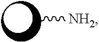

and a compound of formula N2-a:

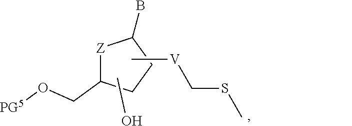

and
(b) reacting said compound of formula N2-a with the solid support of formula

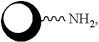

to form a compound of formula N3-a.

In certain embodiments, the hydroxyl group of a compound of formula N2 or N2-a or in some instance the nitrogen of a compound of formula N2 is covalently attached to a solid support through a succinic acid linking group. One of ordinary skill would recognize that the covalent attachment of a compound of formula N2 or N2-a to a solid support could be performed by reacting with a dicarboxylic acid compound, or an anhydride thereof, forming an ester with the —OH of the compound of formula N2 or N2-a and an amide with the —NH₂ of the solid support. Formation of esters appropriate for solid support synthesis are well known in the art, e.g., see, “Advanced Organic Chemistry”, Jerry March, 5^{th} edition, John Wiley and Sons, N.Y.

According to another aspect, the present invention provides a method for preparing a compound of formula A1:

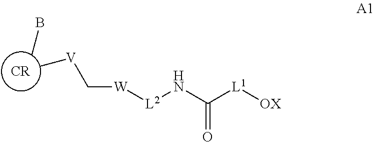

or a salt thereof, wherein:

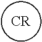

is

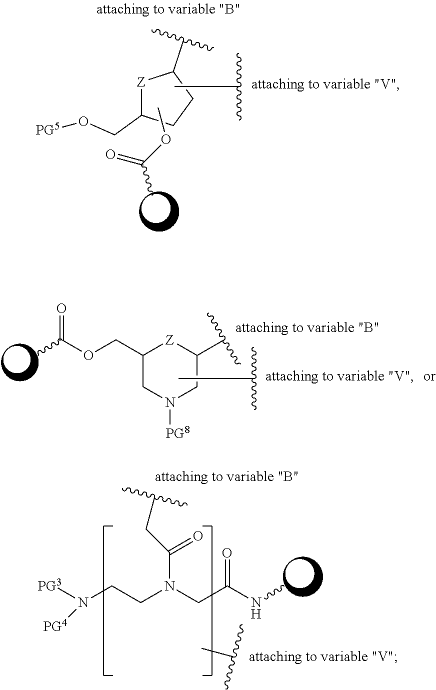

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

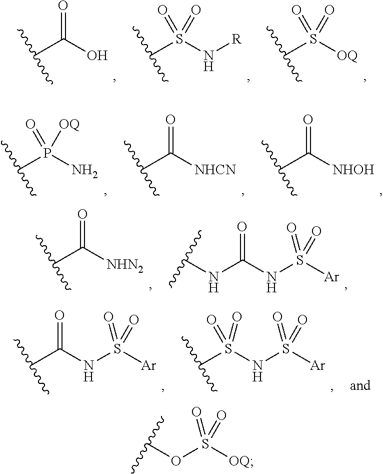

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

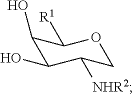

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula N3:

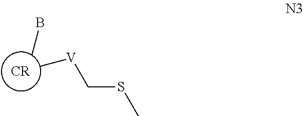

or a salt thereof, and
(b) reacting said fragment compound of formula N3 with a fragment compound of formula F-6:

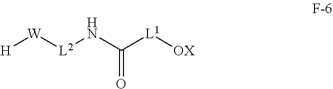

or a salt thereof, to provide the compound of formula A1.

According to another aspect, the present invention provides a method for preparing a compound of formula A1-a:

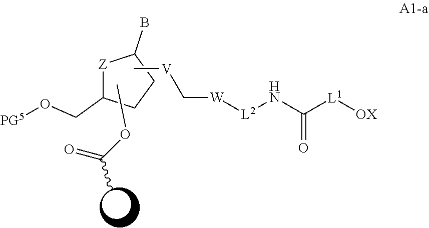

or a salt thereof, wherein:
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

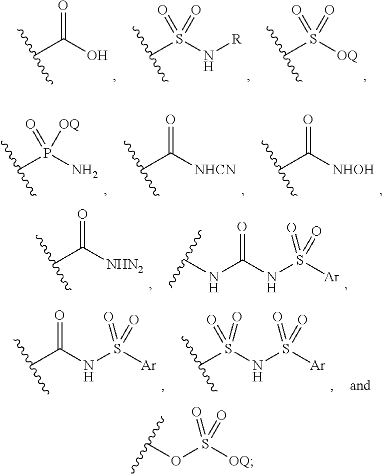

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

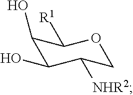

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
   comprising the steps of:
(a) providing a compound of formula N3-a:

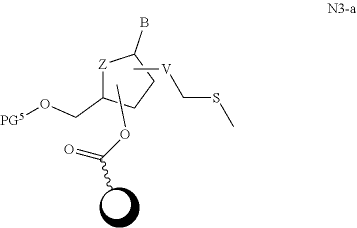

or a salt thereof, comprising the steps of
(b) reacting said fragment compound of formula N3-a with a fragment compound of formula F-6:

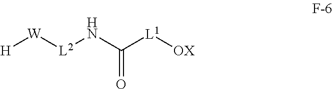

or a salt thereof, to provide the compound of formula A1.

According to one embodiment, step (b) above is performed under mild oxidizing and/or acidic conditions. In some embodiments, V is —O—. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

According to another alternative aspect, the present invention provides a method for preparing a compound of formula M1:

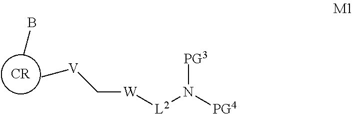

or a salt thereof, wherein

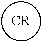

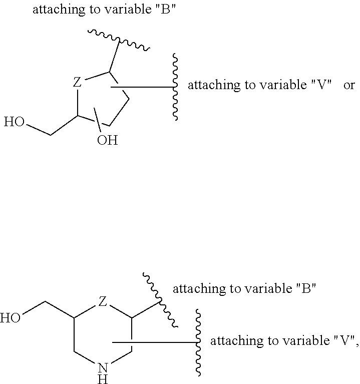

comprising the steps of:
(a) providing a compound of formula F-4:

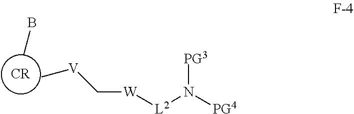

or a salt thereof, wherein

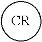

is

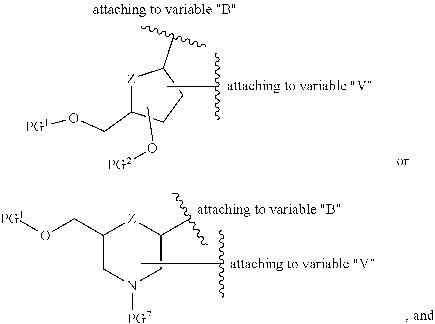

(b) deprotecting said fragment compound of formula F-4 to form a compound of formula M1, wherein:
B is a nucleobase or hydrogen;
PG¹ and PG² are independently a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

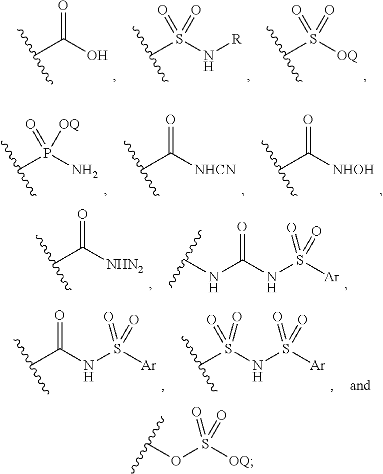

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to another alternative aspect, the present invention provides a method for preparing a compound of formula M1-a:

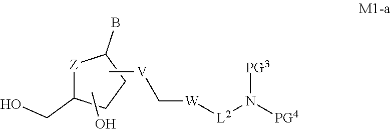

or a salt thereof, comprising the steps of:
(a) providing a compound of formula F-4-a:

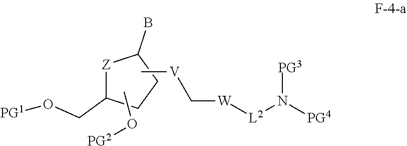

or a salt thereof, and
(b) deprotecting said fragment compound of formula F-4-a to form a compound of formula M1-a, wherein:
PG¹ and PG² are independently a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
PG³ and PG⁴ are independently hydrogen or a suitable nitrogen protecting group, provided both PG³ and PG⁴ are not hydrogen at the same time;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

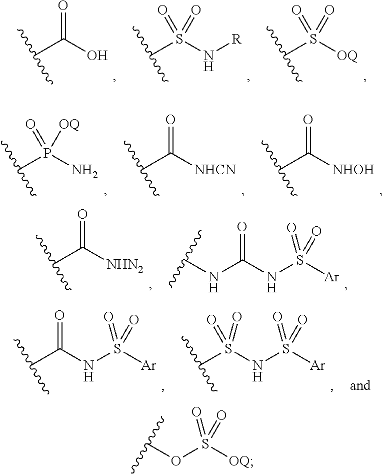

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to one embodiment, PG¹, PG², and PG³ removed in step (b) above are selected from suitable hydroxyl protecting groups and suitable nitrogen protection groups.

Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

In certain embodiments, the PG¹ and PG² groups removed to form a fragment compound of formula F-4 or F-4-a in step (b) above are taken together to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene. In some embodiments, 1,1,3,3-tetraisopropylidisiloxanylidene is removed under acidic conditions or with fluoride anion. Examples of acids for the removal of silicon-based protecting groups include suitable acids well known in the art such as inorganic acids, e.g., hydrochloric acid, hydrobromic acid, phosphoric acid, nitric acid, sulfuric acid or perchloric acid, or organic acids, e.g., acetic acid, trifluoroacetic acid, p-toluenesulfonic acid, or methanesulfonic acid. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

Suitable amino protecting groups are well known in the art and include those described in detail in Protecting Groups in Organic Synthesis, T. W. Greene and P. G. M. Wuts, 3d edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of the PG³ group deprotected in step (b) above include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

According to another aspect, the present invention provides a method for preparing a compound of formula M2:

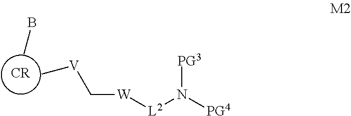

or a salt thereof, wherein

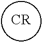

is

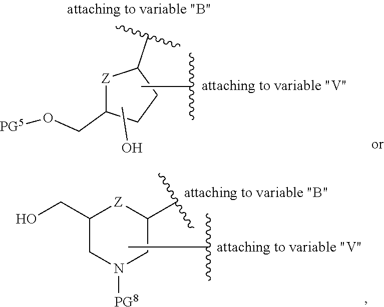

comprising the steps of:
(a) providing a compound of formula M1:

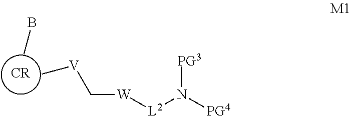

or a salt thereof, wherein

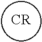

is

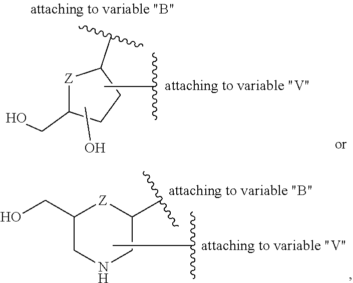

(b) protecting said compound of formula M1 with a suitable protecting group to form a compound of formula M2,
   wherein:
PG³, PG⁴, and PG⁸ are independently hydrogen or a suitable nitrogen protecting group, provided both PG³ and PG⁴ are not hydrogen at the same time;
PG⁵ is a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

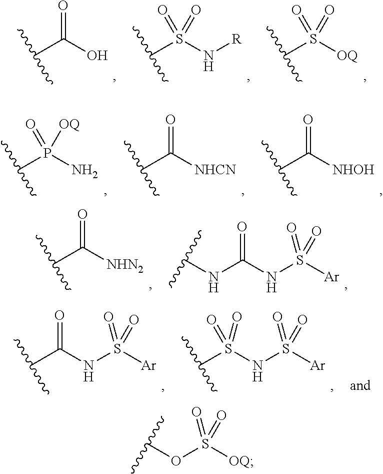

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

According to another aspect, the present invention provides a method for preparing a compound of formula M2-a:

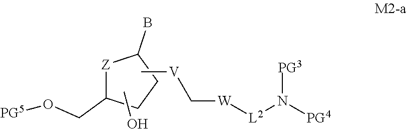

or a salt thereof,
comprising the steps of:
(a) providing a compound of formula M1-a:

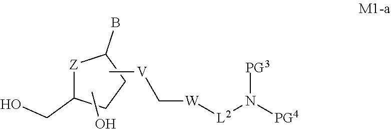

or a salt thereof, and
(b) protecting said compound of formula M1-a with a suitable protecting group to form a compound of formula M2-a,
   wherein:
PG³ and PG⁴ are independently hydrogen or a suitable nitrogen protecting group, provided both PG³ and PG⁴ are not hydrogen at the same time;
PG⁵ is a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

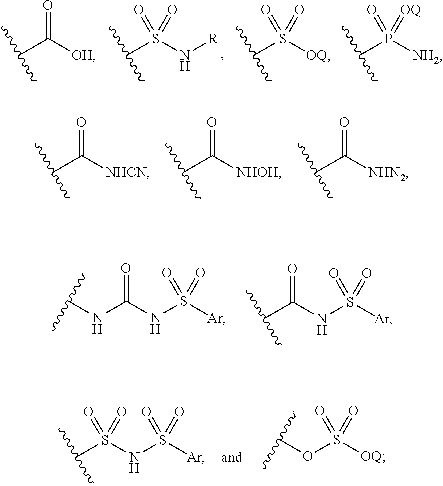

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V is —O—, —S—, or —NR—;
   each R is independently selected from hydrogen, alkyl, alkenyl, aromatic, heterocycle, substituted alkyl, and substituted alkenyl; and
Z is —CH₂—, —O—, —S—, or —NR—.

According to one embodiment, a compound of formula M1 or M1-a is selectively protected in step (b) above with a suitable protecting group. In some embodiments, the protecting group PG⁵ used for the selective protection of the 5′-hydroxyl group of a compound of formula M1 or M1-a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another aspect, the present invention provides a method for preparing a compound of formula M3:

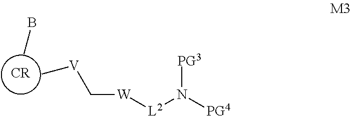

or a salt thereof, wherein

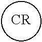

is

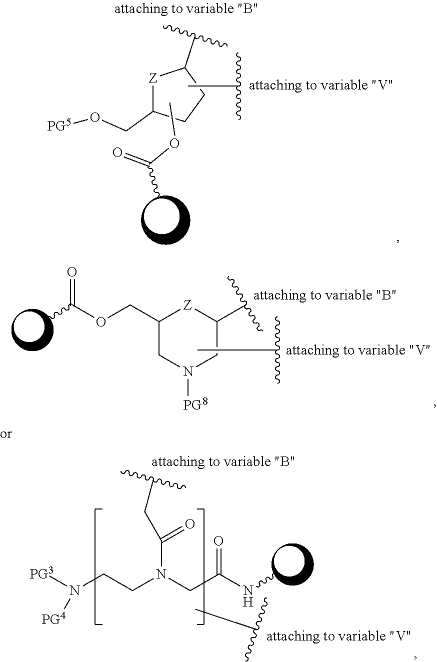

comprising the steps of:
(a) providing a solid support of formula

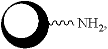

and a compound of formula M2:

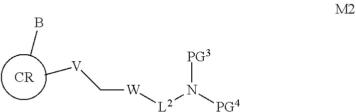

or a salt thereof, wherein

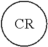

is

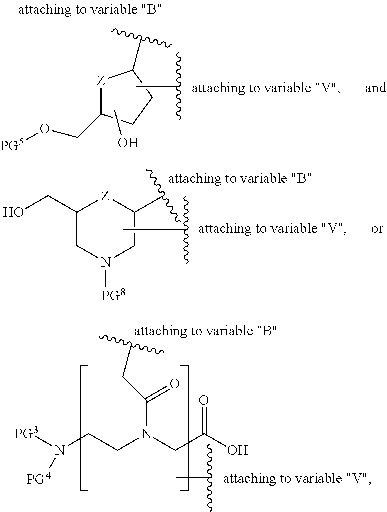

(b) reacting said compound of formula M2 with the solid support of formula

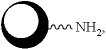

to form a compound of formula M3, wherein:
B is a nucleobase or hydrogen;
PG³, PG⁴, and PG⁸ are independently hydrogen or a suitable nitrogen protecting group, provided both PG³ and PG⁴ are not hydrogen at the same time;
PG⁵ is hydrogen or a suitable hydroxyl protecting group;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

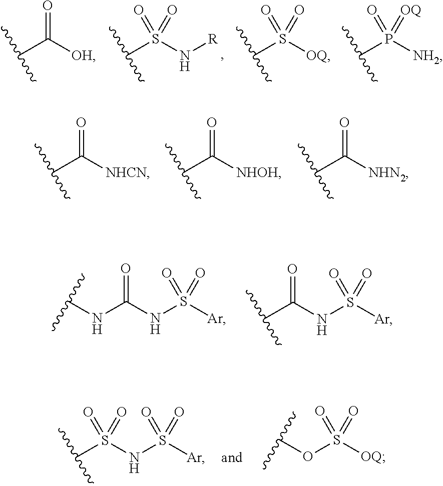

each R is independently selected from hydrogen, alkyl, alkenyl, aromatic, heterocycle, substituted alkyl, substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to another aspect, the present invention provides a method for preparing a compound of formula M3-a:

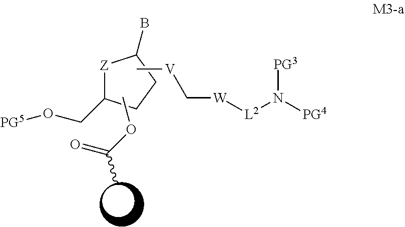

or a salt thereof, comprising the steps of:
(a) providing a solid support of formula

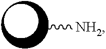

and a compound of formula M2-a

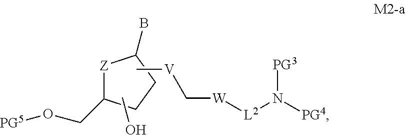

(b) reacting said compound of formula M2-a with the solid support of formula

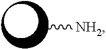

to form a compound of formula M3-a, wherein:
B is a nucleobase or hydrogen;
PG³ and PG⁴ are independently hydrogen or a suitable nitrogen protecting group, provided both PG³ and PG⁴ are not hydrogen at the same time;
PG⁵ is hydrogen or a suitable hydroxyl protecting group;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

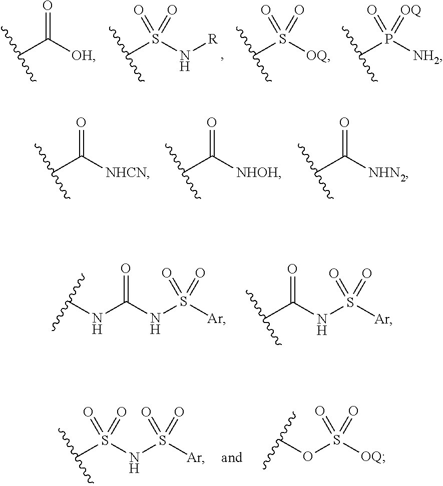

each R is independently selected from hydrogen, alkyl, alkenyl, aromatic, heterocycle, substituted alkyl, substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the hydroxyl group of a compound of formula M2 or M2-a or the nitrogen group of a compound of formula M2 is covalently attached to a solid support through a succinic acid linking group. One of ordinary skill would recognize that the covalent attachment of a compound of formula M2 or M2-a to a solid support could be performed by reacting with a dicarboxylic acid compound, or an anhydride thereof, forming an ester with the —OH of the compound of formula M2 or M2-a and an amide with the —NH₂ of the solid support. Formation of esters appropriate for solid support synthesis are well known in the art, e.g., see, “Advanced Organic Chemistry”, Jerry March, 5^{th} edition, John Wiley and Sons, N.Y.

According to alternate aspect, the present invention provides a method for preparing a compound of formula M4:

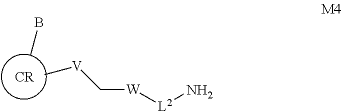

or a salt thereof, comprising the steps of:
(a) providing a compound of formula M3:

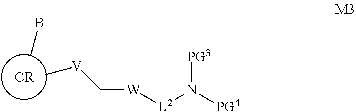

or a salt thereof, and
(b) deprotecting said fragment compound of formula M3 to form the fragment compound of formula M4, wherein:

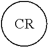

is

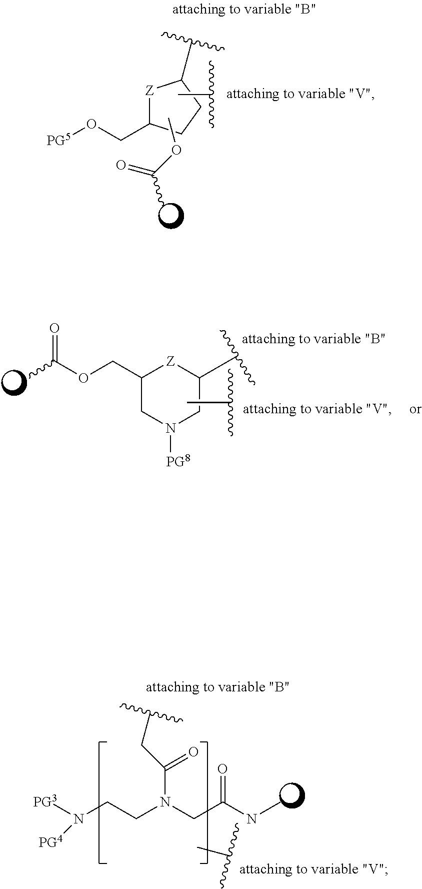

PG³, PG⁴, and PG⁸ are independently a hydrogen or a suitable nitrogen protecting group, provided both PG³ and PG⁴ are not hydrogen at the same time;
PG⁵ is hydrogen or a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

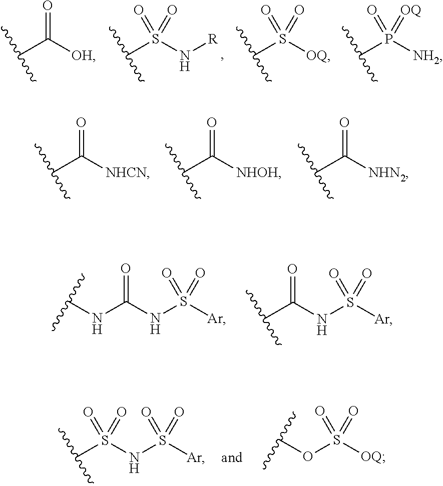

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the protecting group PG⁸ used for selective protection of a nitrogen group, for example, in formulas M2, M3, and M4, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to alternate aspect, the present invention provides a method for preparing a compound of formula M4-a:

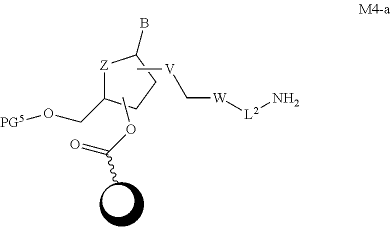

or a salt thereof, comprising the steps of
(a) providing a compound of formula M3-a:

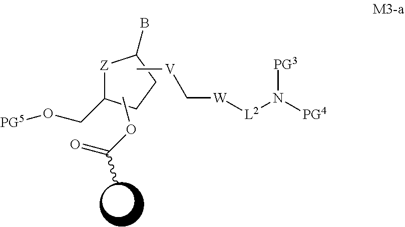

or a salt thereof, and
(b) deprotecting said fragment compound of formula M3-a to form the fragment compound of formula M4-a, wherein:
PG⁵ is hydrogen or a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

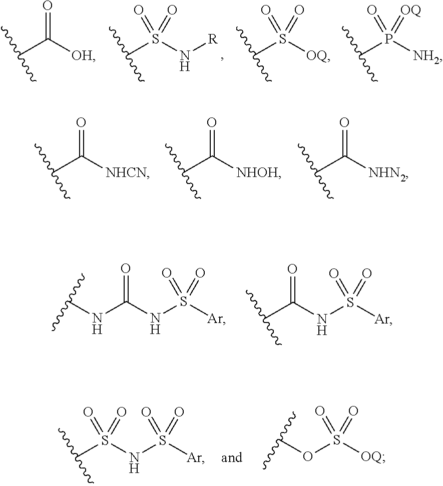

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

The PG³ and PG⁴ groups of the compound of formula M3 or M3-a are each independently hydrogen or a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of PG³ and PG⁴ groups of the compound of formula M3 or M3-a include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. In other embodiments, the PG³ and PG⁴ groups of the compound of formula M3 or M3-a are taken together with their intervening nitrogen atom to form a heterocyclic protecting group, such as a pyrrole or pyrrolidine-2,5-dione.

Removal of protecting groups (e.g., both PG³ and PG⁴ or either of PG³ or PG⁴ independently) of the compound of formula M3 or M3-a affords a compound of formula M4 or M4-a or salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) of the compound of formula M3 or M3-a are removed by acid hydrolysis. It will be appreciated that upon acid hydrolysis of the protecting groups of the compound of formula M3 or M3-a, a salt compound of the fragment compound of formula M4 or M4-a thereof is formed. One of ordinary skill in the art would recognize that a wide variety of acids are useful for removing amino protecting groups that are acid-labile and therefore a wide variety of salt forms of a compound of formula M4 or M4-a are contemplated.

In other embodiments, the protecting groups (e.g., both PG³ and PG⁴ or either of PG³ or PG⁴ independently) of formula M3 or M3-a are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

According to another alternative aspect, the present invention provides a method for preparing a compound of formula A1:

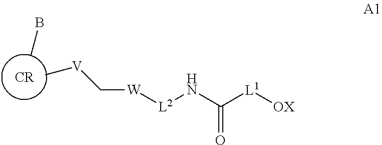

or a salt thereof, comprising the steps of:
(a) providing a compound of formula F-3:

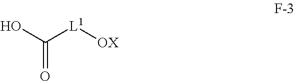

or a salt thereof, and
(b) reacting said fragment compound of formula F-3 with a fragment compound of formula M4:

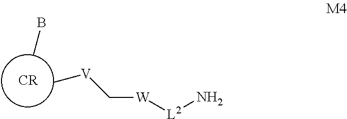

or a salt thereof, to provide the compound of formula A1, wherein:

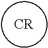

is

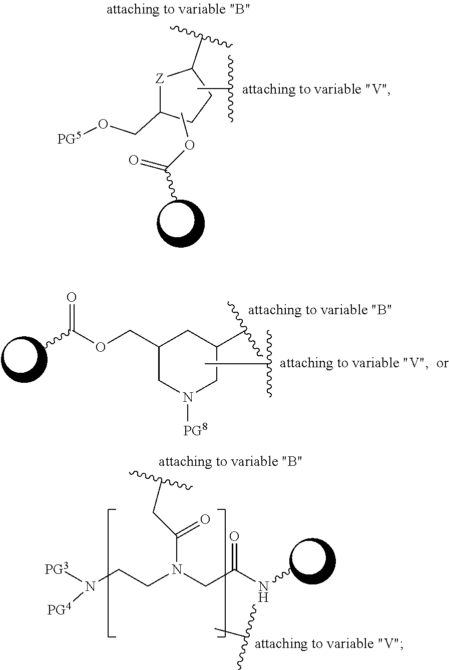

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

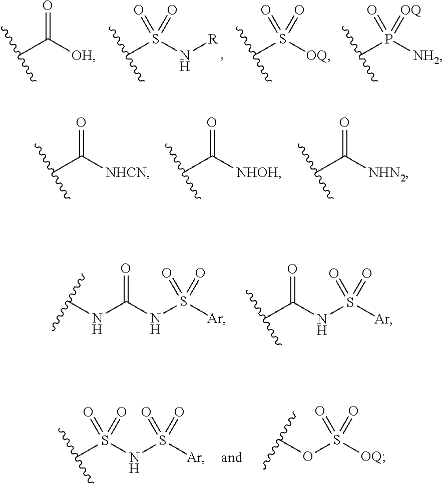

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

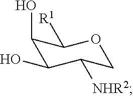

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to another alternative aspect, the present invention provides a method for preparing a compound of formula A1-a:

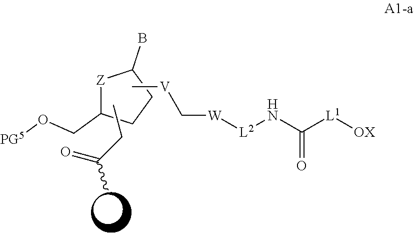

or a salt thereof, comprising the steps of:
(a) providing a compound of formula F-3:

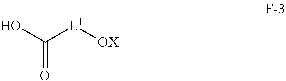

or a salt thereof, and
(b) reacting said fragment compound of formula F-3 with a fragment compound of formula M4-a:

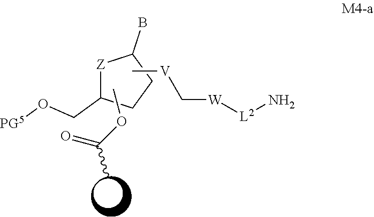

or a salt thereof,
to provide the compound of formula A1-a, wherein:
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

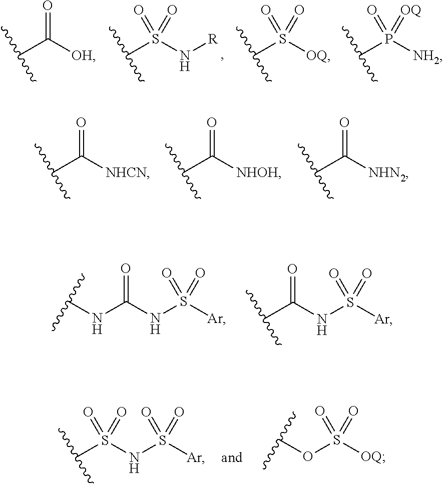

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

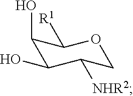

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to one embodiment, the amidation reaction of step (b) can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with an amine compound. In certain embodiments, the activated ester forming conditions include a mixture of NHS (N-hydroxysuccinimide and EDC [1-ethyl-3-(3-dimethylaminopropyl)carbodiimide].

Without being limited to the current disclosure, the assembly of fragment compound of formula F-3 with the solid-state compound of formula M4 or M4-a in step (b) could be facilitated using a range of cross-linking technologies. It is within the purview of those having ordinary skill in the art that the carboxylic acid of the fragment compound of formula F-3 and the amine of the solid state compound of formula M4 or M4-a could be replaced by suitable coupling moieties that react with each other to covalently link the fragment compound of formula F-3 with the solid state compound of formula M4 or M4-a by alternative means. Exemplary cross-linking technologies envisioned for use in the current disclosure also include those listed in Table 1 disclosed herein.

According to another aspect, the present invention provides a method for preparing a compound of formula P1:

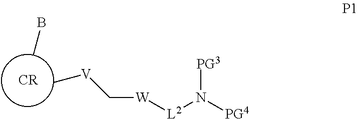

or a salt thereof, wherein

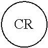

is

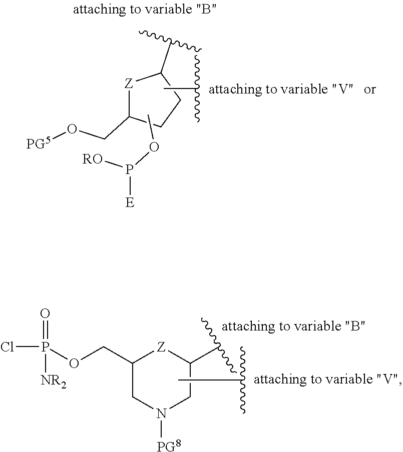

comprising the steps of:
(a) providing a compound of formula M2:

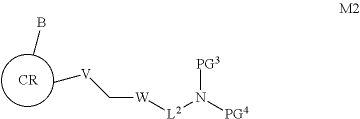

or a salt thereof, wherein

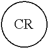

is

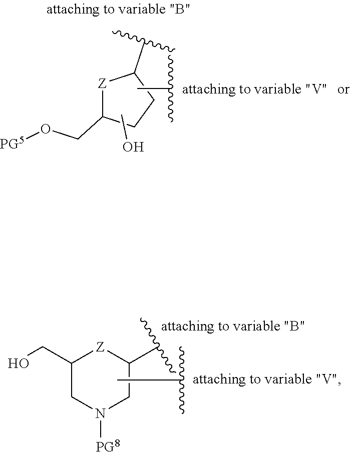

(b) reacting said compound of formula M2 with a P(III) or P(V) forming reagent to form a compound of formula P1, wherein:
PG³, PG⁴, and PG⁸ are independently hydrogen or a suitable nitrogen protecting group, provided both PG³ and PG⁴ are not hydrogen at the same time;
PG⁵ is hydrogen or a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

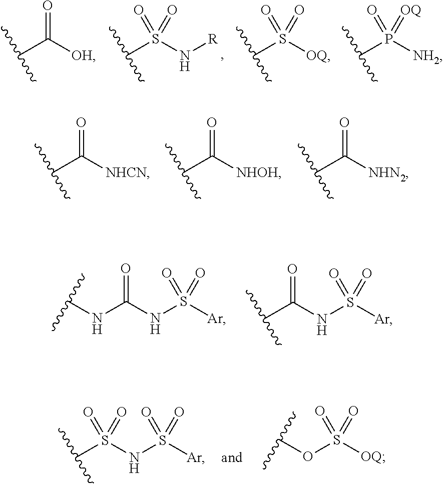

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the protecting group PG⁸ used for selective protection of a nitrogen group, for example, in nucleic acid or analogue thereof compound P1, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another aspect, the present invention provides a method for preparing a compound of formula P1-a:

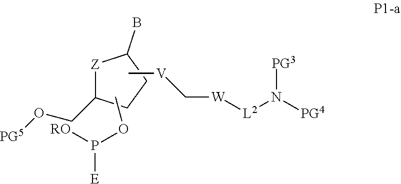

or a salt thereof, comprising the steps of:
(a) providing a compound of formula M2-a:

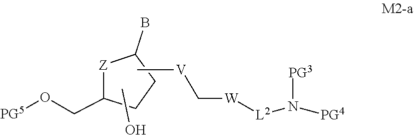

or a salt thereof, and
(b) reacting said compound of formula M2-a with a P(III) forming reagent to form a compound of formula P1-a, wherein:
PG³ and PG⁴ are independently hydrogen or a suitable nitrogen protecting group, provided both PG³ and PG⁴ are not hydrogen at the same time;
PG⁵ is hydrogen or a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

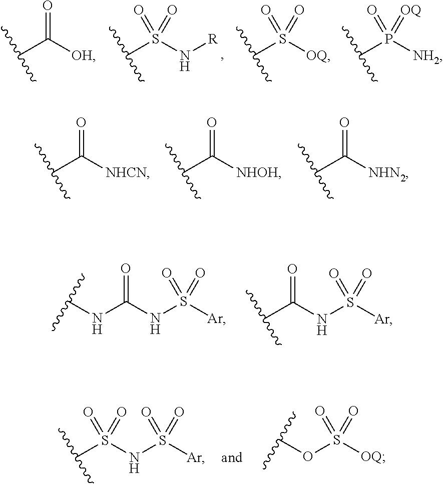

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to one embodiment, step (b) above is preformed using a P(III) forming reagent. In some embodiments, the P(III) forming reagent is 2-cyanoethyl phosphorodichloridite. One of ordinary skill would recognize that the displacement of a leaving group in a phosphoramidite forming reagent by the hydroxyl moiety of a compound of formula M2 or M2-a is achieved either with or without the presence of a suitable base. Such suitable bases are well known in the art and include organic and inorganic bases. In certain embodiments, the base is a tertiary amine such as triethylamine or diisopropylethylamine. In other embodiments, step (b) above is preformed using N,N-dimethylphosphoramic dichloride as a P(V) forming reagent.

According to another aspect, the present invention provides a method for preparing a nucleic acid or analogue thereof compound P2, or a pharmaceutically acceptable salt thereof, comprising

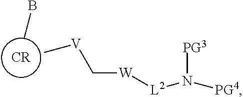

wherein

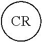

is

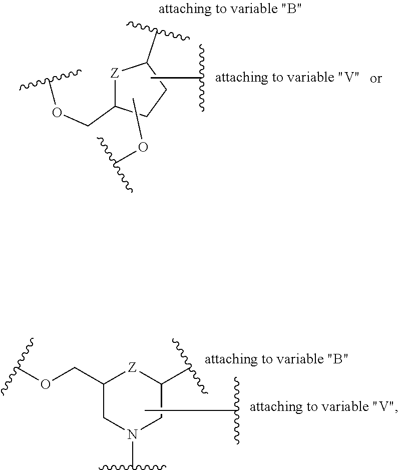

and
comprising the steps of:
(a) providing a compound of formula P1:

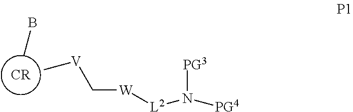

or a salt thereof, wherein

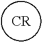

is

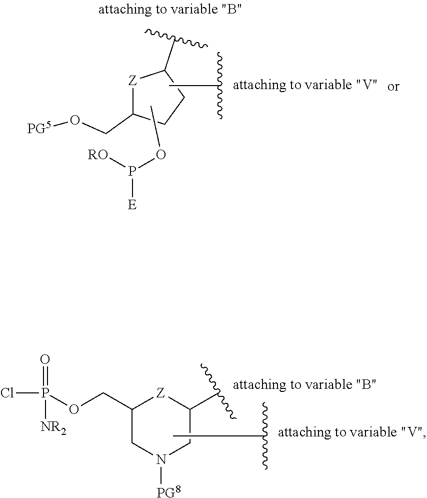

and
(b) synthesizing the nucleic acid or analogue thereof compound P2, or a pharmaceutically acceptable salt thereof, by solid phase synthesis incorporating one or more the compound of formula P1, or a salt thereof, wherein
PG³, PG⁴, and PG⁸ are independently hydrogen or a suitable nitrogen protecting group, provided both PG³ and PG⁴ are not hydrogen at the same time;
PG⁵ is hydrogen or a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

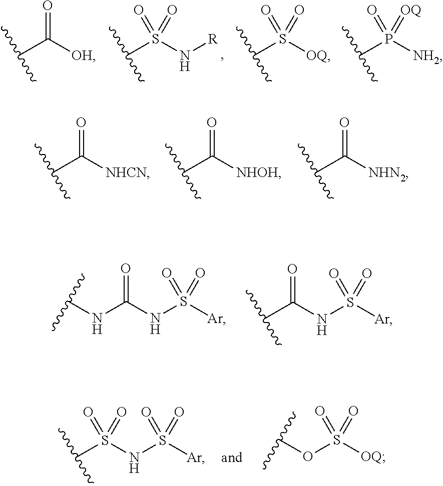

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to another aspect, the present invention provides a method for preparing a nucleic acid or analogue thereof compound P2-a, or a pharmaceutically acceptable salt thereof, comprising

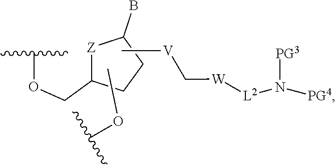

and comprising the steps of:
(a) providing a compound of formula P1-a:

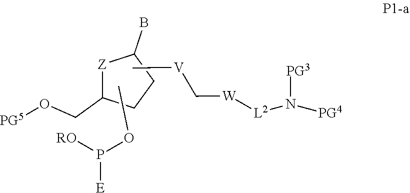

or a salt thereof, and
(b) synthesizing the nucleic acid or analogue thereof compound P2-a, or a pharmaceutically acceptable salt thereof, by solid phase synthesis incorporating one or more the compound of formula P1-a, or a salt thereof, wherein
PG³ and PG⁴ are independently hydrogen or a suitable nitrogen protecting group, provided both PG³ and PG⁴ are not hydrogen at the same time;
PG⁵ is hydrogen or a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

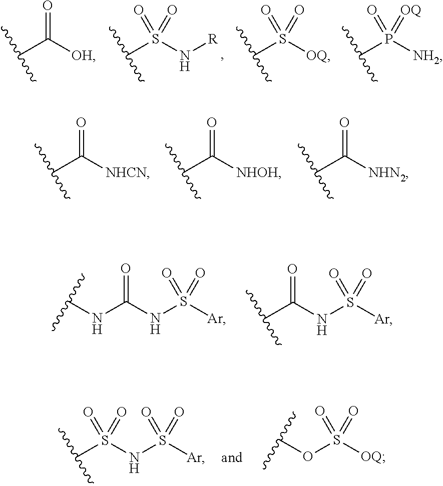

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to one embodiment, the nucleic acid or analogue thereof forming conditions in step (b) above is preformed using known and commonly applied processes to prepare nucleic acids or analogues thereof in the art. For example, the compound of formula P1 or P1-a, or a salt thereof, is coupled to a solid supported nucleic acid or analogue thereof bearing a 5′-hydoxyl group. Further steps can comprise one or more deprotections, couplings, phosphite oxidatation, and/or cleavage from the solid support to provide nucleic acids or analogues thereof of various nucleotide lengths including a nucleic acid or analogue thereof compound P2 or P2-a, or a pharmaceutically acceptable salt thereof.

According to alternate aspect, the present invention provides a method for preparing a nucleic acid or analogue thereof compound P3, or a pharmaceutically acceptable salt thereof, comprising

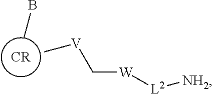

and comprising the steps of:
(a) providing a nucleic acid or analogue thereof compound P2, or a pharmaceutically acceptable salt thereof, comprising

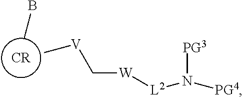

and
(b) deprotecting said nucleic acid or analogue thereof compound P2, or a pharmaceutically acceptable salt thereof, to form the nucleic acid or analogue thereof compound P3, or a pharmaceutically acceptable salt thereof, wherein:

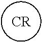

is

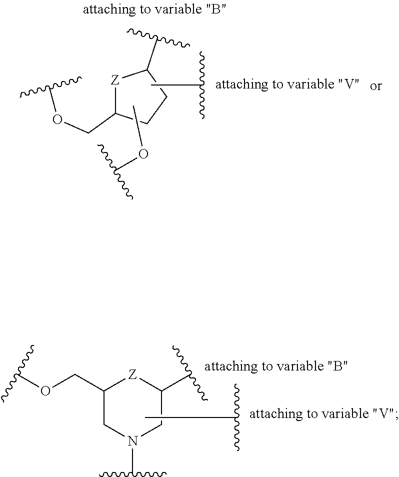

PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

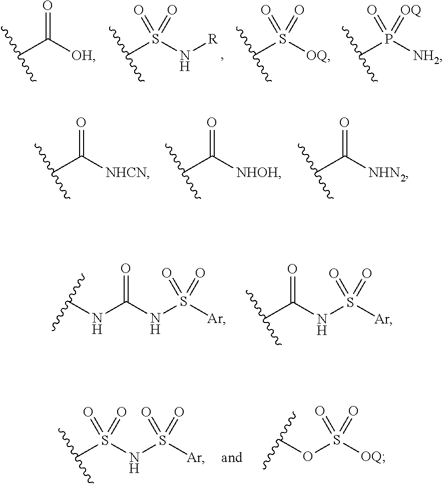

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

According to alternate aspect, the present invention provides a method for preparing a nucleic acid or analogue thereof compound P3-a, or a pharmaceutically acceptable salt thereof, comprising

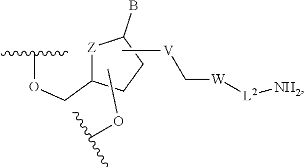

and comprising the steps of:
(a) providing a nucleic acid or analogue thereof compound P2-a, or a pharmaceutically acceptable salt thereof, comprising

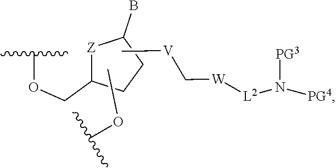

and
(b) deprotecting said nucleic acid or analogue thereof compound P2-a, or a pharmaceutically acceptable salt thereof, to form the nucleic acid or analogue thereof compound P3-a, or a pharmaceutically acceptable salt thereof, wherein:
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

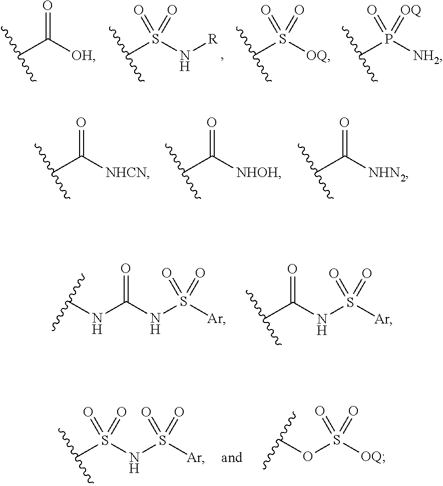

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

The PG³ and PG⁴ groups of the nucleic acid or analogue thereof compound P2 or P2-a, or a pharmaceutically acceptable salt thereof, are each independently hydrogen or a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of PG³ and PG⁴ groups of the nucleic acid or analogue thereof compound P2 or P2-a, or a pharmaceutically acceptable salt thereof, include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. In other embodiments, the PG³ and PG⁴ groups of the nucleic acid or analogue thereof compound P2 or P2-a, or a pharmaceutically acceptable salt thereof, are taken together with their intervening nitrogen atom to form a heterocyclic protecting group, such as a pyrrole or pyrrolidine-2,5-dione.

Removal of protecting groups (e.g., both PG³ and PG⁴ or either of PG³ or PG⁴ independently) of the nucleic acid or analogue thereof compound P2 or P2-a, or a pharmaceutically acceptable salt thereof, affords nucleic acid or analogue thereof compound P3 or P2-a or pharmaceutically acceptable salt thereof. In some embodiments, PG³ or PG⁴ comprise carbamate derivatives that can be removed under acidic or basic conditions. In certain embodiments, the protecting groups (e.g., both PG³ and PG⁴ or either of PG³ or PG⁴ independently) of the nucleic acid or analogue thereof compound P2 or P2-a, or a pharmaceutically acceptable salt thereof, are removed by acid hydrolysis. It will be appreciated that upon acid hydrolysis of the protecting groups of the nucleic acid or analogue thereof compound P2 or P2-a, a salt of the nucleic acid or analogue thereof compound P3 or P3-a may be formed. One of ordinary skill in the art would recognize that a wide variety of acids are useful for removing amino protecting groups that are acid-labile and therefore a wide variety of salt forms of a nucleic acid or analogue thereof compound P3 or P3-a are contemplated.

In other embodiments, the protecting groups (e.g., both PG³ and PG⁴ or either of PG³ or PG⁴ independently) of nucleic acid or analogue thereof compound P2 or P2-a, or a pharmaceutically acceptable salt thereof, are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-Diazabicyclo[5.4.0]undec-7-ene (DBU).

According to another alternative aspect, the present invention provides a method for preparing a nucleic acid or analogue thereof compound P4, or a pharmaceutically acceptable salt thereof, comprising

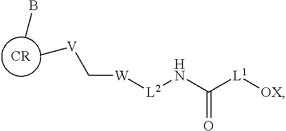

and comprising the steps of:
(a) providing a compound of formula F-3:

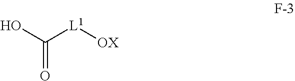

or a pharmaceutically acceptable salt thereof, and
(b) reacting said fragment compound of formula F-3 with a nucleic acid or analogue thereof compound P3, or a pharmaceutically acceptable salt thereof, comprising

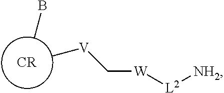

to provide the compound of formula P4, or a pharmaceutically acceptable salt thereof, wherein:

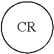

is

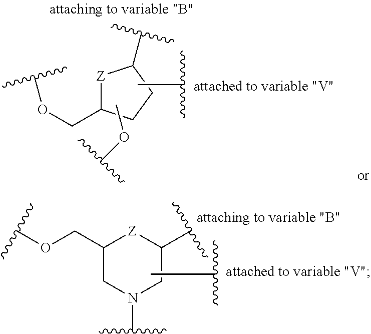

B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

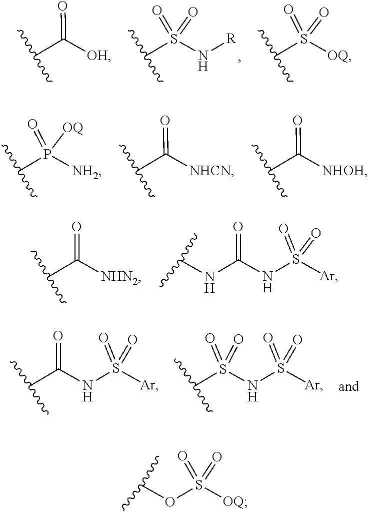

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

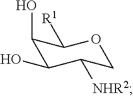

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to another alternative aspect, the present invention provides a method for preparing a nucleic acid or analogue thereof compound P4-a, or a pharmaceutically acceptable salt thereof, comprising

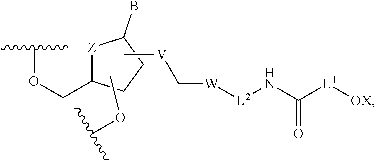

and comprising the steps of:
(a) providing a compound of formula F-3:

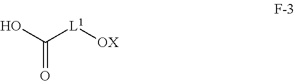

or a pharmaceutically acceptable salt thereof, and
(b) reacting said fragment compound of formula F-3 with a nucleic acid or analogue thereof compound P3-a, or a pharmaceutically acceptable salt thereof, comprising

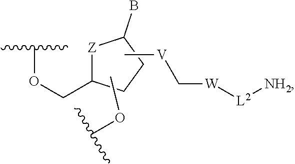

to provide the compound of formula P4-a, or a pharmaceutically acceptable salt thereof, wherein:
B is a nucleobase or hydrogen;
   each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

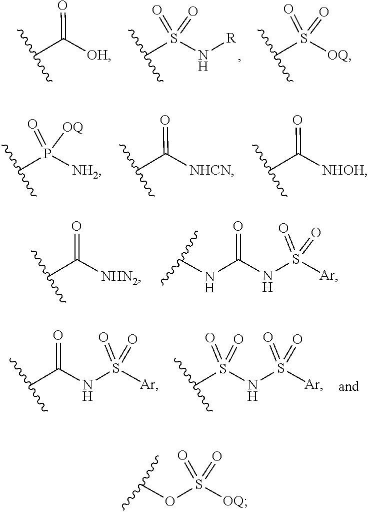

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

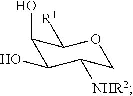

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to one embodiment, the amidation reaction of step (b) can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the carboxylic acid of the fragment compound of formula F-3 is converted to an activated ester, followed by reacting with an amine compound. In certain embodiments, the activated ester forming conditions include a mixture of NHS (N-hydroxysuccinimide and EDC [1-ethyl-3-(3-dimethylaminopropyl)carbodiimide].

Without being limited to the current disclosure, the assembly of fragment compound of formula F-3 with the nucleic acid or analogue thereof compound P3 or P3-a in step (b) above could be facilitated using a range of cross-linking technologies. It is within the purview of those having ordinary skill in the art that the carboxylic acid of the fragment compound of formula F-3 and the amine of the nucleic acid or analogue thereof compound P3 or P3-a could be replaced by suitable coupling moieties that react with each other to covalently link the fragment compound of formula F-3 with the nucleic acid or analogue thereof compound P3 or P3-a by alternative means. Exemplary cross-linking technologies envisioned for use in the current disclosure also include those listed in Table 1 disclosed herein.

Accordingly, in certain embodiments, the present invention provides a compound of formula

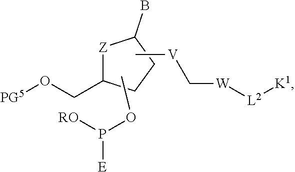

or a nucleic acid or analogue thereof compound comprising

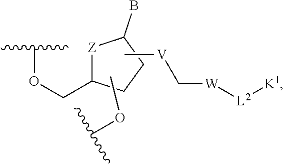

or a pharmaceutically acceptable salt thereof, wherein each of PG⁵, B, E, L², V, W, R, and Z is as defined and in classes and subclasses as described herein, and each of K¹ and K² is independently selected from the coupling moieties listed in Table 1. In some embodiments, the present invention provides a nucleic acid or analogue thereof compound comprising

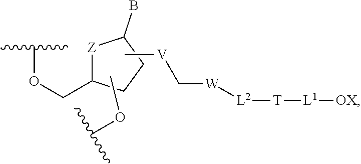

or a pharmaceutically acceptable salt thereof, wherein each of B, X, L¹, L², V, W, and Z is as defined and in classes and subclasses as described herein, and T is selected from the linkers listed in Table 1.

According to another alternative aspect, the present invention provides a method for preparing a fragment compound of formula F-7:

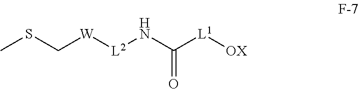

or a salt thereof, comprising the steps of:
(a) providing a fragment compound of formula F-6:

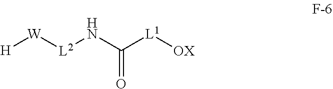

or a salt thereof, and
(b) alkylating said fragment compound of formula F-6 to form the fragment compound of formula F-7, wherein:
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

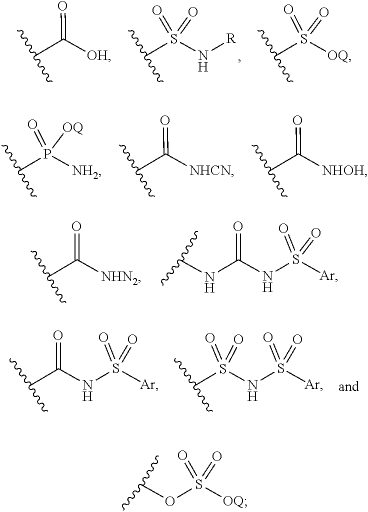

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
X is a ligand selected from GalNAc, D-mannose, L-galactose, D-arabinose, L-fucose, polyols, and

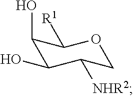

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
W is —O—, —S—, or —NR—.

According to some aspects, the alkylation at step (b) above is achieved by reacting a fragment compound of formula F-6 with a mixture of DMSO and acetic anhydride under acidic conditions. In certain embodiments, when W—H is a hydroxyl group, the mixture of DMSO and acetic anhydride in the presence of acetic acid forms (methylthio)methyl acetate in situ via the Pummerer rearrangement which then reacts with the hydroxyl group of the fragment compound of formula F-6 to provide a monothioacetal functionalized fragment compound of formula F-7. In certain embodiments, the alkylation is achieved using an organic acid, such as acidic acid at an elevated temperature, e.g., about 30° C. to about 70° C.

According to another alternative aspect, the present invention provides a method for preparing a compound of formula D′:

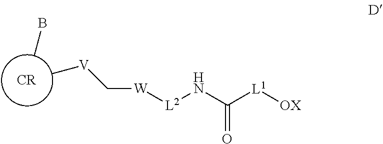

or a salt thereof, comprising the steps of:
(a) providing a compound of formula F-7:

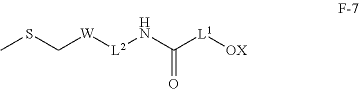

or a salt thereof, and

(b) reacting said fragment compound of formula F-7 with a compound of formula I′:

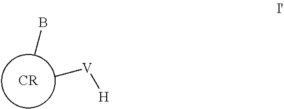

or a salt thereof, to provide the compound of formula D′, wherein:

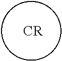

is

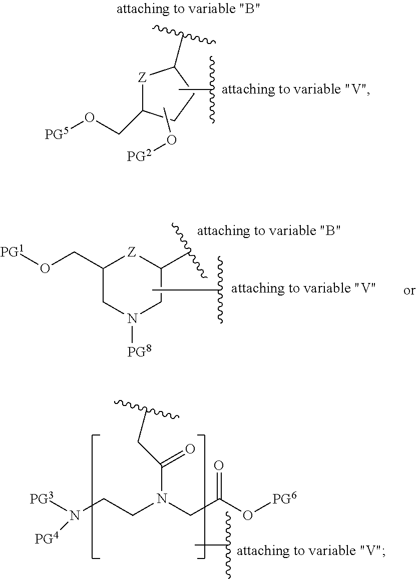

PG¹, PG², and PG are independently hydrogen or a suitable hydroxyl protecting group;
PG³, PG⁴, and PG⁸ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

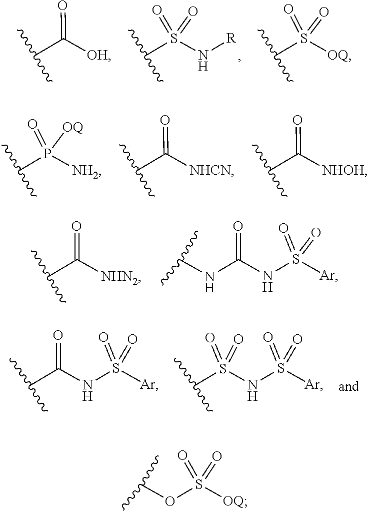

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

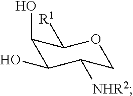

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, the protecting group PG⁸ used for selective protection of a nitrogen group, for example, in formulas D′ and I′, includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

According to another alternative aspect, the present invention provides a method for preparing a compound of formula D′-a:

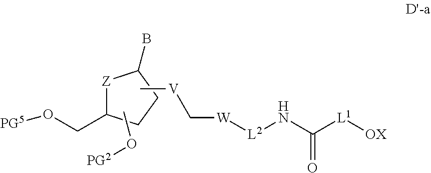

or a salt thereof, comprising the steps of:
(a) providing a compound of formula F-7:

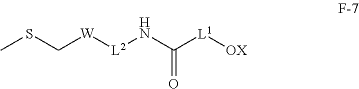

or a salt thereof, and
(b) reacting said fragment compound of formula F-7 with a compound of formula I′:

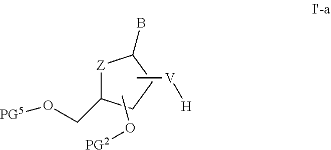

or a salt thereof,
to provide the compound of formula D′-a, wherein:
PG⁵ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

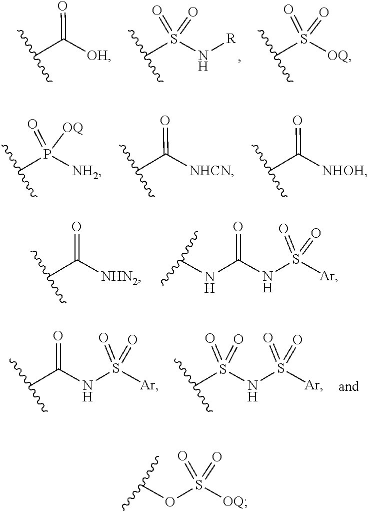

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

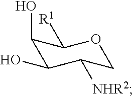

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to one embodiment, step (b) above is performed under mild oxidizing and/or acidic conditions. In some embodiments, V is —O—. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

According to another alternative aspect, the present invention provides a method for preparing a compound of formula B:

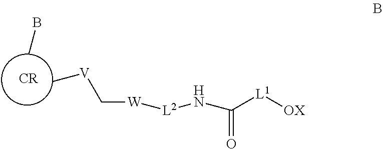

or a salt thereof, wherein

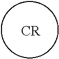

is

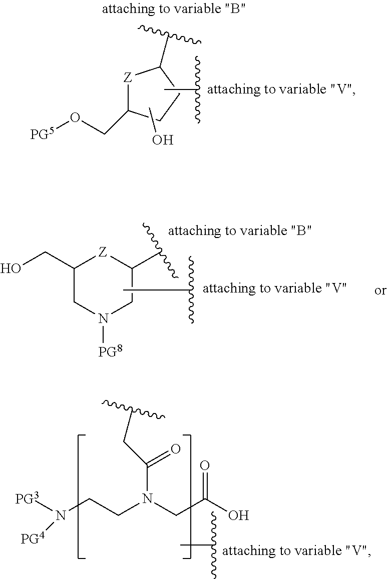

comprising the steps of:
(a) providing a compound of formula D′:

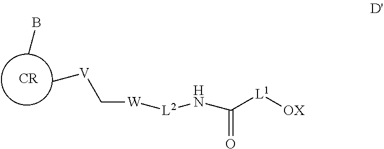

or a salt thereof, wherein

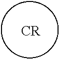

is

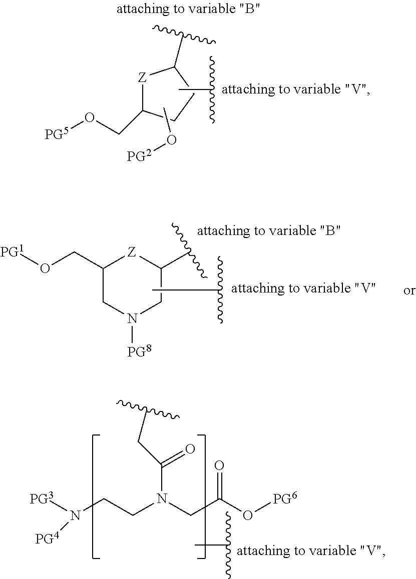

and
(b) deprotecting a compound of formula D′, to provide the compound of formula B, wherein:
PG¹, PG², and PG⁵ are independently hydrogen or a suitable hydroxyl protecting group;
PG³, PG⁴, and PG⁸ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

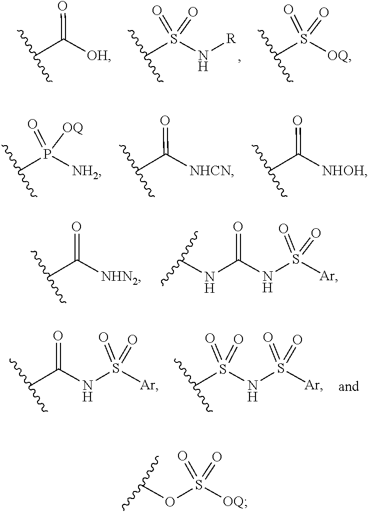

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

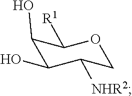

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to another alternative aspect, the present invention provides a method for preparing a compound of formula B-a:

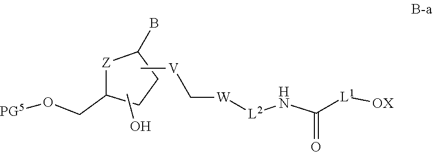

or a salt thereof, comprising the steps of:
(a) providing a compound of formula D′-a:

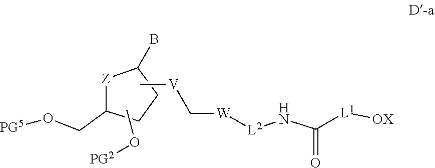

or a salt thereof, and
(b) deprotecting a compound of formula D′-a,
to provide the compound of formula B-a,
wherein:
each PG⁵ and PG² are independently hydrogen or a suitable hydroxyl protecting group; B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

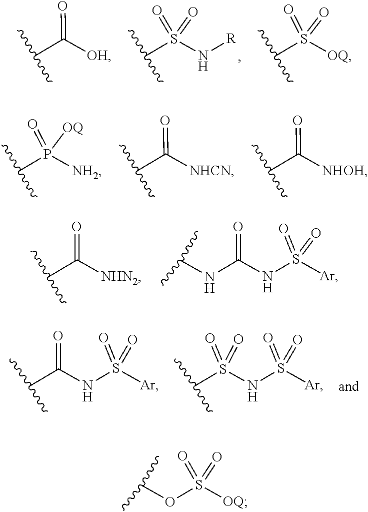

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

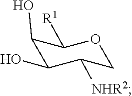

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

According to one embodiment, PG² and PG³ removed in step (b) above is selected from suitable hydroxyl or nitrogen protecting groups. Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, each of PG¹ and PG², taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Suitable amino protecting groups are well known in the art and include those described in detail in Protecting Groups in Organic Synthesis, T. W. Greene and P. G. M. Wuts, 3P edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. Examples of the PG³ group deprotected in step (b) above include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

In some embodiments, the present invention provides a compound which is selected from the starting materials, intermediates, and products, as described in the methods, or salts thereof.

7. Compounds of the Invention

In certain embodiments, the present invention provides a compound of formula A:

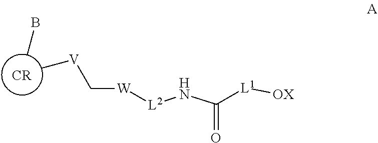

or a pharmaceutically acceptable salt thereof, wherein:

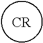

is

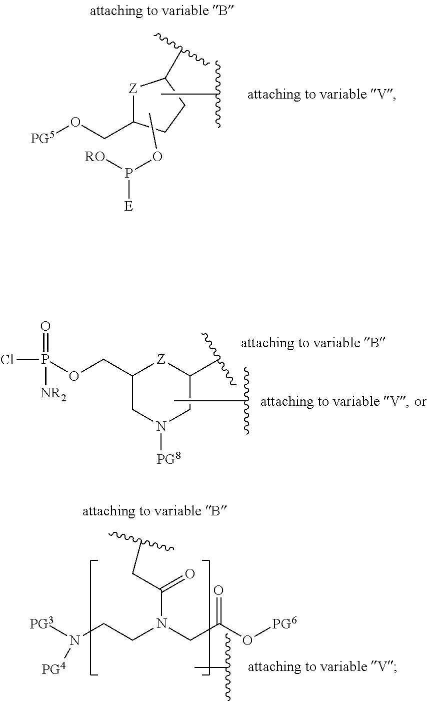

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
PG⁶ is hydrogen or a suitable carboxylate protecting group;
B is a nucleobase or hydrogen;
E is halogen or NR₂;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

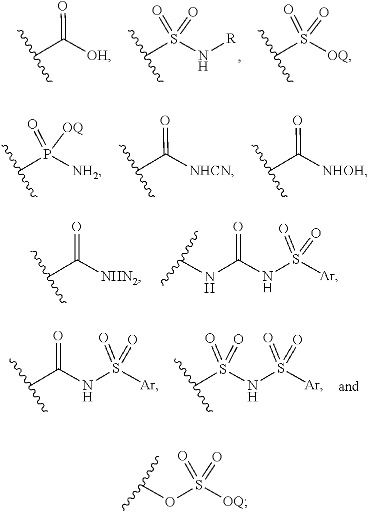

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

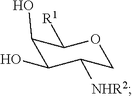

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,

Suitable carboxylate protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable carboxylate protecting groups include, but are not limited to, substituted C₁₋₆ aliphatic esters, optionally substituted aryl esters, silyl esters, activated esters (e.g., derivatives of nitrophenol, pentafluorophenol, N-hydroxylsuccinimide, hydroxybenzotriazole, etc.), orthoesters, and the like. Examples of such ester groups include methyl, ethyl, propyl, isopropyl, butyl, isobutyl, tert-butyl, benzyl, and phenyl wherein each group is optionally substituted.

In certain embodiments, the present invention provides a compound of formula A-a:

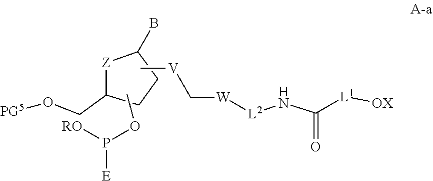

or a pharmaceutically acceptable salt thereof, wherein:
PG⁵ is a suitable hydroxyl protecting group;
B is a nucleobase or hydrogen;
E is a halogen or NR₂;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

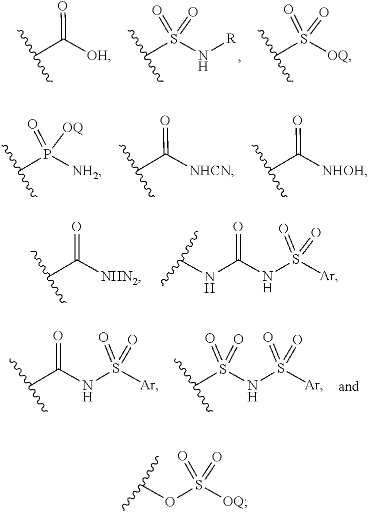

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

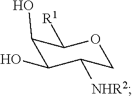

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, B of a compound of formula A or A-a is hydrogen. In certain embodiments, B of a compound of formula A or A-a is guanine (G), cytosine (C), adenine (A), thymine (T), or uracil (U), or derivatives thereof, such as protected derivatives suitable for use in the preparation of oligionucleotides. In some embodiments, each of nucleobases G, A, and C independently comprises a protecting group selected from isobutyryl, phenoxyacetyl, isopropylphenoxyacetyl, benzoyl, and acetyl.

In certain embodiments, a compound of formula A or A-a is not

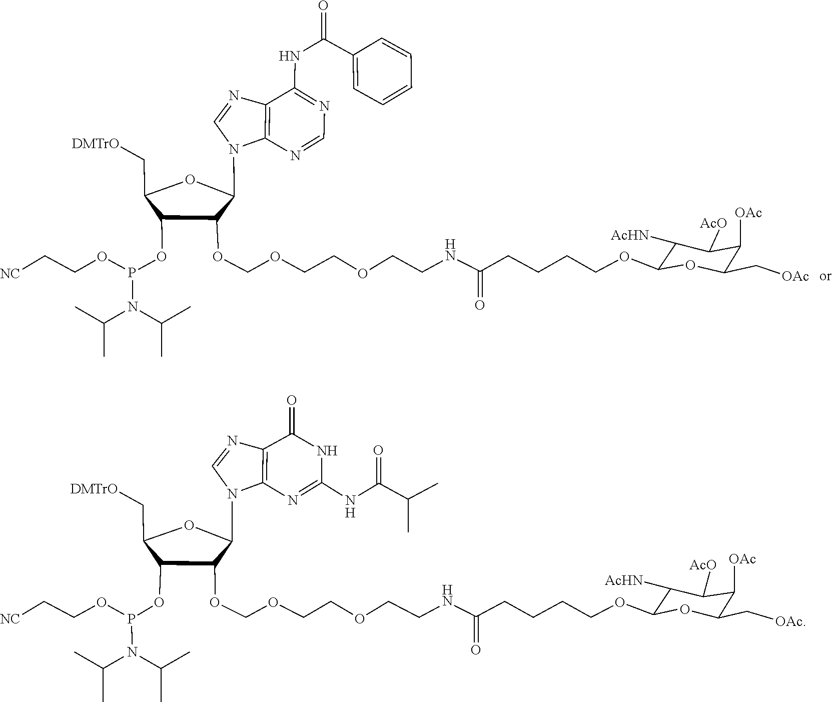

In certain embodiments, the present invention provides a compound of formula A1:

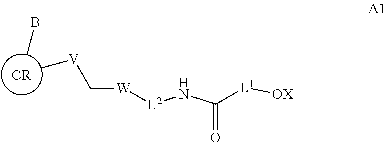

or a pharmaceutically acceptable salt thereof, wherein:

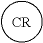

is

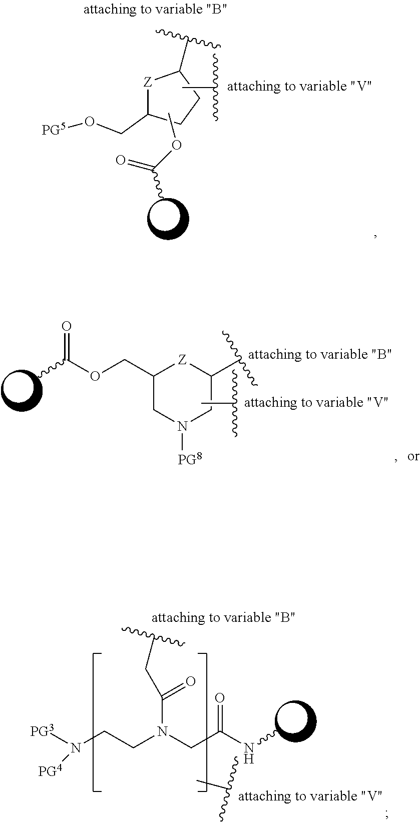

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

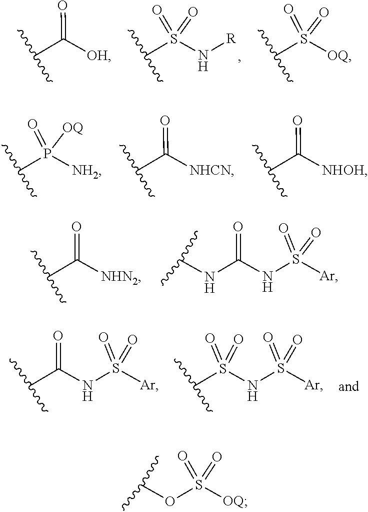

each R is independently selected from hydrogen, alkyl, alkenyl, aromatic, heterocycle, substituted alkyl, and substituted alkenyl;
Q is H or a salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY; V and W are independently —O—, —S—, or —NR—;
X is a ligand selected from GalNAc, D-mannose, L-galactose, D-arabinose, L-fucose, polyols, and

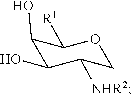

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, the present invention provides a compound of formula A1:

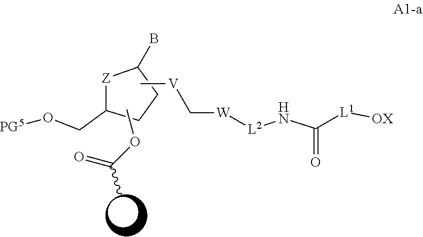

or a pharmaceutically acceptable salt thereof, wherein:
PG⁵ is a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

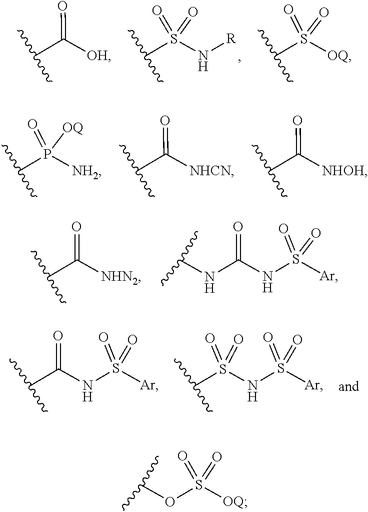

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

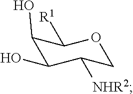

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, the present invention provides a compound of formula B:

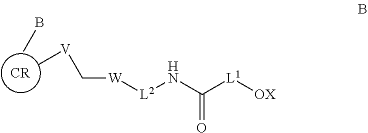

or a pharmaceutically acceptable salt thereof, wherein:

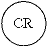

is

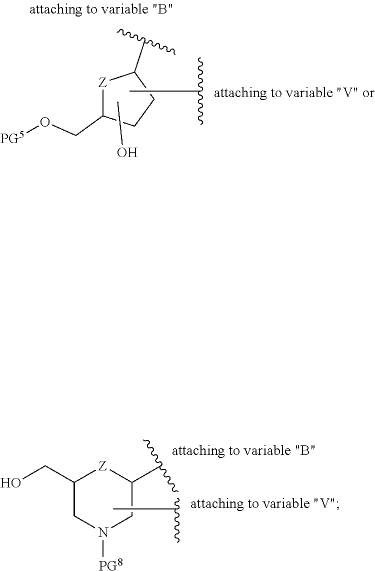

PG⁵ is hydrogen or a suitable hydroxyl protecting group;
PG⁸ is hydrogen or a suitable nitrogen protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

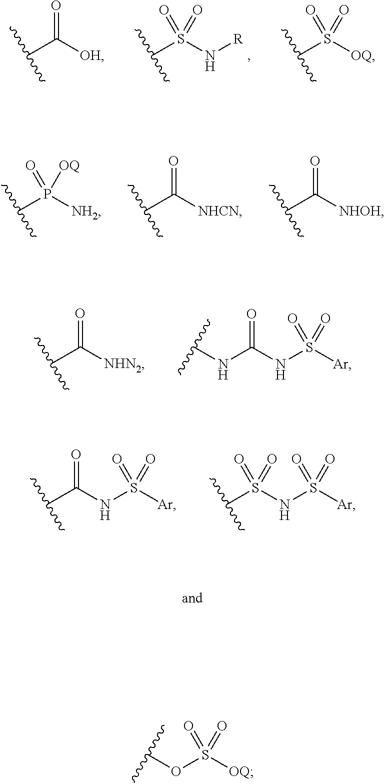

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

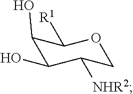

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, the present invention provides a compound of formula B-a:

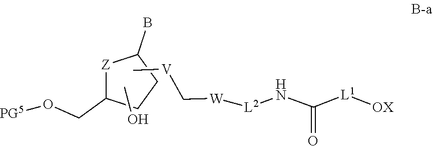

or a pharmaceutically acceptable salt thereof, wherein:
PG⁵ is a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

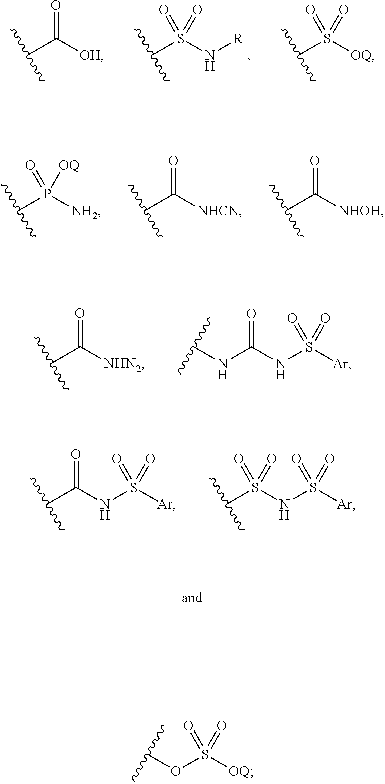

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

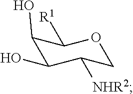

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, a compound of formula B or B-a is not

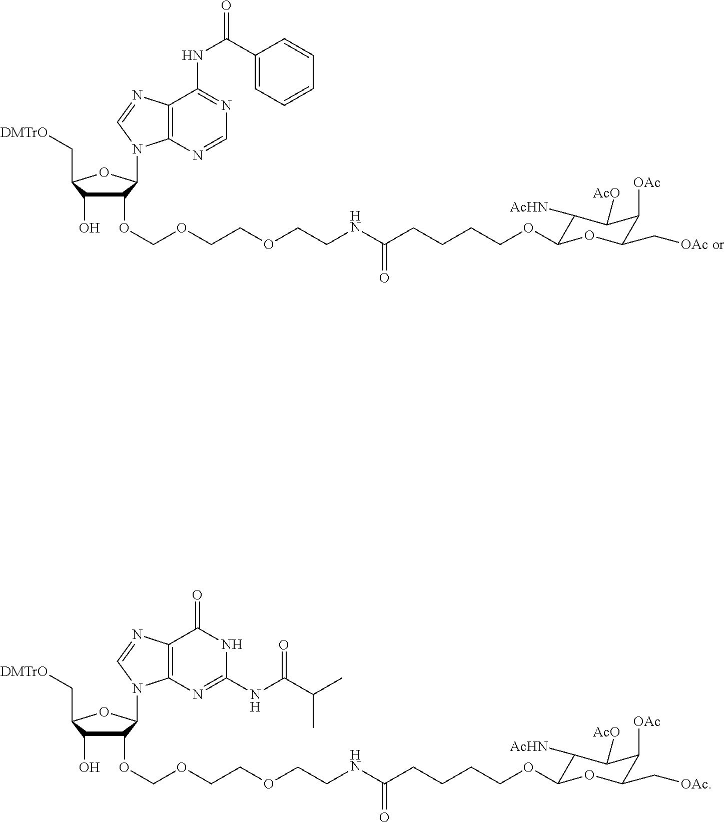

In certain embodiments, the present invention provides a compound of formula C-a:

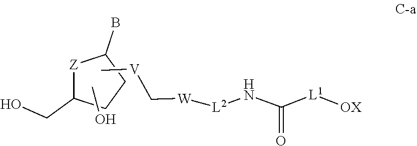

or a pharmaceutically acceptable salt thereof, wherein:
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

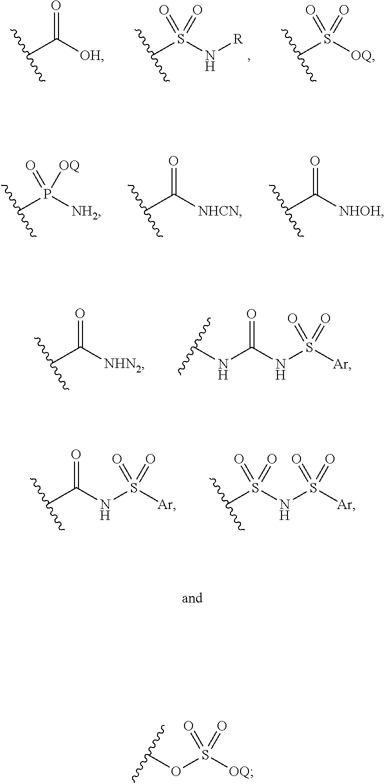

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

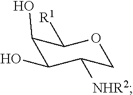

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, a compound of formula C is not

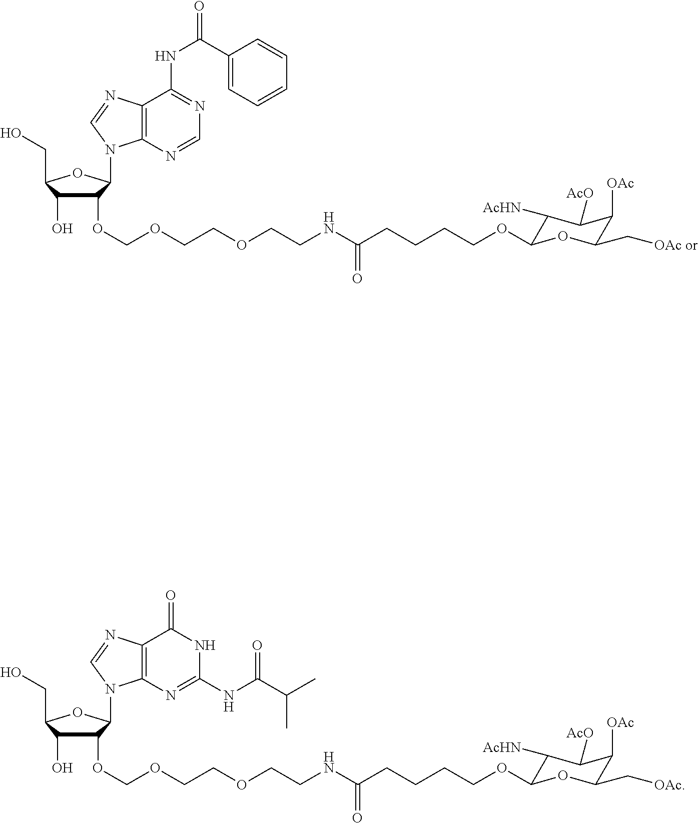

In certain embodiments, the present invention provides a compound of formula D-a:

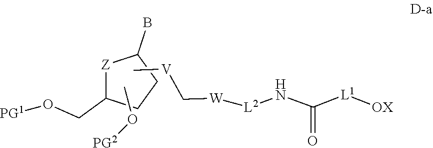

or a pharmaceutically acceptable salt thereof, wherein:
PG¹ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

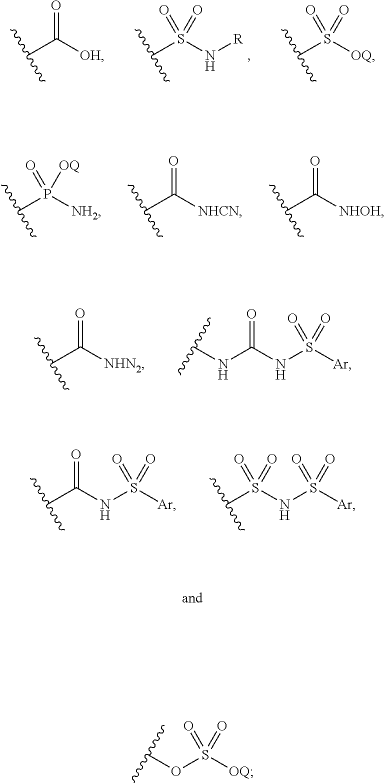

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

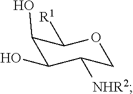

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, a compound of formula D is not

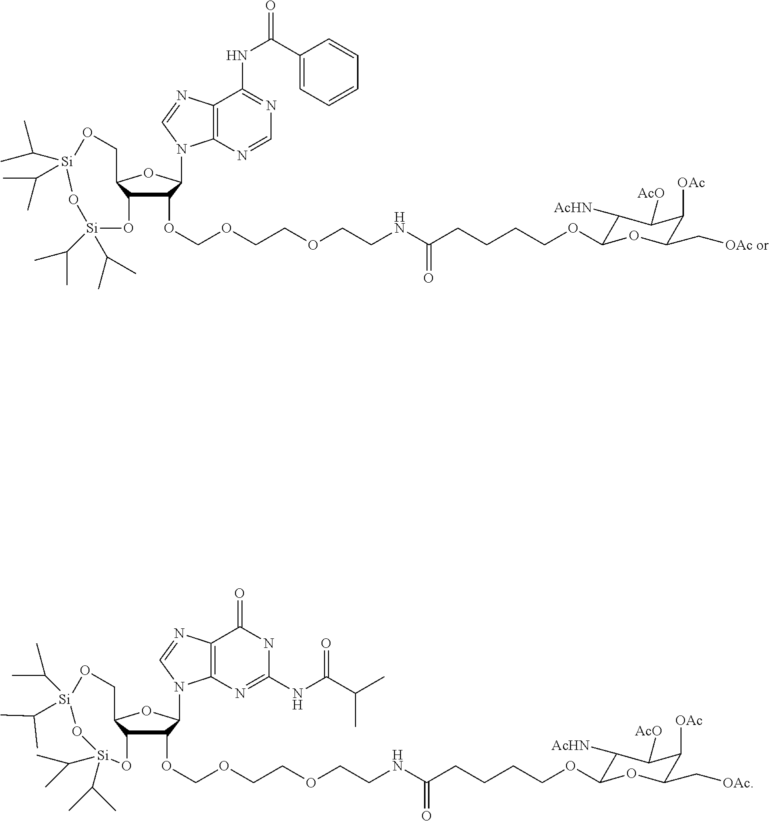

In certain embodiments, the present invention provides a compound of formula F-6:

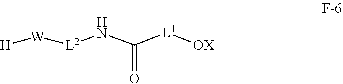

or a pharmaceutically acceptable salt thereof, wherein:
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

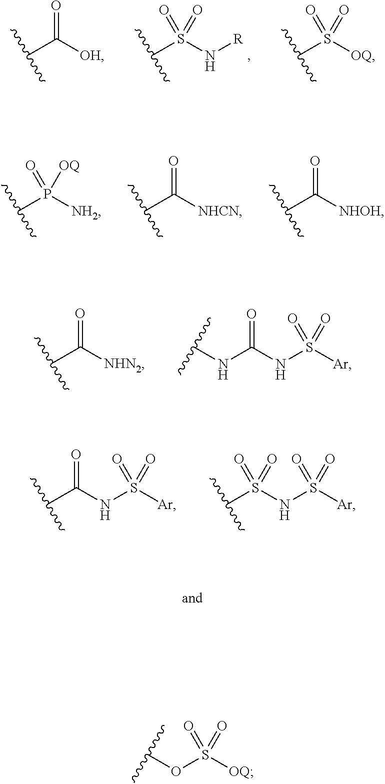

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
X is a ligand selected from GalNAc, D-mannose, L-galactose, D-arabinose, L-fucose, polyols, and

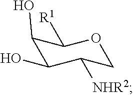

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
W is —O—, —S—, or —NR—.

In certain embodiments, the present invention provides a compound of formula F-5:

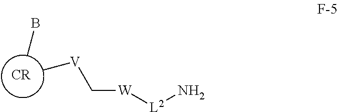

or a salt thereof, wherein:

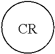

is

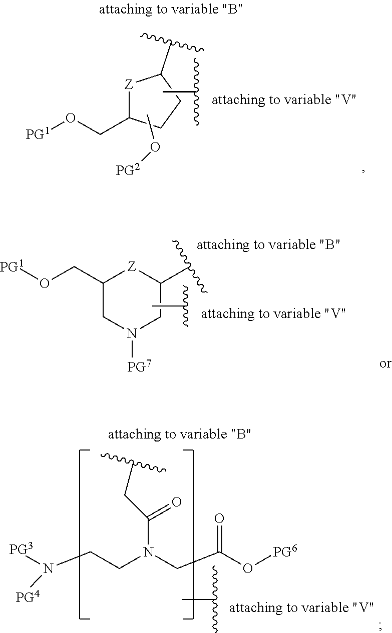

PG¹ and PG² are independently hydrogen or a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

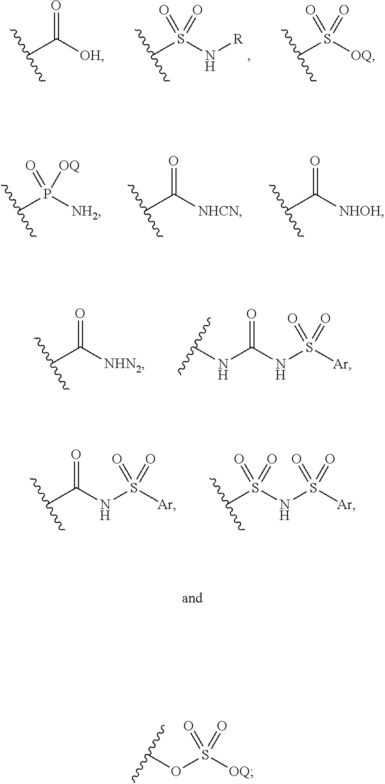

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula F-5-a:

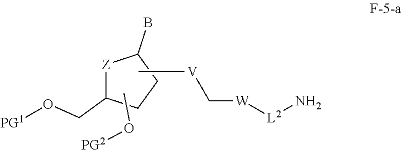

or a salt thereof, wherein:
PG¹ and PG² are independently a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

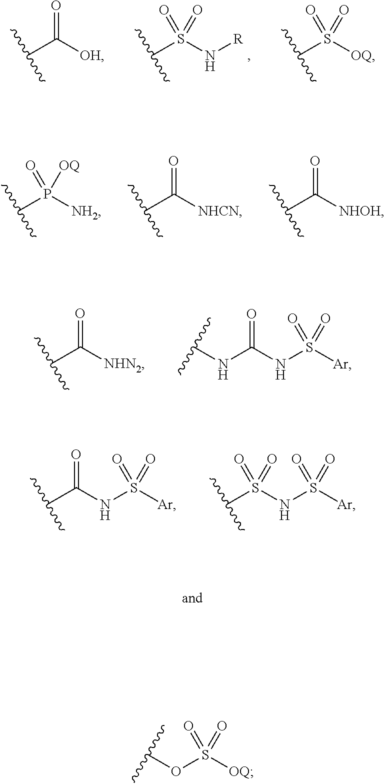

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, a compound of formula F-5 is not

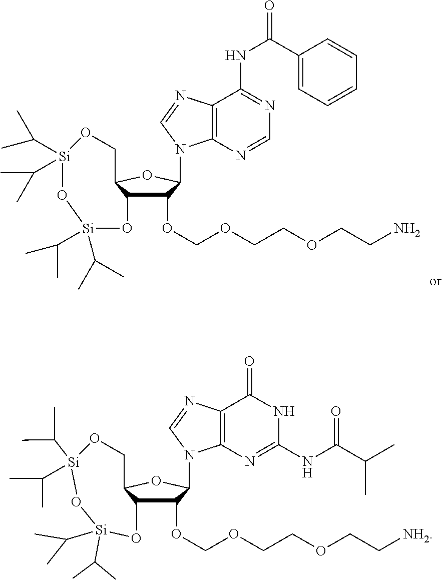

In some embodiments, the present invention provides a salt of a compound of formula F-5 or F-5-a. In some embodiments, the present invention provides a fumaric acid salt of a compound of formula F-5 or F-5-a. In some embodiments, the present invention provides a bifumarate salt of a compound of formula F-5 or F-5-a. In some embodiments, a fumaric acid salt of a compound of formula F-5 or F-5-a is in crystal form. In certain embodiments, the present invention provides a bifumarate salt of a compound of formula F-5 or F-5-a, the bifumarate salt being crystalline and having reduced solidification in comparison to other salt forms.

In certain embodiments, the present invention provides a compound of formula F-4:

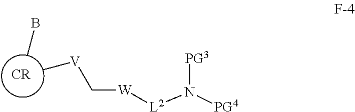

or a pharmaceutically acceptable salt thereof, wherein:

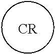

is

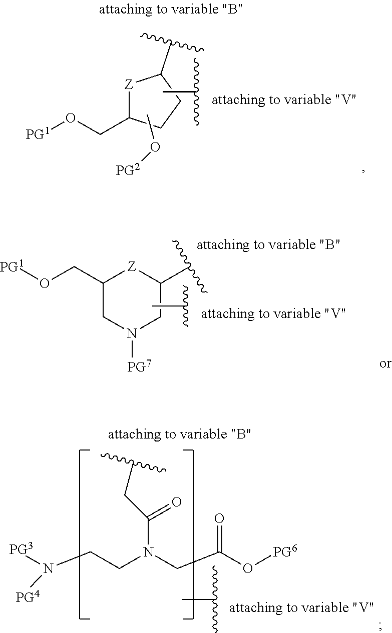

PG¹ and PG² are independently hydrogen or a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

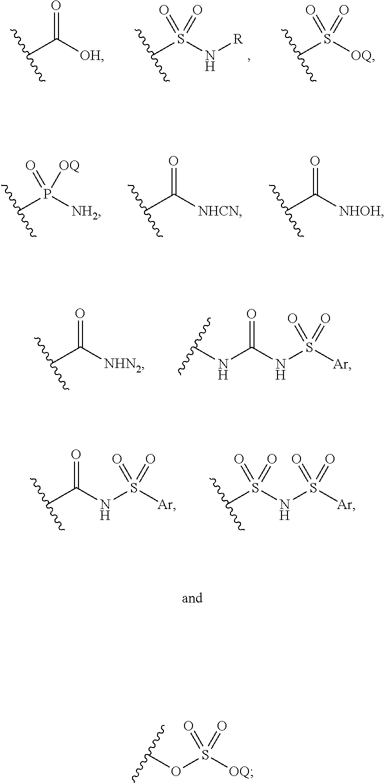

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula F-4-a:

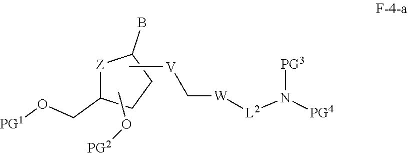

or a pharmaceutically acceptable salt thereof, wherein:
PG¹ and PG² are independently a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

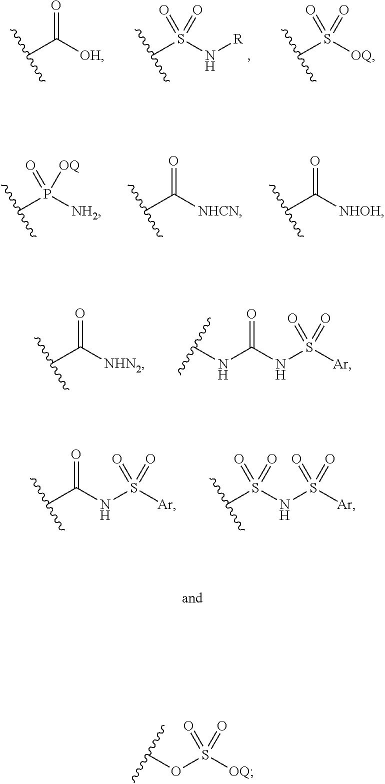

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, a compound of formula F-4 is not:

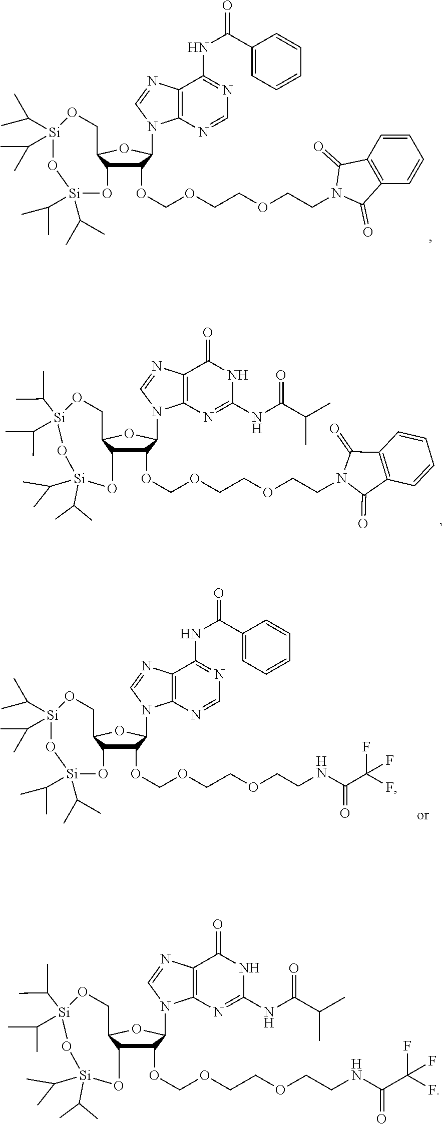

In certain embodiments, the present invention provides a compound of formula F-1:

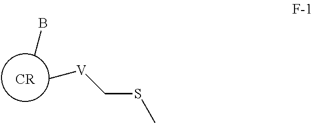

or a pharmaceutically acceptable salt thereof, wherein:

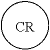

is

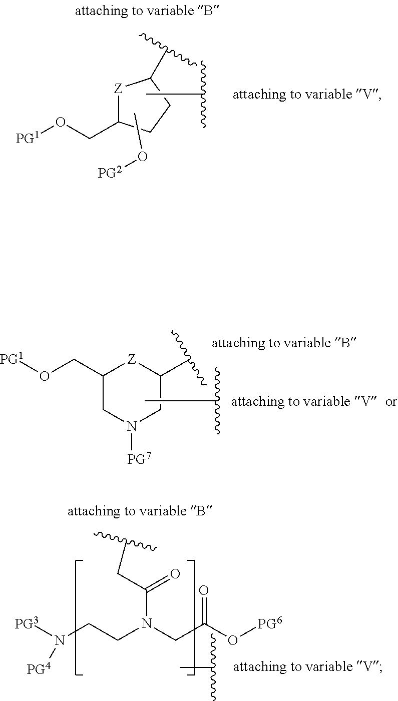

PG¹ and PG² are independently hydrogen or a suitable hydroxyl protecting group;
PG³, PG⁴, and PG⁷ are independently hydrogen or a suitable nitrogen protecting group, provided both PG³ and PG⁴ on the same nitrogen are not hydrogen at the same time;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula F-1-a:

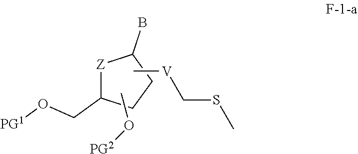

or a pharmaceutically acceptable salt thereof, wherein:
PG¹ and PG² are independently a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, a compound of formula F-1 is not:

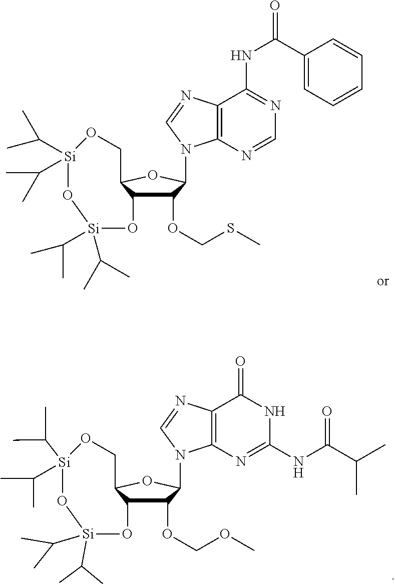

In certain embodiments, the present invention provides a compound of formula N1:

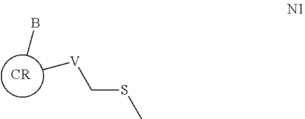

or a pharmaceutically acceptable salt thereof, wherein:

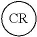

is

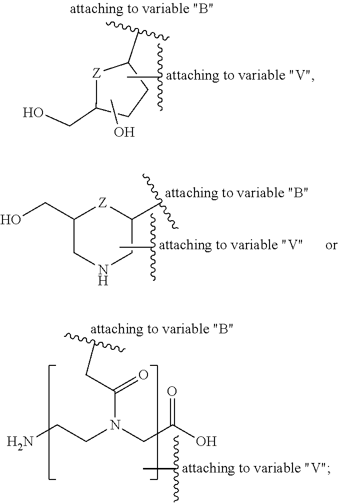

B is a nucleobase or hydrogen;
V and W are independently —O—, —S—, or —NR—;
each R is independently selected from hydrogen, alkyl, alkenyl, aromatic, heterocycle, substituted alkyl, substituted alkenyl;
Z is —CH₂—, —O—, —S—, or —NR—.

In certain embodiments, the present invention provides a compound of formula N1-a:

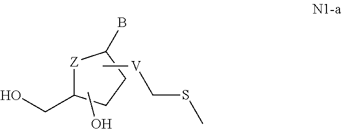

or a pharmaceutically acceptable salt thereof, wherein:
B is a nucleobase or hydrogen;
V and W are independently —O—, —S—, or —NR—;
each R is independently selected from hydrogen, alkyl, alkenyl, aromatic, heterocycle, substituted alkyl, substituted alkenyl;
Z is —CH₂—, —O—, —S—, or —NR—.

In certain embodiments, the present invention provides a compound of formula N2:

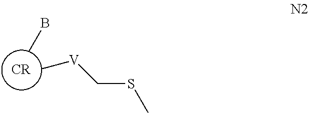

or a pharmaceutically acceptable salt thereof, wherein:

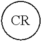

is

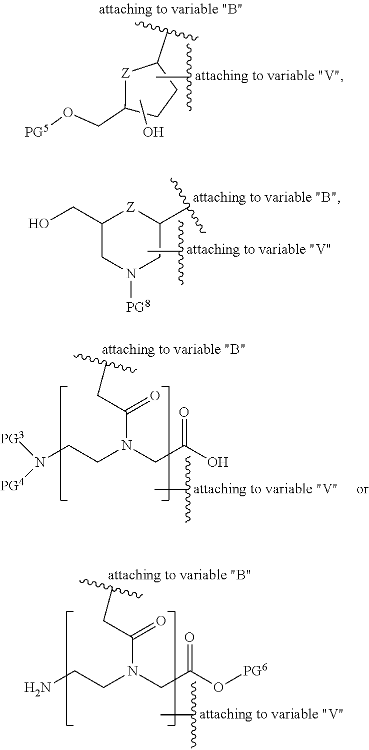

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
PG⁶ is hydrogen or a suitable carboxylate protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula N2-a:

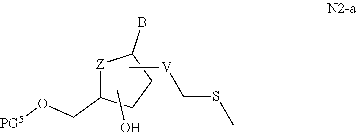

or a pharmaceutically acceptable salt thereof, wherein:
PG⁵ is hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula N3:

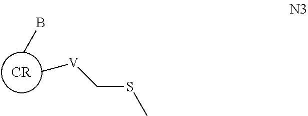

or a pharmaceutically acceptable salt thereof, wherein:

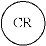

is

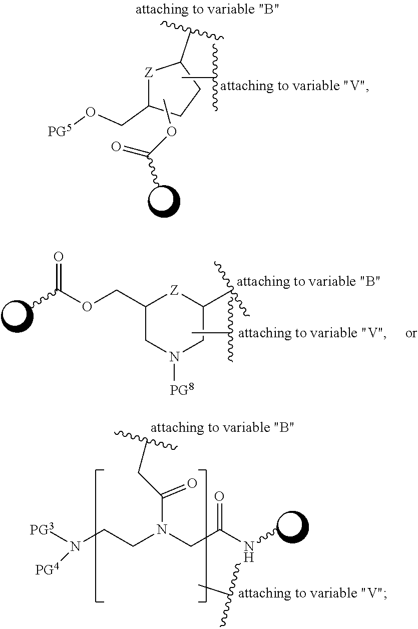

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula N3-a:

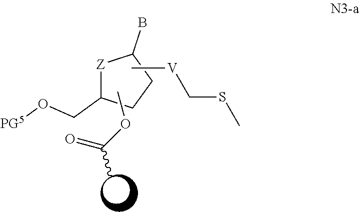

or a pharmaceutically acceptable salt thereof, wherein:
PG⁵ is hydrogen or a suitable hydroxyl protecting group;
B is a nucleobase or hydrogen;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula M1:

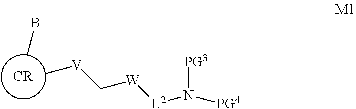

or a pharmaceutically acceptable salt thereof, wherein:

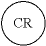

is

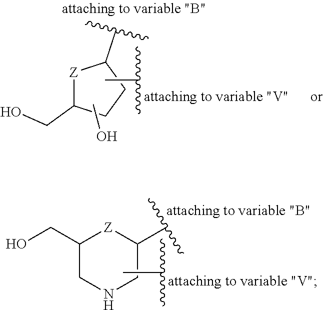

B is a nucleobase or hydrogen;
PG³ and PG⁴ are independently hydrogen or a suitable nitrogen protecting group, provided both PG³ and PG⁴ are not hydrogen at the same time;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

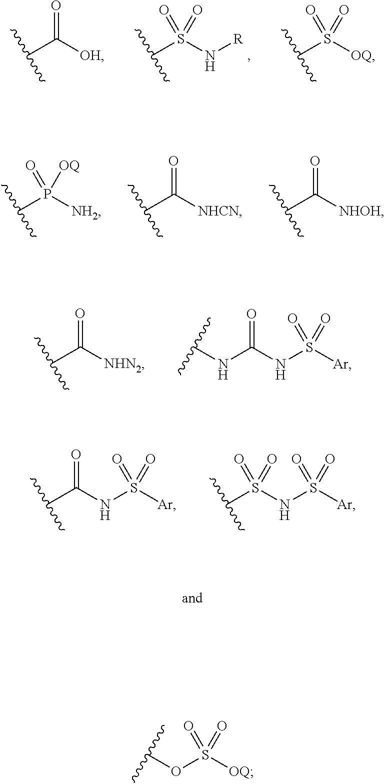

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula M1-a:

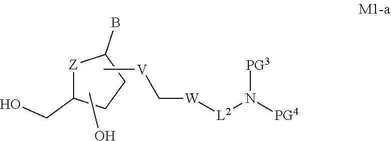

or a pharmaceutically acceptable salt thereof, wherein:
B is a nucleobase or hydrogen;
PG³ and PG⁴ are independently hydrogen or a suitable nitrogen protecting group, provided both PG³ and PG⁴ are not hydrogen at the same time;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

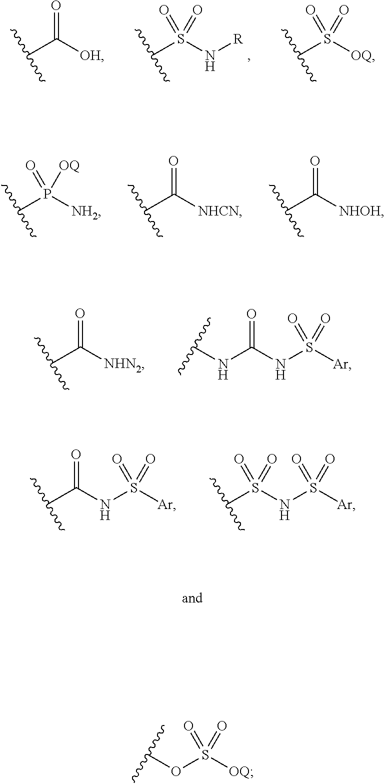

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, a compound of formula M1 is not

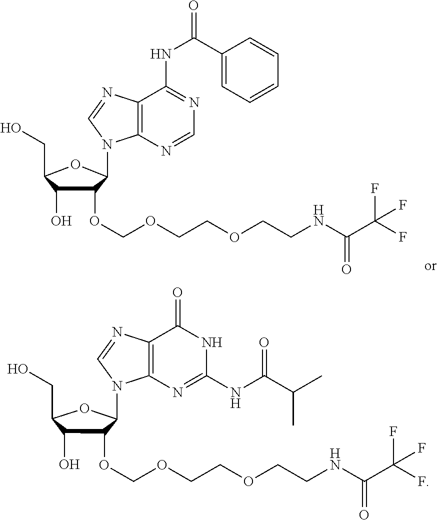

In certain embodiments, the present invention provides a compound of formula M2:

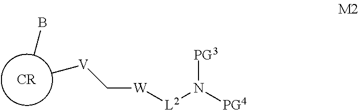

or a pharmaceutically acceptable salt thereof, wherein:

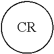

is

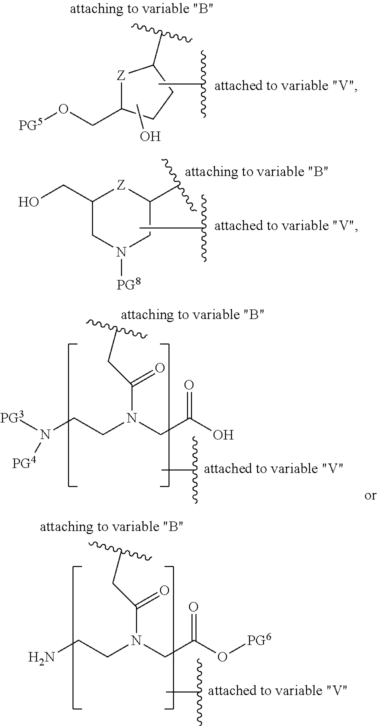

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
PG⁶ is hydrogen or a suitable carboxylate protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

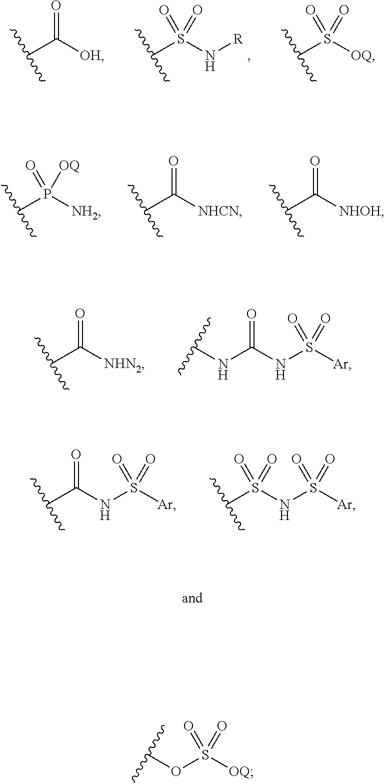

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, the present invention provides a compound of formula M2-a:

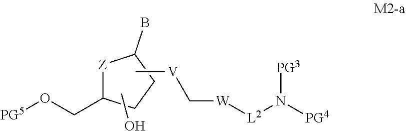

or a pharmaceutically acceptable salt thereof, wherein:
PG³ and PG⁴ are independently hydrogen or a suitable nitrogen protecting group, provided both PG³ and PG⁴ are not hydrogen at the same time;
PG⁵ is a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

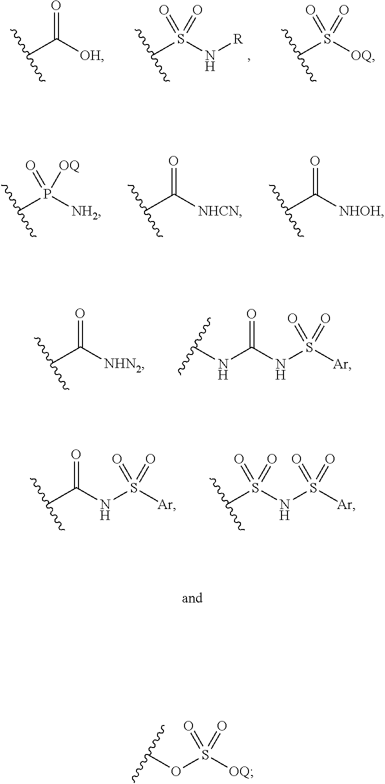

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V is —O—, —S—, or —NR—;
each R is independently selected from hydrogen, alkyl, alkenyl, aromatic, heterocycle, substituted alkyl, and substituted alkenyl; and
Z is —CH₂—, —O—, —S—, or —NR—.

In certain embodiments, a compound of formula M2 is not

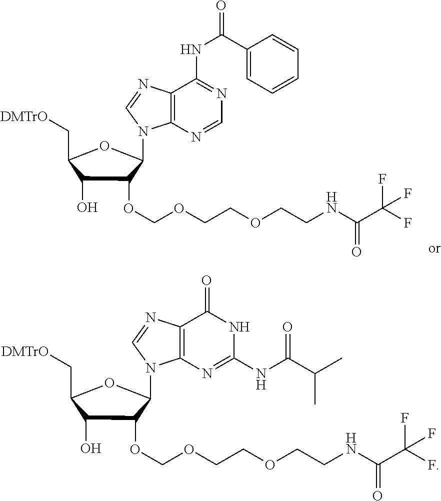

In certain embodiments, the present invention provides a compound of formula M3:

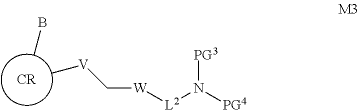

or a pharmaceutically acceptable salt thereof, wherein:

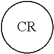

is

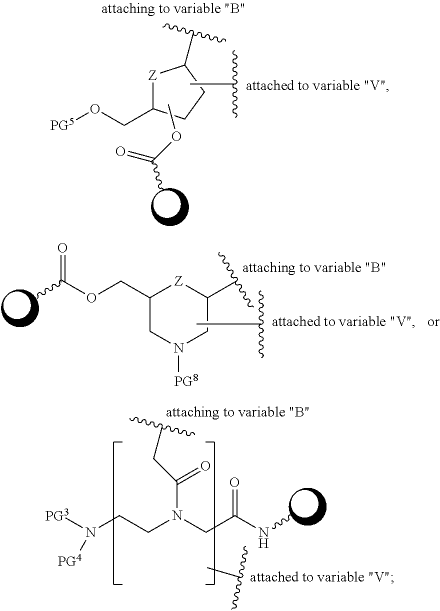

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

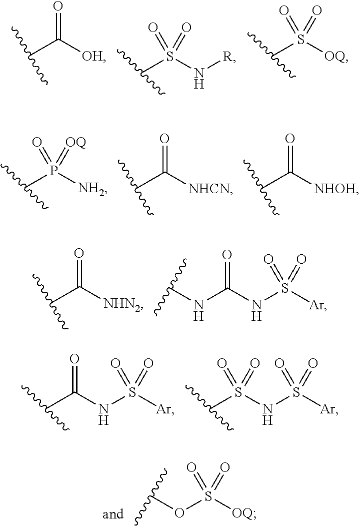

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula M3-a:

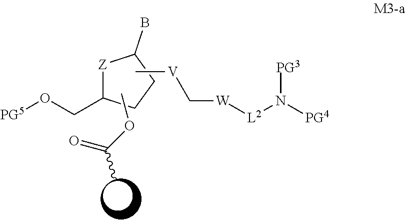

or a pharmaceutically acceptable salt thereof, wherein:
B is a nucleobase or hydrogen;
PG³ and PG⁴ are independently hydrogen or a suitable nitrogen protecting group, provided both PG³ and PG⁴ are not hydrogen at the same time;
PG⁵ is a suitable hydroxyl protecting group;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

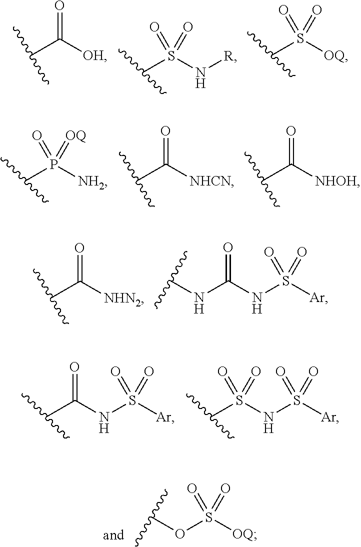

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula M4:

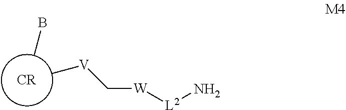

or a pharmaceutically acceptable salt thereof, wherein:

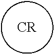

is

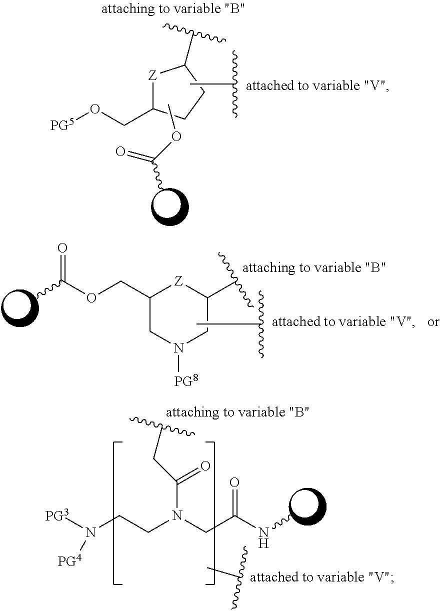

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

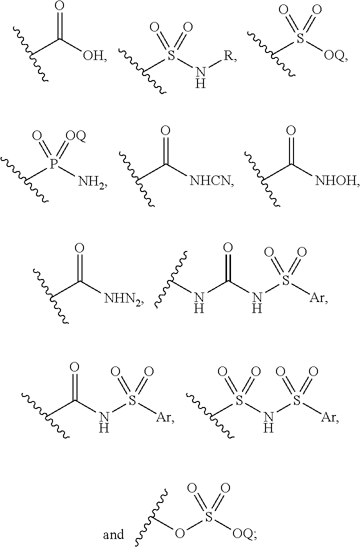

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula M4-a:

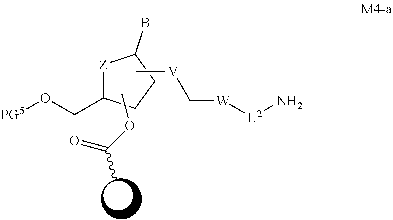

or a pharmaceutically acceptable salt thereof, wherein:
PG⁵ is a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

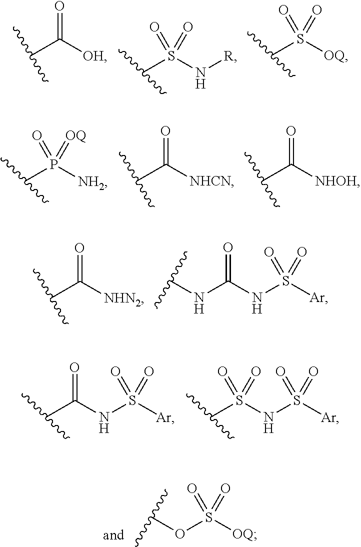

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula P1:

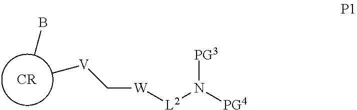

or a salt thereof, wherein:

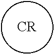

is

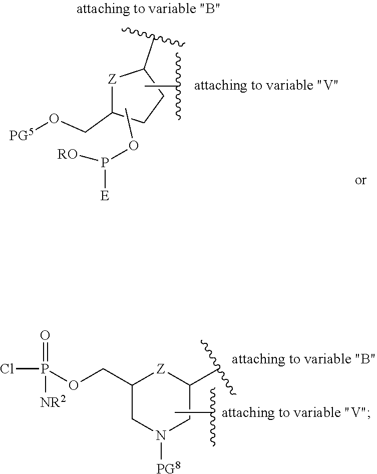

PG³, PG⁴, and PG⁸ are independently hydrogen or a suitable nitrogen protecting group, provided both PG³ and PG⁴ on the same nitrogen are not hydrogen at the same time;
PG⁵ is hydrogen or a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including OH

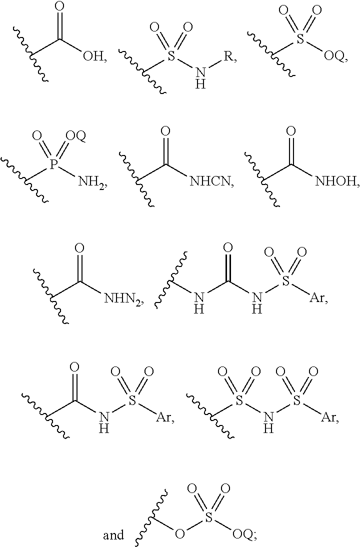

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a compound of formula P1-a:

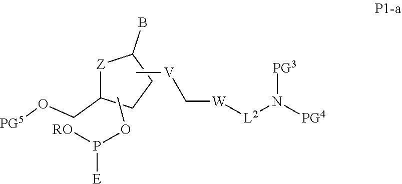

or a salt thereof, wherein:
PG³ and PG⁴ are independently hydrogen or a suitable nitrogen protecting group, provided both PG³ and PG⁴ are not hydrogen at the same time;
PG⁵ is a suitable hydroxyl protecting group;
B is a nucleobase or hydrogen;
E is a halogen or NR₂;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

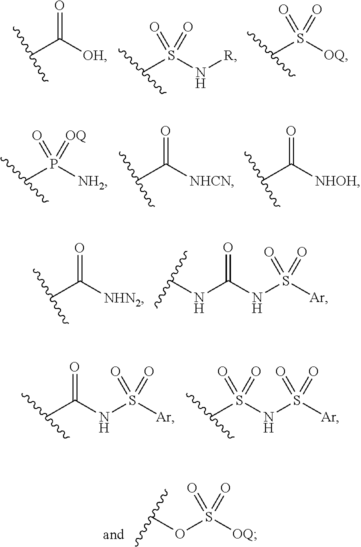

each R is independently selected from hydrogen, alkyl, alkenyl, aromatic, heterocycle, substituted alkyl, and substituted alkenyl, or:
   two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, a compound of formula P1 is not

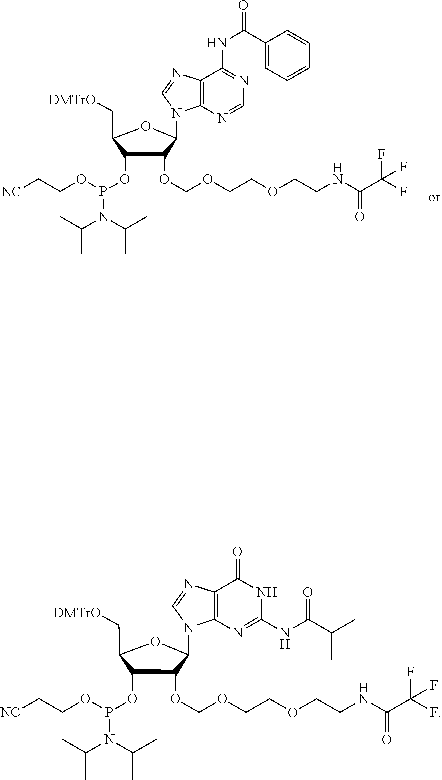

In certain embodiments, the present invention provides a nucleic acid or analogue thereof P2, or a pharmaceutically acceptable salt thereof, comprising

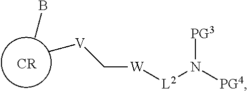

wherein:

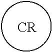

is

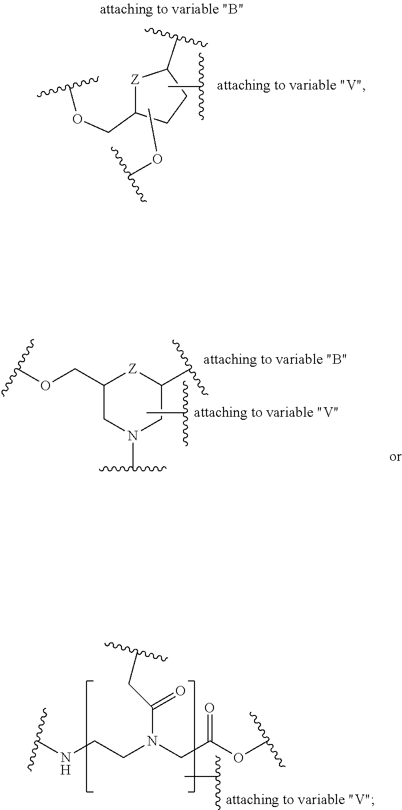

PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

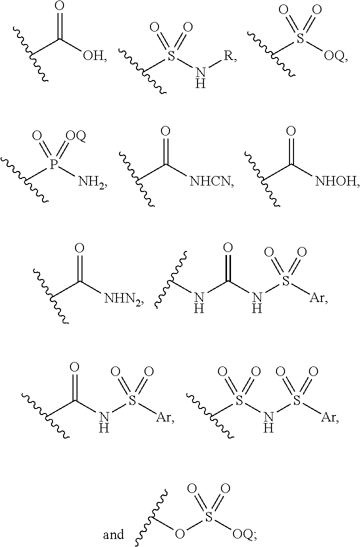

each R is independently selected from hydrogen, alkyl, alkenyl, aromatic, heterocycle, substituted alkyl, and substituted alkenyl, or:
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a nucleic acid or analogue thereof P2-a, or a pharmaceutically acceptable salt thereof, comprising:

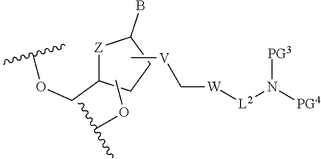

wherein
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

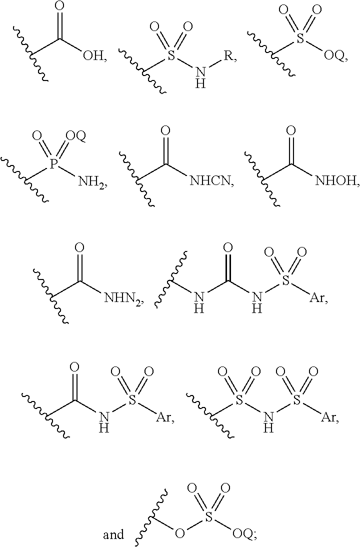

each R is independently selected from hydrogen, alkyl, alkenyl, aromatic, heterocycle, substituted alkyl, and substituted alkenyl, or:
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a nucleic acid or analogue thereof P3, or a pharmaceutically acceptable salt thereof, comprising

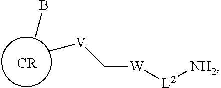

wherein:

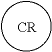

is

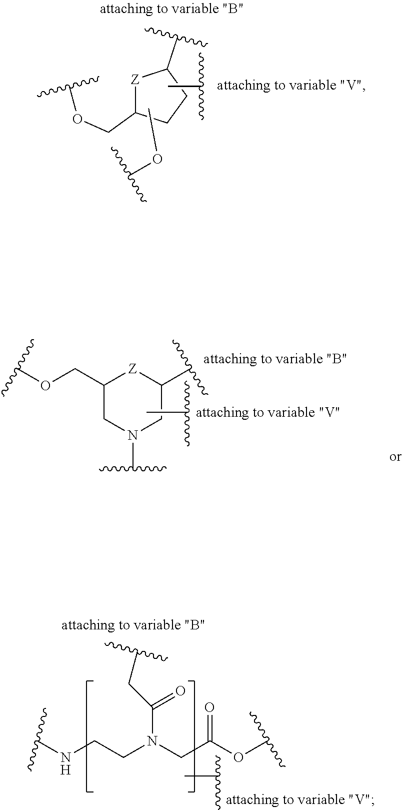

B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

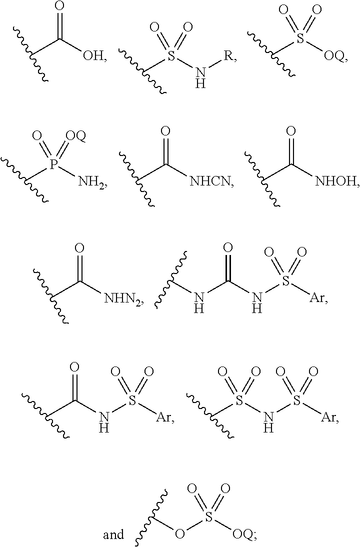

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a nucleic acid or analogue thereof P3-a, or a pharmaceutically acceptable salt thereof, comprising:

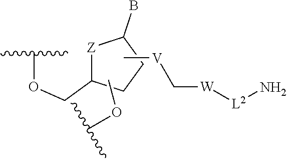

wherein:
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

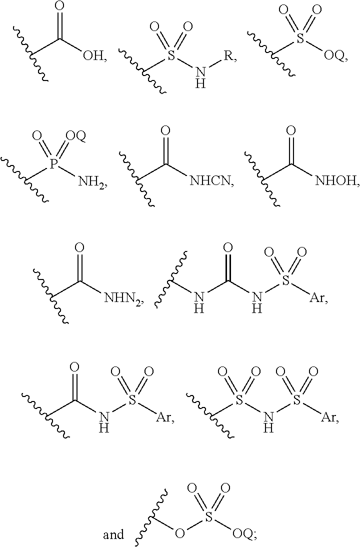

each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

In certain embodiments, the present invention provides a nucleic acid or analogue thereof P4, or a pharmaceutically acceptable salt thereof, comprising

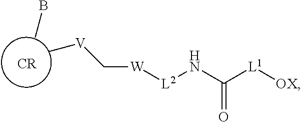

wherein

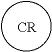

is

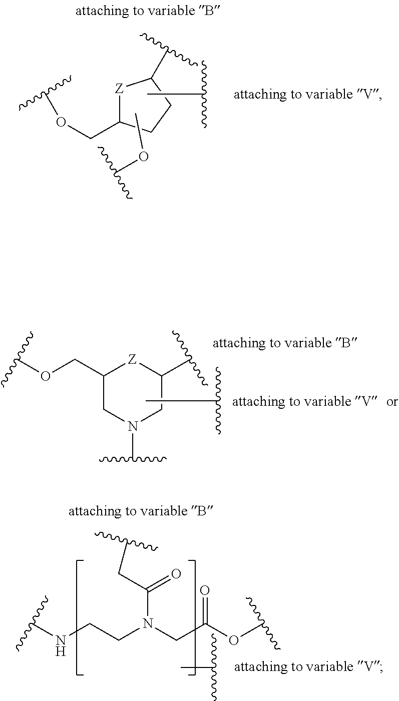

B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

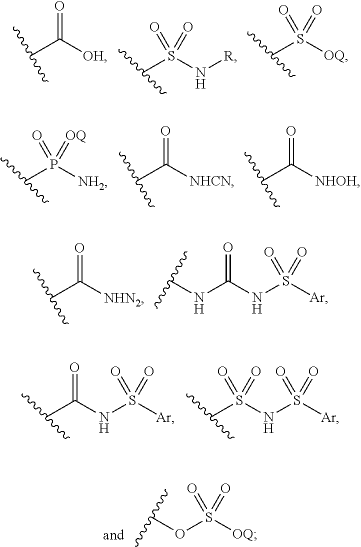

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

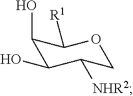

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, the present invention provides a nucleic acid or analogue thereof P4-a, or a pharmaceutically acceptable salt thereof, comprising:

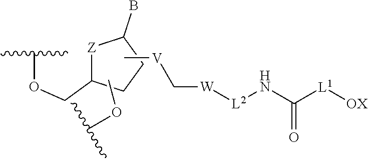

wherein:
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including

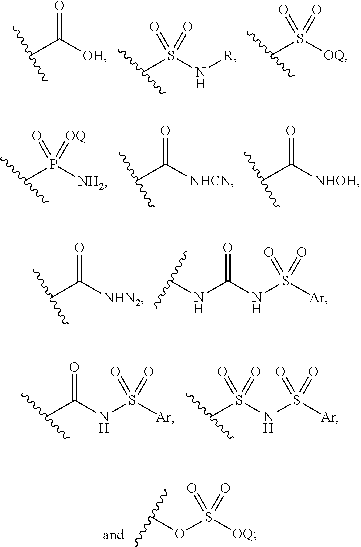

each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and

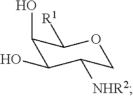

R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—.

In certain embodiments, a nucleic acid or analogue thereof P2, P3, or P4, or a pharmaceutically acceptable salt thereof, is attached to a solid support. In certain embodiments, a nucleic acid or analogue thereof P2, P3, or P4, or a pharmaceutically acceptable salt thereof, is not attached to a solid support.

As defined above and described herein, PG¹, PG² and PG⁵ are independently hydrogen or a suitable hydroxyl protecting group.

In some embodiments, PG¹, PG² and PG⁵ are independently hydrogen. In some embodiments, PG¹, PG² and PG⁵ are independently a suitable hydroxyl protecting group.

As defined above and described herein, PG³ and PG⁴ are independently hydrogen or a suitable nitrogen protecting group

In some embodiments, PG³ and PG⁴ are independently hydrogen. In some embodiments, PG³ and PG⁴ are independently a suitable nitrogen protection group. In some embodiments, both PG³ and PG⁴ are not hydrogen at the same time.

As defined above and described herein, PG⁶ is independently hydrogen or a suitable carboxylate protecting group.

In some embodiments, PG⁶ is independently hydrogen. In some embodiments, PG⁶ is a suitable carboxylate protecting group.

As defined above and described herein, B is a nucleobase or hydrogen.

In some embodiments, B is a nucleobase. In some embodiments, B is a hydrogen.

As defined above and described herein, E is a halogen or NR₂.

In some embodiments, E is a halogen, such as chloro. In some embodiments, E is NR₂.

As defined above and described herein, each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY).

In some embodiments, each L¹ and L² are independently alkyl. In some embodiments, each L¹ and L² are independently alkenyl. In some embodiments, each L¹ and L² are independently alkynyl. In some embodiments, each L¹ and L² are independently aromatic. In some embodiments, each L¹ and L² are independently heterocycle. In some embodiments, each L¹ and L² are independently substituted alkyl. In some embodiments, each L¹ and L² are independently substituted alkenyl. In some embodiments, each L¹ and L² are independently substituted alkynyl. In some embodiments, one or more methylenes of each L¹ and L² are can be independently interrupted or terminated by one or more of P(O)H, P(O₂), P(O₄), polyethylenegylcol (PEG), OY, S, S(OY), SO₂(Y), (C═O)OY, NY₂, NH, and NH—(C═OY).

As defined above and described herein each Y is independently selected from H, C₁-C₆ alkanyl, C₁-C₆ alkenyl or aryl, including

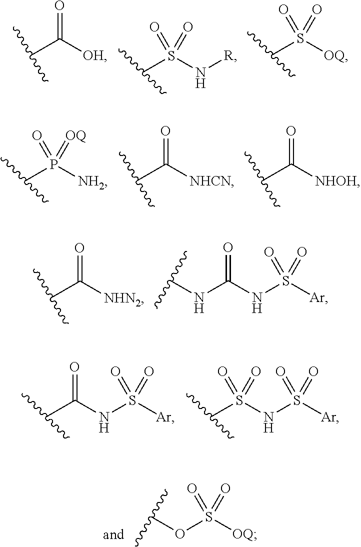

In some embodiments, Y is independently selected from H. In some embodiments, Y is independently selected from C₁-C₆ alkanyl. In some embodiments, Y is independently selected from C₁-C₆ alkenyl. In some embodiments, Y is independently selected from aryl. In some embodiments, Y is independently selected from

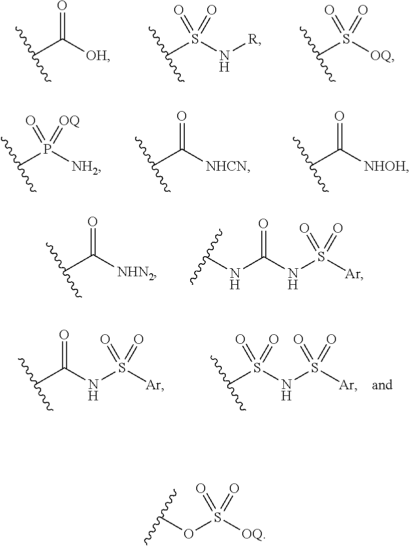

As defined above and described herein, each R is independently selected from hydrogen, alkyl, alkenyl, aromatic, heterocycle, substituted alkyl, or substituted alkenyl, or two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur.

In some embodiments, R is hydrogen. In some embodiments, R is alkyl. In some embodiments, R is alkenyl. In some embodiments, R is aromatic. In some embodiments, R is heterocycle. In some embodiments, R is substituted alkyl. In some embodiments, R is substituted alkenyl. In some embodiments, two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur.

As defined above and described herein, Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY.

In some embodiments, Q is H. In some embodiments, Q is a pharmaceutically acceptable salt. In some embodiments, Q is C₁-C₆ alkanyl. In some embodiments, Q is C₁-C₆ alkenyl. In some embodiments, Q is C₁-C₆ alkynyl. In some embodiments, Q is aryl. In some embodiments, Q is heteroaryl. In some embodiments, Q is (CH₂)ₘ-aryl. In some embodiments, Q is (CH₂)ₘ-heteroaryl. In some embodiments, m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY.

In some embodiments, L¹ is the same as L^{1′}. In some embodiments, L¹ is —CH₂-L^{1′}.

As defined above and described herein, X is a ligand selected from GalNAc, D-mannose, L-galactose, D-arabinose, L-fucose, polyols, and

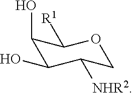

In some embodiments, X is GalNAc. In some embodiments, X is D-mannose. In some embodiments, X is L-galactose. In some embodiments, X is D-arabinose. In some embodiments, X is L-fucose. In some embodiments, X is polyols. In some embodiments, X is

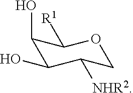

As defined above and described herein, R¹ is selected from CF₃, alkyl, alkenyl, alkynyl, aromatic, heterocycle, substituted alkyl, substituted alkenyl, and substituted alkynyl.

In some embodiments, R¹ is CF₃. In some embodiments, R¹ is alkyl. In some embodiments, R¹ is alkenyl. In some embodiments, R¹ is alkynyl. In some embodiments, R¹ is aromatic. In some embodiments, R¹ is heterocycle. In some embodiments, R¹ is substituted alkyl. In some embodiments, R¹ is substituted alkenyl. In some embodiments, R¹ is substituted alkynyl.

As defined above and described herein, R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³).

In some embodiments, R² is one or more methylenes interrupted or terminated by one or more of P(O)H, P(O₂), P(O₄), polyethylenegylcol (PEG), OR³, S, S(OR³), SO₂(R³), (C═O)OR³, NY₂, NH, or NH(C═OR³).

As defined above and described herein, R³ is H, C₁-C₆ alkanyl, C₁-C₆ alkenyl, or aryl.

In some embodiments, R³ is H. In some embodiments, R³ is C₁-C₆ alkanyl. In some embodiments, R³ is C₁-C₆ alkenyl. In some embodiments, R³ is aryl.

As defined above and described herein, V is —O—, —S—, or —NR—.

In some embodiments, V is —O—. In some embodiments, V is —S—. In some embodiments, V is —NR—.

As defined above and described herein, W is —O—, —S—, or —NR—.

In some embodiments, W is —O—. In some embodiments, W is —S—. In some embodiments, W is —NR—.

As defined above and described herein, Z is —CH₂—, —O—, —S—, or —NR—.

In some embodiments, Z is —CH₂—. In some embodiments, Z is —O—. In some embodiments, Z is —S—. In some embodiments, Z is —NR—.

In certain embodiments, the present invention provides a compound of formula F-6-a wherein W is —O—, thereby providing a compound of formula F-6-b:

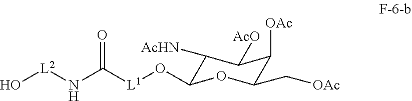

or a pharmaceutically acceptable salt thereof.

In certain embodiments, the present invention provides a compound of formula F-6-a wherein L¹ is

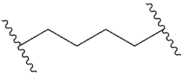 [SMILES: *CCCC*]

### and L² is

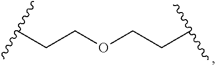 [SMILES: *CCOCC*]

thereby providing a compound of formula F-6-c:

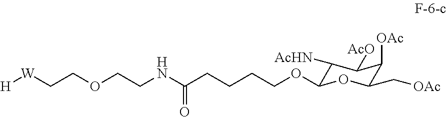

or a pharmaceutically acceptable salt thereof.

In certain embodiments, the present invention provides a compound of formula F-6-a wherein L¹ is

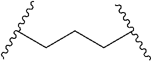 [SMILES: *CCC*]

### and L² is

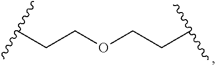 [SMILES: *CCOCC*]

thereby providing a compound of formula F-6-d:

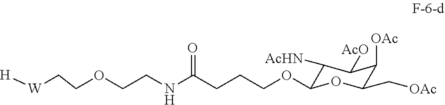

or a pharmaceutically acceptable salt thereof.

In certain embodiments, the present invention provides a compound of formula D wherein X is GalNAc, L¹ is

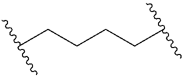 [SMILES: *CCCC*]

### and L² is

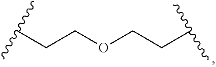 [SMILES: *CCOCC*]

thereby providing a compound of formula D-c:

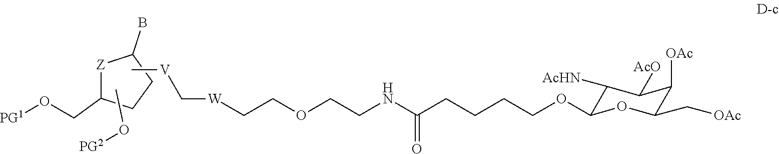

or a pharmaceutically acceptable salt thereof.

In certain embodiments, the present invention provides a compound of formula D wherein X is GalNAc, L¹ is

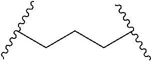 [SMILES: *CCC*]

### and L² is

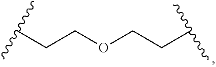 [SMILES: *CCOCC*]

thereby providing a compound of formula D-e:

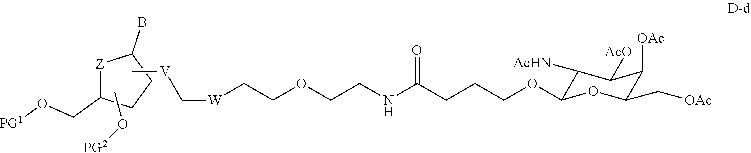

or a pharmaceutically acceptable salt thereof.

In certain embodiments, the present invention provides a compound of formula D wherein X is GalNAc, L¹ is

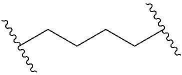 [SMILES: *CCCC*]

### and L² is

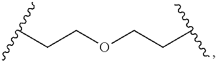 [SMILES: *CCOCC*]

thereby providing a compound of formula D-e:

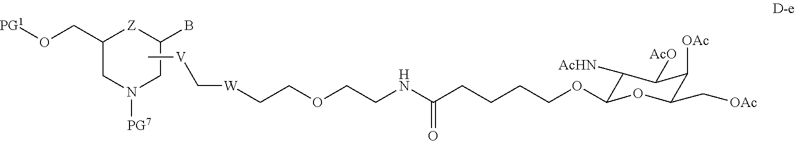

or a pharmaceutically acceptable salt thereof.

In certain embodiments, the present invention provides a compound of formula D wherein X is GalNAc, L¹ is

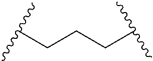 [SMILES: *CCC*]

### and L² is

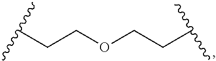 [SMILES: *CCOCC*]

thereby providing a compound of formula D-f:

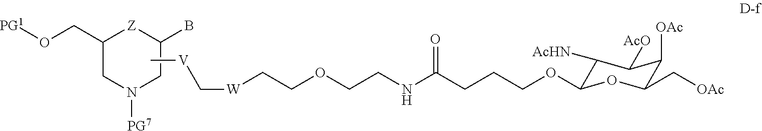

or a pharmaceutically acceptable salt thereof.

In certain embodiments, the present invention provides a compound of formula D wherein X is GalNAc, L¹ is

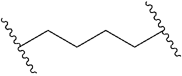 [SMILES: *CCCC*]

### and L² is

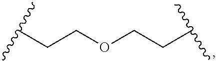 [SMILES: *CCOCC*]

thereby providing a compound of formula D-g:

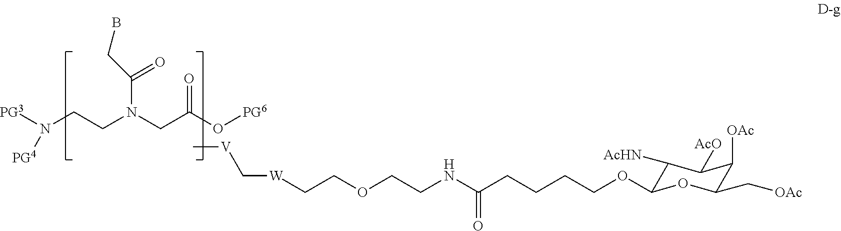

or a pharmaceutically acceptable salt thereof.

In certain embodiments, the present invention provides a compound of formula D wherein X is GalNAc, L¹ is

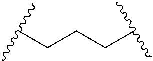 [SMILES: *CCC*]

### and L² is

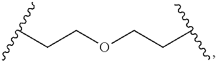 [SMILES: *CCOCC*]

thereby providing a compound of formula D-h:

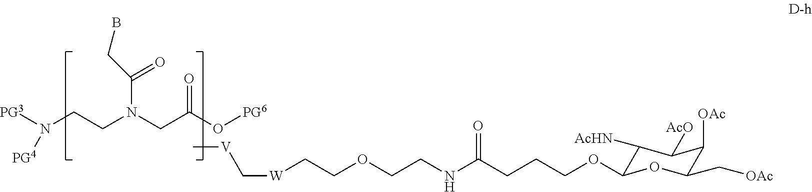

or a pharmaceutically acceptable salt thereof.

In certain embodiments, the present invention provides a compound of formula C wherein X is GalNAc, L¹ is

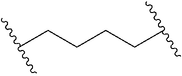 [SMILES: *CCCC*]

### and L² is

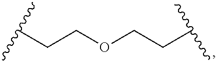 [SMILES: *CCOCC*]

thereby providing a compound of formula C-c:

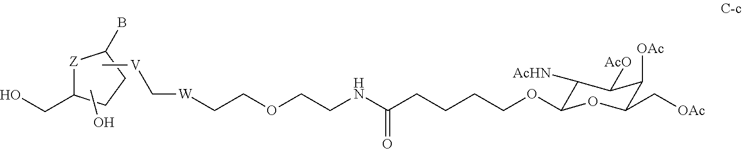

or a pharmaceutically acceptable salt thereof.

In certain embodiments, the present invention provides a compound of formula C wherein X is GalNAc, L¹ is

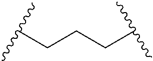 [SMILES: *CCC*]

### and L² is

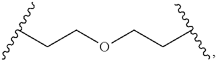 [SMILES: *CCOCC*]

thereby providing a compound of formula C-d:

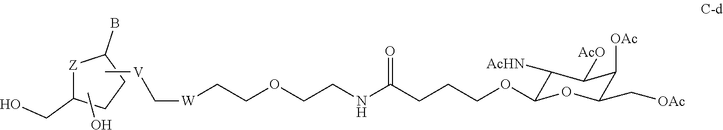

or a pharmaceutically acceptable salt thereof.

In certain embodiments, the present invention provides a compound of formula C wherein X is GalNAc, L¹ is

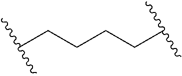 [SMILES: *CCCC*]

### and L² is

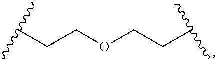 [SMILES: *CCOCC*]

thereby providing a compound of formula C-e:

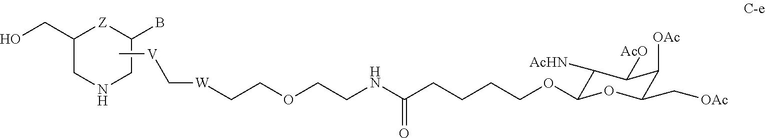

or a pharmaceutically acceptable salt thereof.

In certain embodiments, the present invention provides a compound of formula C wherein X is GalNAc, L¹ is

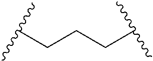 [SMILES: *CCC*]

### and L² is

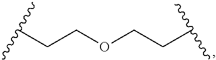 [SMILES: *CCOCC*]

thereby providing a compound of formula C-f:

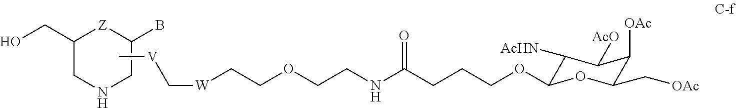

or a pharmaceutically acceptable salt thereof.

In certain embodiments, the present invention provides a compound of formula C wherein X is GalNAc, L¹ is

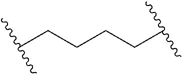 [SMILES: *CCCC*]

### and L² is

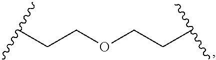 [SMILES: *CCOCC*]

thereby providing a compound of formula C-g-1, C-g-2, or C-g-3:

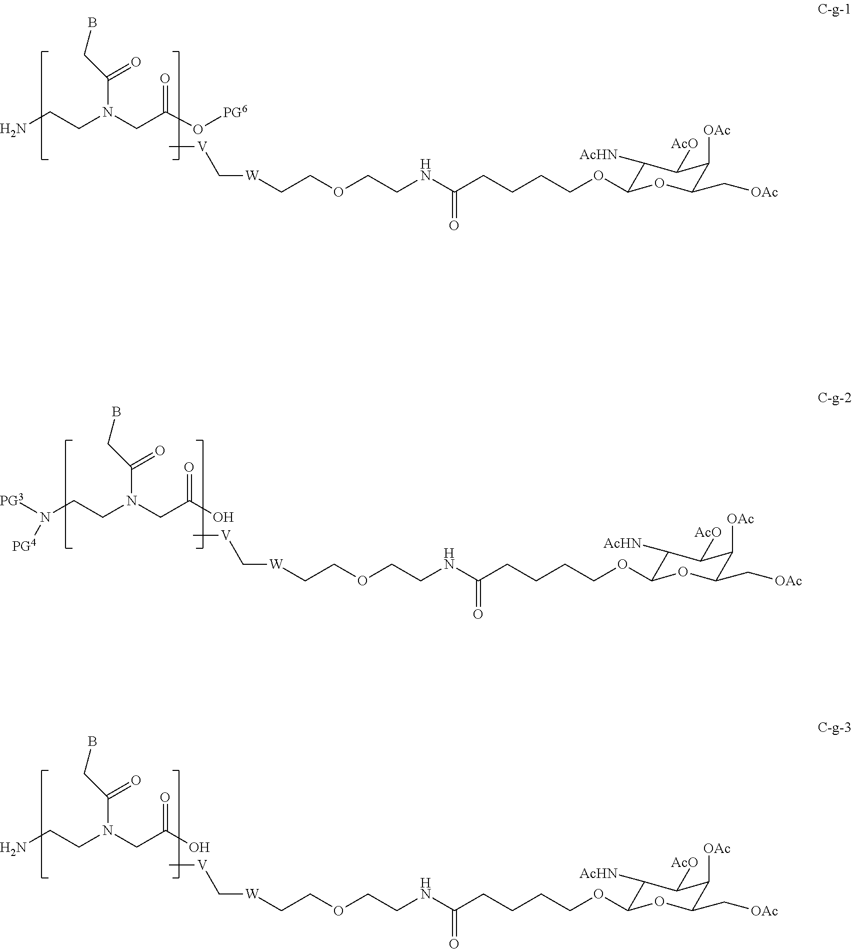

or a pharmaceutically acceptable salt thereof.

In certain embodiments, the present invention provides a compound of formula C wherein X is GalNAc, L¹ is

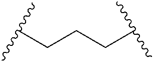 [SMILES: *CCC*]

### and L² is

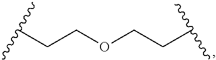 [SMILES: *CCOCC*]

thereby providing a compound of formula C-h-1, C-h-2, or C-h-3:

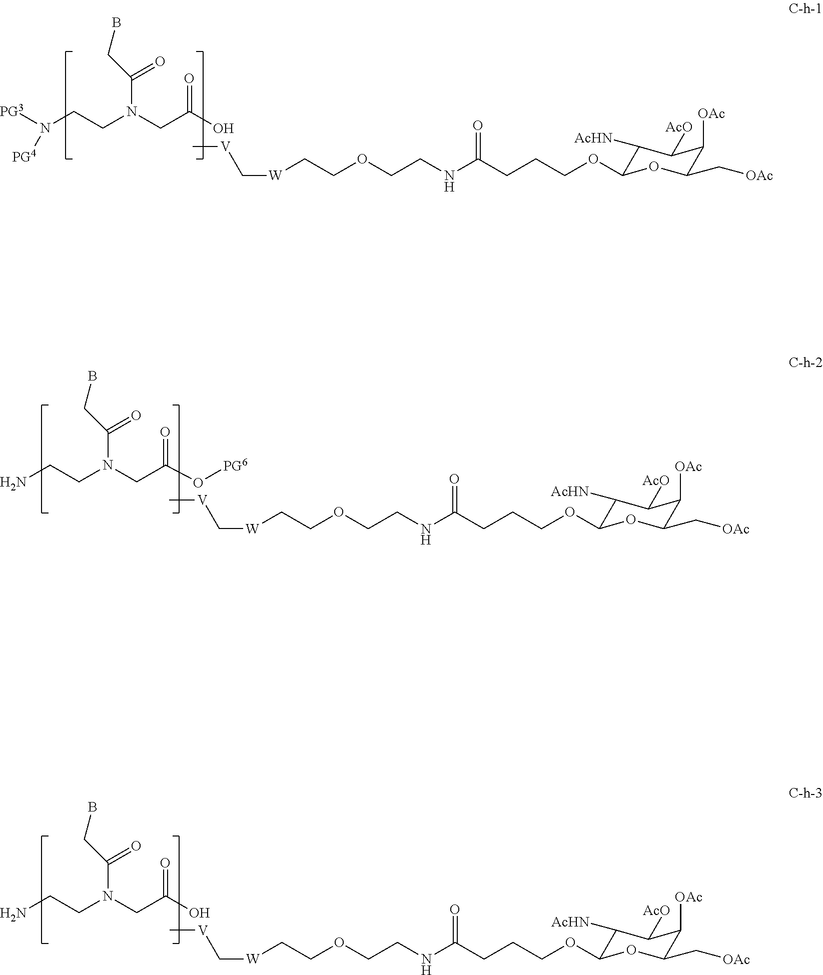

or a pharmaceutically acceptable salt thereof.

In certain embodiments, the present invention provides a compound of formula B wherein X is GalNAc, L¹ is

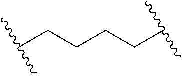 [SMILES: *CCCC*]

### and L² is

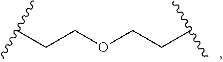 [SMILES: *CCOCC*]

thereby providing a compound of formula B-c:

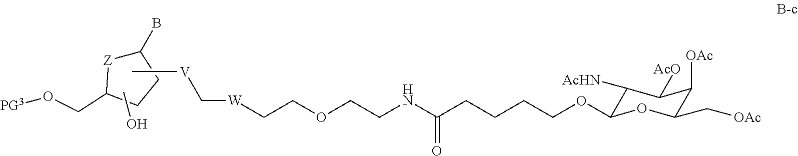

or a pharmaceutically acceptable salt thereof.

In certain embodiments, the present invention provides a compound of formula B wherein X is GalNAc, L¹ is

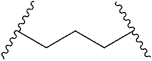 [SMILES: *CCC*]

### and L² is

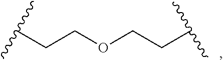 [SMILES: *CCOCC*]

thereby providing a compound of formula B-d:

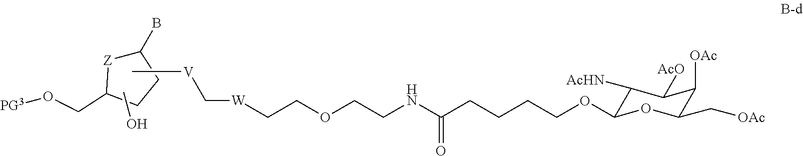

or a pharmaceutically acceptable salt thereof.

In certain embodiments, the present invention provides a compound of formula B wherein X is GalNAc, L¹ is

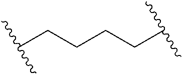 [SMILES: *CCCC*]

### and L² is

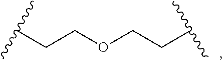 [SMILES: *CCOCC*]

thereby providing a compound of formula B-e:

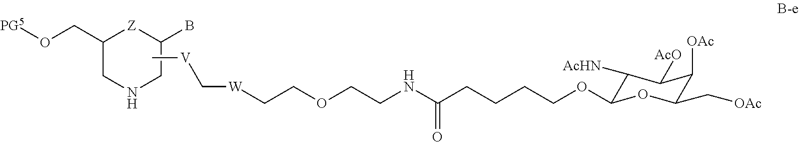

or a pharmaceutically acceptable salt thereof.

In certain embodiments, the present invention provides a compound of formula B wherein X is GalNAc, L¹ is

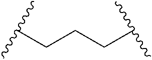 [SMILES: *CCC*]

### and L² is

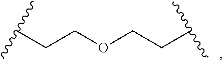 [SMILES: *CCOCC*]

thereby providing a compound of formula B-f:

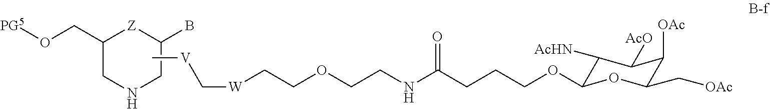

or a pharmaceutically acceptable salt thereof.

In certain embodiments, the present invention provides a compound of formula A wherein X is GalNAc, L¹ is

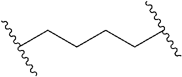 [SMILES: *CCCC*]

### and L² is

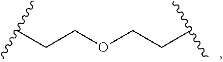 [SMILES: *CCOCC*]

thereby providing a compound of formula A-c:

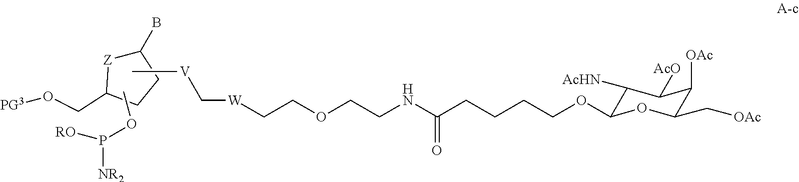

or a pharmaceutically acceptable salt thereof.

In certain embodiments, the present invention provides a compound of formula A wherein X is GalNAc, L¹ is

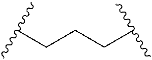 [SMILES: *CCC*]

### and L² is

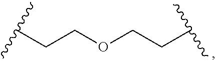 [SMILES: *CCOCC*]

thereby providing a compound of formula A-d:

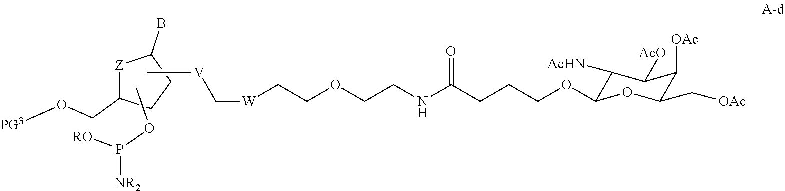

or a pharmaceutically acceptable salt thereof.

In certain embodiments, the present invention provides a compound of formula A wherein X is GalNAc, L¹ is

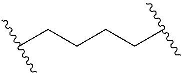 [SMILES: *CCCC*]

### and L² is

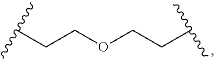 [SMILES: *CCOCC*]

thereby providing a compound of formula A-e:

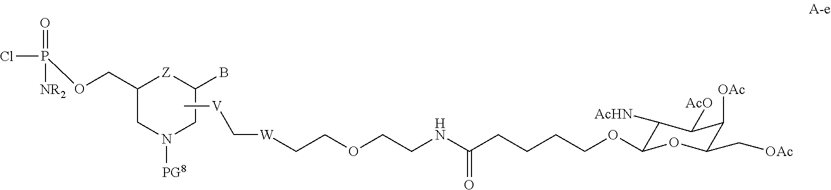

or a pharmaceutically acceptable salt thereof.

In certain embodiments, the present invention provides a compound of formula A wherein X is GalNAc, L¹ is

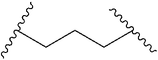 [SMILES: *CCC*]

### and L² is

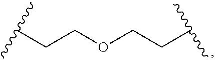 [SMILES: *CCOCC*]

thereby providing a compound of formula A-f:

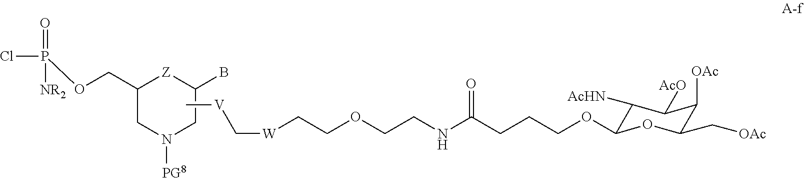

or a pharmaceutically acceptable salt thereof.

In certain embodiments, the present invention provides a compound of formula A1 wherein X is GalNAc, L¹ is

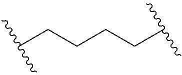 [SMILES: *CCCC*]

### and L² is

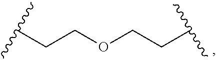 [SMILES: *CCOCC*]

thereby providing a compound of formula A1-c:

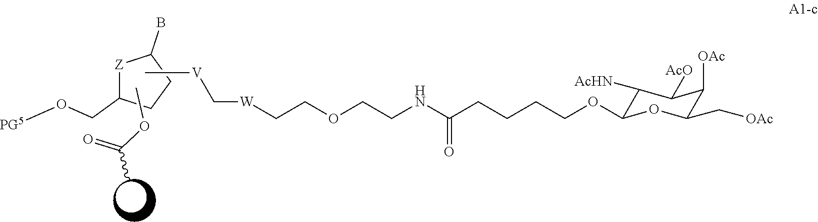

or a pharmaceutically acceptable salt thereof.

In certain embodiments, the present invention provides a compound of formula A1 wherein X is GalNAc, L¹ is

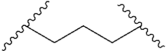 [SMILES: *CCC*]

### and L² is

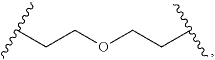 [SMILES: *CCOCC*]

thereby providing a compound of formula A1-d:

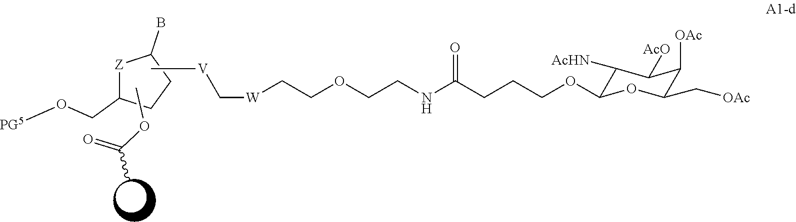

or a pharmaceutically acceptable salt thereof.

In certain embodiments, the present invention provides a compound of formula A1 wherein X is GalNAc, L¹ is

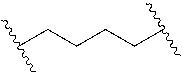 [SMILES: *CCCC*]

### and L² is

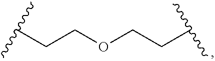 [SMILES: *CCOCC*]

thereby providing a compound of formula A1-e:

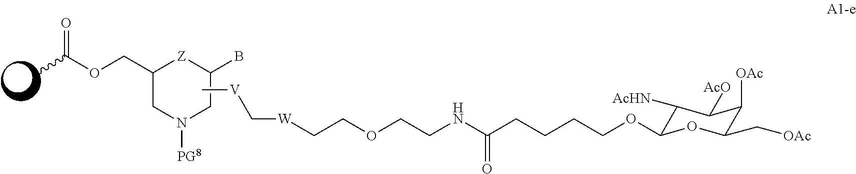

or a pharmaceutically acceptable salt thereof.

In certain embodiments, the present invention provides a compound of formula A1 wherein X is GalNAc, L¹ is

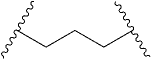 [SMILES: *CCC*]

### and L² is

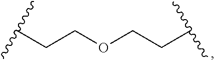 [SMILES: *CCOCC*]

thereby providing a compound of formula A1-f:

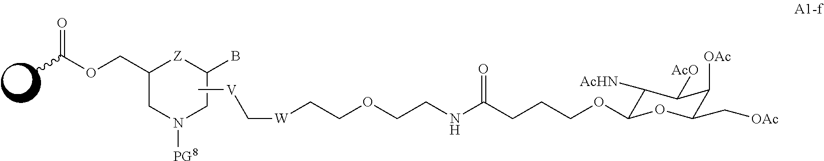

or a pharmaceutically acceptable salt thereof.

In certain embodiments, the present invention provides a compound of formula A1 wherein X is GalNAc, L¹ is

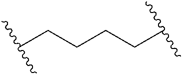 [SMILES: *CCCC*]

### and L² is

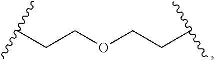 [SMILES: *CCOCC*]

thereby providing a compound of formula A1-g:

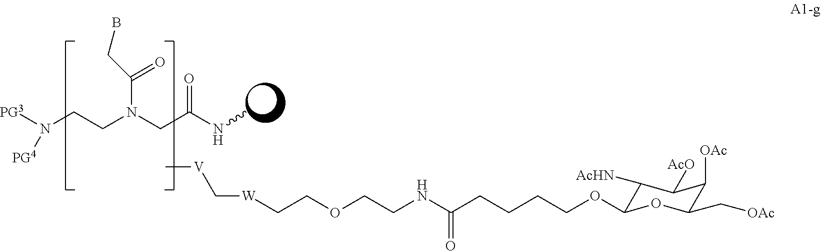

or a pharmaceutically acceptable salt thereof.

In certain embodiments, the present invention provides a compound of formula A1 wherein X is GalNAc, L¹ is

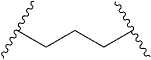 [SMILES: *CCC*]

### and L² is

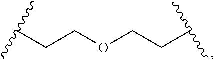 [SMILES: *CCOCC*]

thereby providing a compound of formula A1-h:

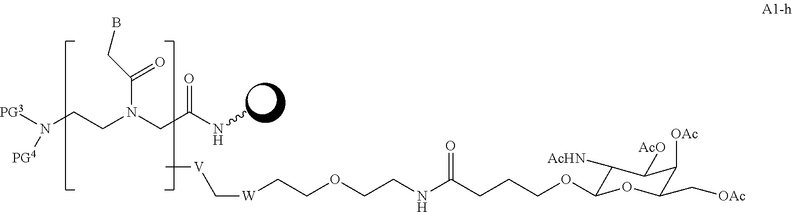

or a pharmaceutically acceptable salt thereof.

As described herein, at step S-5 above, a compound of formula F is treated with an alcohol compound of formula

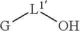

to afford the glycosylation product compound E-a, wherein G is a carboxylic acid having a suitable carboxylate protecting group or a functional group that can be reacted to form a carboxylic acid. In some embodiments, G of an alcohol compound of formula

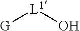

can be an alkenyl group. As described above, when G of an alcohol compound of formula

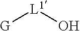

is an alkenyl group

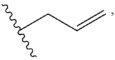 [SMILES: *CC=C]

there can be a double bond migration impurity of formula

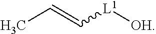

Accordingly, in some embodiments, when G is an alkenyl group

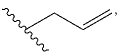 [SMILES: *CC=C]

compound of formula E-a comprises an impurity of formula

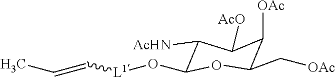

In some embodiments, a compound of formula F-3-a having structure

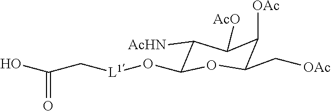

comprises an impurity of formula

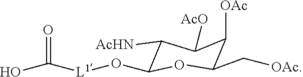

In some embodiments, a compound of formula F-6 having structure

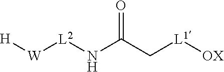

comprises an impurity of formula

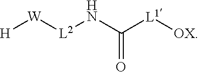

In some embodiments, a compound of formula D having structure

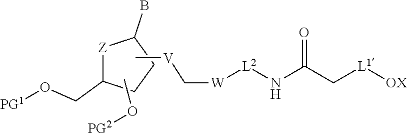

comprises an impurity of formula

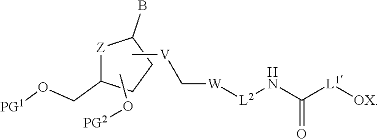

In some embodiments, a compound of formula C having structure

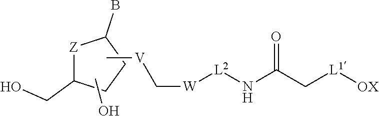

comprises an impurity of formula

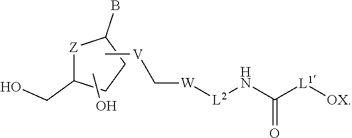

In some embodiments, a compound of formula B having structure

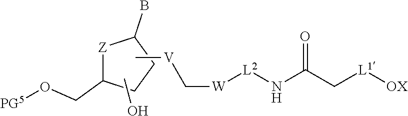

comprises an impurity of formula

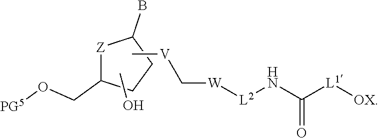

In some embodiments, a compound of formula A having structure

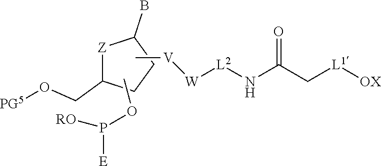

comprises an impurity of formula

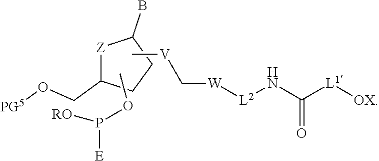

In some embodiments, a compound of formula A1 having structure

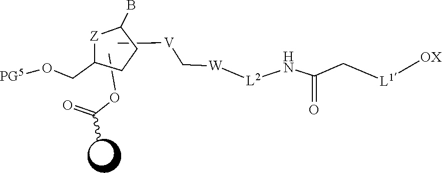

comprises an impurity of formula

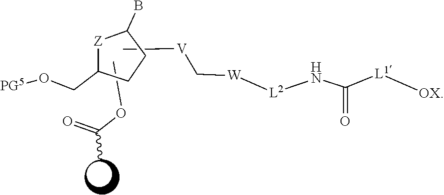

A compound of formula A can be used in synthesis of a nucleic acid or analogue thereof comprising one or more GalNAc ligand. As a compound of formula A can comprise an impurity with one less methylene unit at position L¹ (i.e., an impurity with molecular weight of M-14), a nucleic acid or analogue thereof prepared using a compound of formula A can comprise a corresponding M-14 nucleic acid or analogue thereof impurity for each GalNAc ligand incorporated. Accordingly, the present invention provides a composition comprising a nucleic acid or analogue thereof comprising t times GalNAc ligands, and nucleic acid or analogue thereof impurities of molecular weight of M-14, M-(14×2), . . . and M-(14×t). In some embodiments, a nucleic acid or analogue thereof is attached to a solid support. In some embodiments, a nucleic acid or analogue thereof is not attached to a solid support.

In some embodiments, the present invention provides a composition comprising a nucleic acid or analogue thereof comprising one GalNAc ligand, and a nucleic acid or analogue thereof impurity with molecular weight of M-14 (i.e., having one less methylene unit at position L¹ of the GalNAc ligand).

In some embodiments, the present invention provides a composition comprising a nucleic acid or analogue thereof comprising two GalNAc ligands, a nucleic acid or analogue thereof impurity with molecular weight of M-14 (i.e., having one less methylene unit at position L¹ for either of the GalNAc ligands), and a nucleic acid or analogue thereof impurity with molecular weight of M-28 (i.e., having one less methylene unit at position L¹ for each of the GalNAc ligands).

In some embodiments, the present invention provides a composition comprising a nucleic acid or analogue thereof comprising three GalNAc ligands, a nucleic acid or analogue thereof impurity with molecular weight of M-14 (i.e., having one less methylene unit at position L¹ for one of the GalNAc ligands), a nucleic acid or analogue thereof impurity with molecular weight of M-28 (i.e., having one less methylene unit at position L¹ for two of the GalNAc ligands), and a nucleic acid or analogue thereof impurity with molecular weight of M-42 (i.e., having one less methylene unit at position L¹ for each of the GalNAc ligands).

In some embodiments, the present invention provides a composition comprising a nucleic acid or analogue thereof comprising four GalNAc ligands, a nucleic acid or analogue thereof impurity with molecular weight of M-14 (i.e., having one less methylene unit at position L¹ for one of the GalNAc ligands), a nucleic acid or analogue thereof impurity with molecular weight of M-28 (i.e., having one less methylene unit at position L¹ for two of the GalNAc ligands), a nucleic acid or analogue thereof impurity with molecular weight of M-42 (i.e., having one less methylene unit at position L¹ for three of the GalNAc ligands), and a nucleic acid or analogue thereof impurity with molecular weight of M-56 (i.e., having one less methylene unit at position L¹ for each of the GalNAc ligands).

In some embodiments, the present invention provides a double stranded nucleic acid (dsNA) as described in US 20170305956, the content of which is incorporated herein by reference in its entirety, which further comprises a corresponding M-14 nucleic acid or analogue thereof impurity for each GalNAc ligand incorporated. In some embodiments, the present invention provides a composition comprising a dsNA comprising t times GalNAc ligands, and dsNA impurities of molecular weight of M-14, M-(14×2), . . . and/or M-(14×t). In some embodiments, the present invention provides a composition comprising a dsNA, wherein the sense strand comprises t times GalNAc ligands, and dsNA impurities wherein the sense strands are of molecular weight of M-14, M-(14×2), . . . and/or M-(14×t).

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
L-DBTA: dibenzoyl-L-tartaric acid
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-rmethylnorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

As depicted in the Examples below, in certain exemplary embodiments, compounds were prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1. Synthesis of 5-(((2R,3R,4R,5R,6R)-3-acetamido-4,5-diacetoxy-6-(acetoxymethyl)tetrahydro-2H-pyran-2-yl)oxy)pentanoic acid (1)

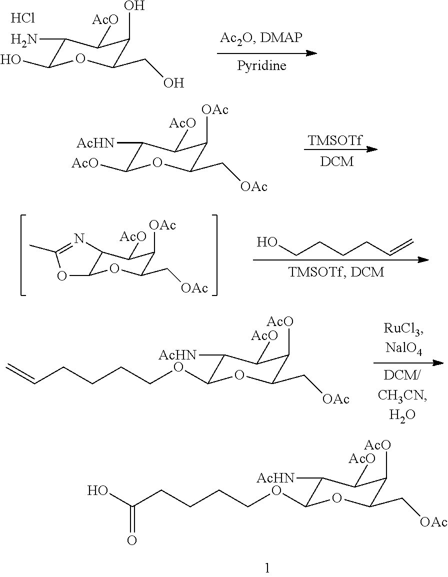

### Step 1: (2S,3R,4R,5R,6R)-3-acetamido-6-(acetoxymethyl)tetrahydro-2H-pyran-2,4,5-triyl triacetate

Pyridine (10.0 eq), DMAP (0.02 eq) and D-galactosamine hydrochloride (1.0 eq) were charged to a reactor and cooled to 5±5° C. Addition of Ac₂O (7.0 eq) was added dropwise to the reactor at 5±5° C. and the reactor was warmed to 35±5° C. carefully and stirred for at least 18 hours at 35±5° C. HPLC analysis was performed every 2 hours until area % of D-galactosamine hydrochloride is not more than 3% and area % of intermediate (RRT=0.80) is not more than 3%. Thereafter the system was then cooled to 5±5° C. and charged with soft water (12.0V) to the reactor at 5±5° C. Stirring was performed for at least 1 hour at 20±5° C., followed by centrifuge and collection of the cake. The filter cake was then slurried with soft water (5V×3), followed by centrifuge and collection the cake. The filter cake was then slurried with MTBE (2.5V), followed by centrifuge and collection the cake. The filter cake was dried under vacuum for at least 12 hours at 40±5° C. until LOD≤5% and packaged in double LDPE bags and stored at room temperature.

### Step 2: (2R,3R,4R,5R,6R)-5-acetamido-2-(acetoxymethyl)-6-(hex-5-en-1-yloxy)tetrahydro-2H-pyran-3,4-diyl diacetate

DCM (6.0V) and (2S,3R,4R,5R,6R)-3-acetamido-6-(acetoxymethyl)tetrahydro-2H-pyran-2,4,5-triyl triacetate (1.0 eq) were charged to a reactor. Water content was analyzed and if water content was >0.1%, the mixture was repeatedly concentrated under vacuum and diluted with DCM (3.0V) until the system was ≤3.0V until the water content was ≤0.1%. TMSOTf (1.5 eq) was then added dropwise to the mixture at 20-30° C. and the system was stirred for at least 2 hours at 20-30° C. Reaction progress was monitored by TLC. Afterward the system was quenched by the dropwise addition to a 5% NaHCO₃ solution (10.0V). The mixture was then stirred for at least 30 min, separated, and the organic phase was collected. The aqueous was extracted with DCM (3.0V) aqueous phase, and after stirring for 30 min was filtered and the filter cake rinsed with DCM (2.0V). The filtrate was then separated and the organic phase collected. The organic phases were combined and concentrated under vacuum below 40° C. until the system was ≤3.0V. DCM (3.0V) was then charged to the mixture and water content was analyzed and if water content was >0.05%, the mixture was repeatedly concentrated under vacuum and diluted with DCM (3.0V) until the system was ≤3.0V until the water content was ≤0.05%. Thereafter, 5-hexen-1-ol was charged into the mixture and the mixture was cooled to 0-5° C. TMSOTf (0.5 eq) was then added dropwise to the mixture at 0-5° C. and the mixture was stirred for 0.5 h at 0-5° C., warmed to 20-30° C., and stirred for at least 2 h. The reaction mixture was then quenched with soften water (10.0V), stirred for at least 0.5 h, separated and the organic phase collected. The organic phase was washed with 8% NaCl solution (10.0V×1) and concentrated under vacuum below 45° C. until the system was 1.0V-1.5V. The organic phase was then filtered through silica gel column (1 wt) and eluted with EA/n-Heptane (1:1). The resulting organic phase was concentrated below 45° C. under vacuum to ≤3.0V. DCM (3.0V) was charged to the mixture and concentrated until the system was ≤3.0V, twice. MTBE (3.0V) was charged to the mixture and concentrated until the system was ≤3.0V, thrice. n-Heptane (1.0V) was then added dropwise into the mixture at a controlled temperature of 20±5° C. The mixture was then cooled to 0-5° C. and stir for at least 2 h. The mixture was centrifuged and the cake was rinsed with n-Heptane (1.0V) and collected. The filter cake was then slurried in n-Heptane (3.0V) for at least 2 h at 15±5° C. The mixture was again centrifuged and the cake was rinsed with n-Heptane (1.0V) and collected. The filter cake was then dried under vacuum for at least 12 hours at 30±5° C. until LOD ≤3% and packaged in double LDPE bags and stored at room temperature.

### Step 3: 5-(((2R,3R,4R,5R,6R)-3-acetamido-4,5-diacetoxy-6-(acetoxymethyl)tetrahydro-2H-pyran-2-yl)oxy)pentanoic acid

DCM (4.0V), ACN (4.0V), Soft water (6.0V), (2R,3R,4R,5R,6R)-5-acetamido-2-(acetoxymethyl)-6-(hex-5-en-1-yloxy)tetrahydro-2H-pyran-3,4-diyl diacetate (1.0 eq) and RuCl₃—H₂O (0.013 eq) were charged to the reactor and cooled to 0±5° C. NaIO₄ (4.1 eq) was then added to the reactor batch-wise at 0±5° C. and the reaction mixture was stirred for at least 2 hours at 0-5° C. Reaction progress was monitored by HPLC. If the area % of the starting material was >50% after stirring for 8 hours, additional RuCl₃—H₂O (0.001 eq) and NaIO₄ (0.2 eq) was added and the reaction mixture was then stirred for at least 2 hours at 0-5° C. The process was repeated until the area % of the starting material was ≤5% and the reaction mixture through diatomaceous earth (0.5 wt). The pH of the mixture was adjusted to 8 with saturated NaHCO₃ solution and stirred for at least 1 hour at 10±5° C. The mixture was then filtered through diatomaceous earth (0.5 wt), the layers separated, and the aqueous phase collected. The aqueous phase was then extracted with DCM (3.0V×4) and then diluted with DCM (10.0V). The pH of the mixture was adjusted to 1-2 with citric acid at 10±5° C. and stirred for at least 1 hour at 10±5° C. The aqueous phase was then separated and extracted with DCM (5.0V×2). The organic layers were combined and concentrated under vacuum below 40° C. until the system was ≤2.0V. MTBE (4.0V) was charged to the mixture and concentrated until the system was ≤2.0V. MTBE (4.0V) was charged to the mixture and concentrated until the system was ≤3.0V. The mixture was then cooled to 5±5° C., charged with MTBE (3.0V), and stirred for at least 1 hour. The filter cake was centrifuged and rinsed with MTBE (1.0V). The filter cake was dried under vacuum for at least 12 hours at 30±5° C. until LOD ≤5% and the product packaged in double LDPE bags and was stored in well-closed container at −10 to −20° C.

### Example 2. Synthesis of (9H-fluoren-9-yl)methyl (2-(2-hydroxyethoxy)ethyl)carbamate (2)

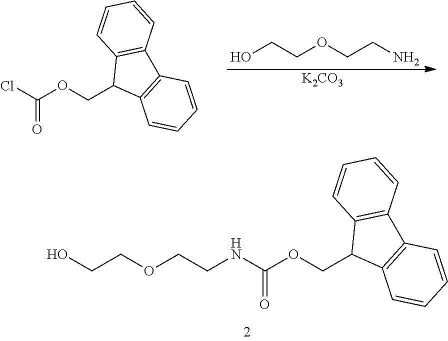

The reactor was vacuumed to ≤−0.08 MPa and then inflated with nitrogen to atmosphere for three times. Water (10V) and K₂CO₃ (2.0 eq.) were charged and stirred for at least 30 mins. The mixture cooled to 5±5° C. and 2-(2-aminoethoxy) ethanol (1.2 eq.) was added. Fmoc-Cl (1.0 eq.) in DCM (5V) was then dropwise at 5±5° C. and afterward warmed to 25±5° C. Reaction progress was monitored by HPLC showing typically Fmoc-Cl ≤1.0% after 10 mins. The layers were separated and the organic phase was washed with water (5.0V×2) and sat. NaCl (5.0V). The organic phase was then concentrated below 35° C. to 2.0V-3.0V. MTBE (3.0V) was added and the organic phase was then concentrated below 35° C. to 2.0V-3.0V. n-Hexane (10.0 v) was then added dropwise for at least 1.5 h and the resulting mixture was stirred for at least 30 mins at 20±5° C. The mixture was then cooled to 10±5° C., centrifuged, and the cake washed with n-hexane (2.0V). The cake was dried under vacuum at 30±5° C. at least 4 hours or until LOD was not more than 5% and KF was not more than 1%. The product was then packaged in double low-density polyethylene bags sealed with cable ties and store in well-closed container at −10 to −20° C.

### Example 3. Synthesis of N-(9-((6aR,8R,9R,9aR)-9-((2-(2-aminoethoxy)ethoxy)methoxy)-2,2,4,4-tetraisopropyltetrahydro-6H-furo[3,2-f][1,3,5,2,4]trioxadisilocin-8-yl)-9H-purin-6-yl)benzamide bifumarate (3)

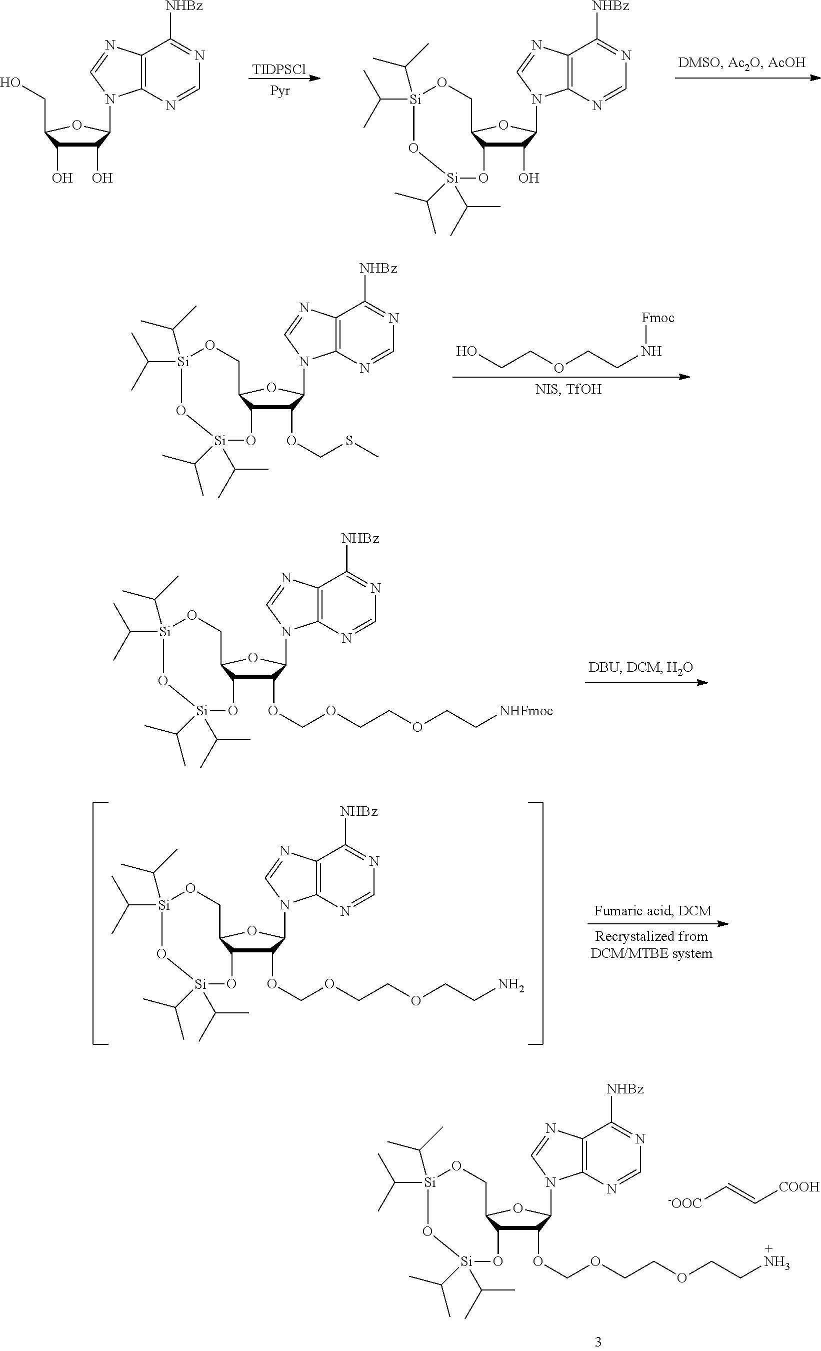

### Step 1: N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide

DMF (3V), pyridine (2V) and N-(9-((2R,3R,4S,5R)-3,4-dihydroxy-5-(hydroxymethyl)tetrahydrofuran-2-yl)-9H-purin-6-yl)benzamide (1.0 eq) were charger into a reactor and warmed to 30±5° C. and stirred for at least 10 mins. The mixture was concentrated below 65° C. to removed water to ≤0.1% using repeated dilutions of acetonitrile (5V/each time to 5±0.5V) determined by KF analysis. The resulting mixture was then cooled to 25±5° C. and supplementary DMF (2V) and Pyridine (1V) was charged. The mixture was further cooled to 10±5° C. and TIDPSCl (1.05 eq) was added dropwise at 5-25° C. The reaction mixture was warmed to 25±5° C. and monitored by HPLC until area % of starting material was ≤3.0% after stirring for at least 3 hours at 25±5° C. Thereafter, EA (10 v) was added to the reaction mixture and cooled to 10±5° C. The reaction was quenched with 20% citric acid (5V) between 5-25° C., charged with sat. NaCl (5V), stir for at least 30 mins, let stand for at least 30 mins, and separated. The organic layer was washed with 20% citric acid (5V) and water (5V×3). The organic phase was then concentrated to 3±0.5V and then solvent swapped to MTBE until the area % of EA was ≤20% by GC. MTBE (2V) was then added and n-heptane (30V) was added dropwise at 20±5° C. in 2 hours, followed by stirring for at least 2 hours at 20±5° C. The mixture was cooled to 10±5° C. and stir for at least 1 hour before centrifuge. The cake was then washed with n-heptane (3V) and dried under vacuum until LOD was not more than 5.0% for at least 8 hours at 30±5° C. The product was then packaged in plastic bag under nitrogen and store at −10 to −20° C.

### Step 2: N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide

DMSO (2.0V) and N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide (1.0 eq) was charged to a reactor at 25±5° C. and cooled to 10±5° C. AcOH (2.0V) was then added dropwise followed by Ac₂O (1.5V) below 25° C. The reaction mixture was then warmed to 30±5° C. for 15 h and monitored by HPLC for reaction completeness. Thereafter, the reaction mixture was diluted with EA (10V) and cooled to 10±5° C. The reaction was quenched with sat. potassium carbonate (7V) between 25±5° C. and stirred for at least 1 h at 25±5° C. The layers were then separated and the organic phase was diluted with water (5V), stirred for at least 30 mins, and separated. The organic phase was concentrated to 2±0.5V and solvent swapped with acetonitrile until the area % of EA was ≤1.0% by GC. Acetonitrile (5V) was then charged and the mixture was warmed to 40±5° C. until the solids dissolved. The solution was stirred for at least 1 hour at 40±5° C., cooled to 30±5° C. and stir for at least for 1 hour, cooled to 20±5° C. and stir for at least for 2 hours, cooled to 10±5° C. and stir for at least 1 hour, centrifuged and the cake was washed with n-heptane (0.5V×2). The cake was dried under vacuum for at least 5 hours at 30±5° C. and the produce was packaged in plastic bag and stored at −10 to −20° C. until slurried. The product, acetonitrile (2.5V), and H₂O (2.5V) were then charged into a reactor and stirred for 30-60 mins at 20±5° C. The mixture was centrifuged and cake washed with ACN:H₂O=1:1 (0.5V). The cake was then dried for at least 8 hours at 30±5° C. and analyzed by HPLC, LOD, and KF. The product was packaged in double low-density polyethylene bags sealed with cable ties and stored in well-closed container at −10 to −20° C.

### Step 3: (9H-fluoren-9-yl)methyl (2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)carbamate

DCM (12.0V), N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide (1.0 eq) and (9H-fluoren-9-yl)methyl (2-(2-hydroxyethoxy)ethyl)carbamate (2, 1.2 eq) were charged into a reaction and stirred to get a clear solution. The solution was then concentrated to 6.5±0.5V, charged with DCM (12.0V), and then concentrated to 11.5±0.5V. 4A Molecular sieve (1.0 wt) were then added and the mixture was stirred for at least 30 mins. The mixture was then cooled to −30±5° C. and charged with NIS (1.2 eq). TfOH (2.0 eq) was added dropwise (T<−20° C.) and mixture was warmed to −20±5° C. Reaction progress with monitored by HPLC. Thereafter, TEA (0.6V) was added dropwise to the reaction (T<−15° C.) and stirred for at least 15 mins. The resulting cake was washed with DCM (5V) and the filtrate was washed with a mixture of sat. NaHCO₃:10% Na₂SO₃ (5V:5V×2), water (5V, ×2) and sat. NaCl (5V), to obtain a solution of the product to be used directly in the next step.

### Step 4: N-(9-((6aR,8R,9R,9aR)-9-((2-(2-aminoethoxy)ethoxy)methoxy)-2,2,4,4-tetraisopropyltetrahydro-6H-furo[3,2-f][1,3,5,2,4]trioxadisilocin-8-yl)-9H-purin-6-yl)benzamide bifumarate (3)

The DCM solution from Step 2 above was diluted with soft water (7.0V) and cooled to 5±5° C. DBU (0.7V) was added and the reaction progress was monitored by HPLC. Thereafter, the mixture was warmed to 20±5° C., the layers separated, and the organic phase collected. The organic phase was then washed with soft water (10V) to obtain a DCM solution of N-(9-((6aR,8R,9R,9aR)-9-((2-(2-aminoethoxy)ethoxy)methoxy)-2,2,4,4-tetraisopropyltetrahydro-6H-furo[3,2-f][1,3,5,2,4]trioxadisilocin-8-yl)-9H-purin-6-yl)benzamide that was cooled to 15±5° C. Fumaric acid (2.2 eq) and 4A molecular sieves (2.0 wt) (in four portions) were then charged at 15±5° C., and the mixture was stirred at least for 1 hour. The mixture was centrifuged and transfer to reactor through micro filter, washing the cake with DCM (2.0V). MTBE (120.0V) was then charged dropwise at 15±5° C. and stirred for at least 10 hours at 15±5° C. The resulting slurry was then centrifuged and the cake was washed with MTBE (2.0 V). The cake was then dried for at least 6 hours at 25±5° C. and analyzed by HPLC, LOD, and QNMR. The product was packaged in double low-density polyethylene bags sealed with cable ties and stored in a well-closed container below −20° C.

### Example 4. Synthesis of (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (4)

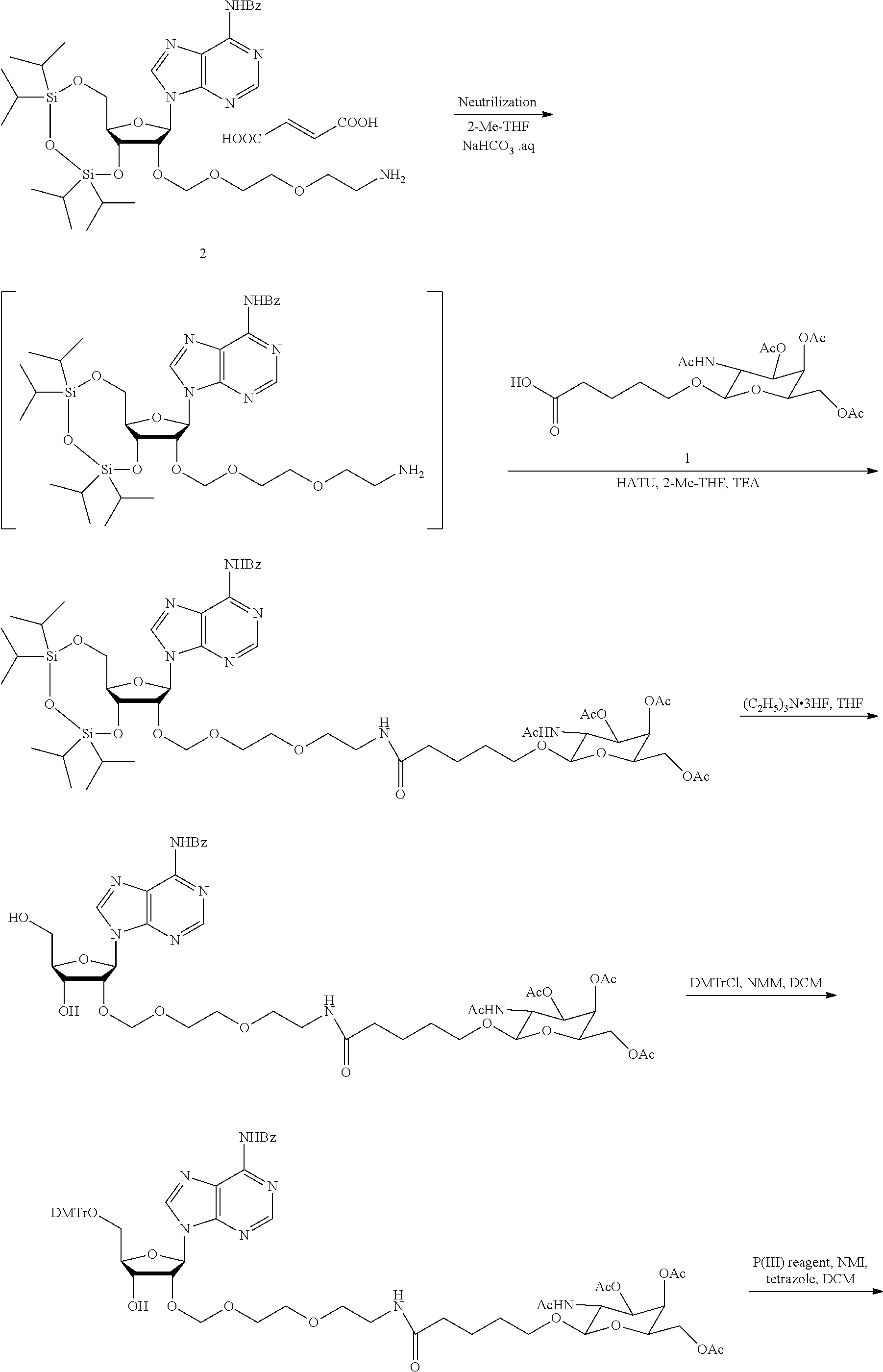

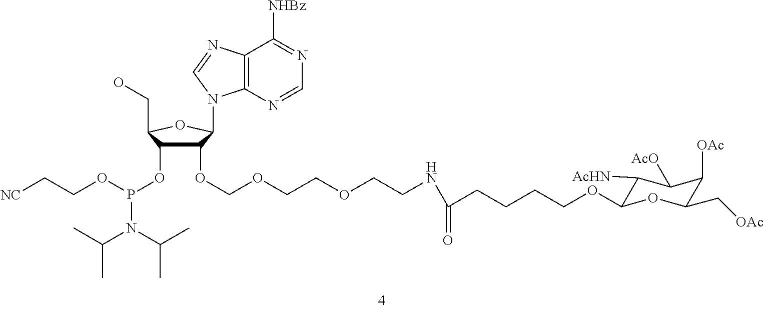

### Step 1: (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate

2-Me-THF (15V) was charged into a reactor, cooled to 0±5° C., and then added N-(9-((6aR,8R,9R,9aR)-9-((2-(2-aminoethoxy)ethoxy)methoxy)-2,2,4,4-tetraisopropyltetrahydro-6H-furo[3,2-f][1,3,5,2,4]trioxadisilocin-8-yl)-9H-purin-6-yl)benzamide bifumarate (3, 1.0 eq). The mixture was then washed with cold aq. NaHCO₃ (4.3%, 10V, ×2), and cold aq. NaCl (20%, 10V, ×3) at 0±5° C., analyzed by HPLC, and the resulting 2-Me-THF solution was cooled to 0±5° C. and charged with 5-(((2R,3R,4R,5R,6R)-3-acetamido-4,5-diacetoxy-6-(acetoxymethyl)tetrahydro-2H-pyran-2-yl)oxy)pentanoic acid (1, 1.1 eq), TEA (3.0 eq), and HATU (1.5 eq) at −5 to 15° C. The mixture was then warmed to 25±5° C. for at least 1 hour with HPLC monitoring. Thereafter, the mixture was allowed to stand for at least 0.5 h, the layers separates, the organic phase was washed with 5% NaCl solution (10V, ×2) and sat. NaCl (10V) at 25±5° C., allowing stirring and siting for at least 0.5 h every time. The organic layer was then separated and concentrated to 3.0V using azeotropic distillation to control water content (≤1.0%).

### Step 2: (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate

The product solution of Step 1 above was charge with THE (5.0V), TEA (3.0 eq), and then charged dropwise with TEA-3HF (3.0 eq) at 10±5° C. The mixture was then warmed to 25±5° C. and monitored after 2 h by HPLC. Thereafter, the mixture was concentrated and solvent swapped with DCM (5V, ×3). The resulting solution was concentrated to 3V and charge with DCM (8V). Sat. NaHCO₃ (10.0 v) was then added dropwise at 10±5° C. The layers were separated and the organic layer washed with soft water (5.0V). The aqueous phase was extracted with DCM (5.0V) and the organic phases were combined and washed with sat. NaCl solution (5.0V). The organic phase was then concentrated to ≤5.0V, added dichloromethane (5.0 v), and concentrated to ≤5.0 v, and then repeat three times. The resulting solution was used directly in the next step.

### Step 3: (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate

The product from step 2 above in DCM was cooled to 10-15° C. and charged with NMM (4.0 eq) below 25° C. and then charged with DMTr-Cl (1.4 eq) in four portions below 25° C. and monitored after 1 h at 25±5° C. by HPLC. Thereafter, the reaction mixture was washed with sat. NaHCO₃ solution (5.0V), soft water (5.0V) and sat. NaCl solution (5.0V). After standing for at least 30 mins and stirring for at least 30 mins the organic phase was concentrated to 3.0±0.5V and purified by Flash-Prep-HPLC with the following conditions: DCM:n-heptane=1:1 (5% TEA) to remove DMTrOH; and then elute with 20% to 80% acetone in n-heptane (5% TEA). The purified fraction was collected and concentrated. EA (5V, 5% TEA) was charged and concentrated to 2.5-3.5V, twice. The resulting concentrated solution was then added dropwise to a solution of 5:1 n-heptane:MTBE (15V, 5% TEA) at 10±5° C. The mixture was then stirred for at least 1 hour at 10±5° C. and then centrifuged. The wet cake was rinsed with n-heptane (2V), dried under vacuum at 35±5° C., and analyzed by LOD, HPCL, and Ru residual test. The product was packaged in double LDPE bags sealed with cable ties and stored in well-closed container at −20±5° C.

### Step 4: (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate

DCM (10 V), the product of step 2 above (1.0 eq), and NMI (1.0 eq) were charged into a reactor. Water was removed azeotropically with DCM by concentrating to 6V and charging 4.0V DCM repeatedly until the content of water was ≤0.05%. The mixture was then cooled to 0±5° C. and the reactor was flushed with nitrogen. Tetrazole (0.5 eq) was then added under nitrogen atmosphere at 0±5° C. followed buy the P-reagent (1.2 eq) under nitrogen atmosphere at 0±5° C. The reaction mixture was then warmed to 25±3° C. and reaction progress was monitored by HPLC (≤1.0% starting material after 2 hours). The mixture was then washed with sat. NaHCO₃ (5V), H₂O (8V), sat. NaCl (5V) and dried with Na₂SO₄ (2.0 wt) with stirring for at least 30 mins. The resulting solution was centrifuged and the cake with washed EA (3V). The filtrate was transferred into a reactor through nutsche filter and concentrated to ≤3.0V, charged with 5.0V EA (5% TEA), concentrated to ≤3.0V, charged with 5.0V EA (5% TEA), and concentrated to 4.0-5.0V. 1^{St} Solidification: Stir the mixture for 30 mins and added dropwise a solution of 5% TEA in 2:3 MTBE:n-heptane (32V, remove oxygen) at 10±5° C., stirred for 30 mins and centrifuged, and wash cake with mixture solution of 2:3 MTBE:n-heptane (4V, 5%, TEA). 2^{nd} Solidification: Cake was completely dissolved in EA (4V, 5% TEA) and added dropwise a solution of 5% TEA in 2:3 MTBE:n-heptane (32V, remove oxygen) at 10±5° C., stirred for 30 mins and centrifuged, and cake was washed with a solution of 2:3 MTBE:n-heptane (4V, 5% TEA). 3^{rd} Solidification: Cake was completely dissolved in EA (4V, 0.5% TEA) and added dropwise a solution of 5% TEA in 2:3 MTBE:n-heptane (32V, remove oxygen) at 10±5° C., stirred for 30 mins and centrifuged, and then the cake was washed with a mixture solution of 2:3 MTBE:n-heptane (4V, 5% TEA). Product cake was analyzed by HPLC and P-NMR and dried under vacuum for at least 12 hours at 35±5° C. and further analyzed for particulates, GC, and KF. The product was then packaged in an HDPE bottle and then heat sealed in aluminum foil bag with outer fiber keg, and then stored at −15 to −25° C.

### Example 6. Post-Synthetic Conjugation of GalNAc to Adem-Amine Linker (G, a, C, U) of a GalXC Derivative

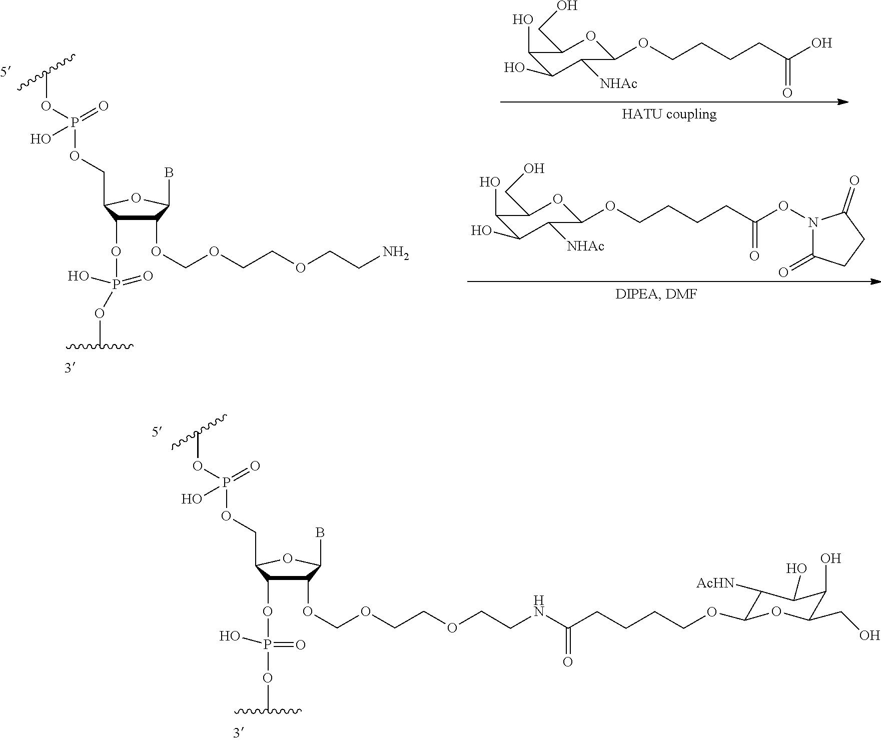

1. HATU Coupling

In a 15 mL falcon tube, the sense strand of a GalXC type construct with four adem-amine linkers is dissolved in water (1 eq) and then diluted with DSMO. In a separate 1.5 mL Eppendorf vial, the GalNAc-acid (13.2 eq) is dissolved in anhydrous DMSO (150 μL). To this solution containing the GalNAc acid, HATU ((1-[Bis(dimethylamino)methylene]-1H-1,2,3-triazolo[4,5-b]pyridinium 3-oxidi hexafluorophosphate, 13.2 eq) in DMSO (50 μL) and N, N-Diisopropylethylamine (9.4 μl, 27.0 eq) were added. After 5 minutes, the solution containing the sense strand was added to the reaction mixture. The reaction mixture was placed in a shaker and monitored by UPLC-MS for desired product formation. The reaction mixture was purified by ion-pairing chromatography (Water/Acetonitrile containing 100 mM triethylammonium acetate). The product fractions were pooled and dialyzed against water 3× using a 15 mL Millipore 10K membrane and lyophilized in a 15 mL Falcon tube to afford an amorphous white solid. The sense strand can then be annealed to the corresponding antisense strand using established procedures to afford a solution of a tetra-GalNAc conjugated DsiRNA duplex. Equivalents of reagents can be altered depending on the number of desired GalNAc moieties introduced to the sense strand.

2. NHS Ester Coupling

In a 1.5 mL Eppendorf vial, the GalNAc NHS ester (13.2 eq) was dissolved in anhydrous DMSO (200 μL). In a separate 15 mL falcon tube, the sense strand of a GalXC type construct with four adem-amine linkers (1 eq) was dissolved in water (2000 μL) and diluted with DMSO (200 μL). The solution containing the GalNAc NHS ester was added to the solution containing the sense strand followed by the addition of triethylamine (30.67 μL). The resulting solution was placed in a shaker and monitored by UPLC-MS for desired product formation. The reaction mixture was purified by ion-pairing chromatography (Water/Acetonitrile containing 100 mM triethylammonium acetate. The product fractions were pooled and dialyzed against water 3× using a 15 mL Millipore 10K membrane and lyophilized in a 15 mL Falcon tube to afford an amorphous white solid. The sense strand can then be annealed to the corresponding antisense strand using established procedures to afford a solution of a tetra-GalNAc conjugated DsiRNA duplex. Equivalents of reagents can be altered depending on the number of desired GalNAc moieties introduced to the sense strand.

### Example 7. Salt Screen of Intermediate

Intermediate compound N-(9-((6aR,8R,9R,9aR)-9-((2-(2-aminoethoxy)ethoxy)methoxy)-2,2,4,4-tetraisopropyltetrahydro-6H-furo[3,2-f][1,3,5,2,4]trioxadisilocin-8-yl)-9H-purin-6-yl)benzamide is unstable. In order to shorten GMP steps and to simplify post-processing operation, a salt screen was performed with this intermediate compound. Acid was dissolved in acetone and added dropwise to a solution of the intermediate compound in DCM. Results using certain exemplary acids are shown in Table 2.

TABLE 2
Salt screen

| Acid | Result |
|---|---|
| L-DBTA (1.1 eq) | Solid appeared |
| Citric acid (1.1 eq) | — |
| pTSA (1.1 eq) | — |
| Fumaric acid (1.1 eq) | Solid appeared |
| Conc. Sulfuric acid (1.1 eq) | — |
| Oxalic acid (1.1 eq) | — |
| (+)-L-Tartaric acid (1.1 eq) | Solid appeared |
| (−)-L-Malic acid ((1.1 eq) | — |
| 2M HCl in MTBE (l.1 eq) | — |

After extensive screening of a number of acids and conditions, it was found that fumaric acid salt of the intermediate compound was stable and could be isolated. After further experimentation altering the equivalents of fumaric acid, bifumarate salt of the intermediate was found to provide desired properties, including reduced solvent volume needed for solidification.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method for preparing a fragment compound of formula F-4-a: 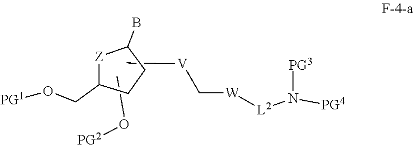
or a pharmaceutically acceptable salt thereof, wherein:
PG¹ and PG² are independently hydrogen or a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 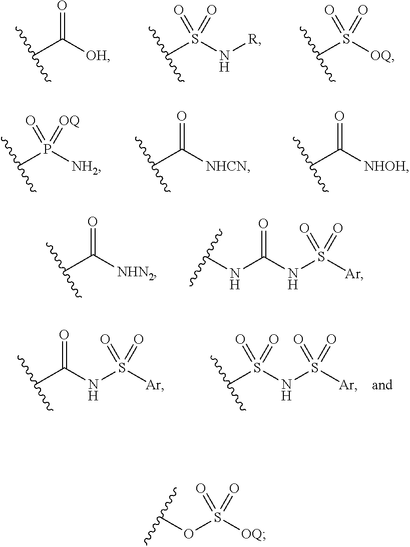
each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—,
comprising the steps of:
(a) providing a fragment compound of formula F-1-a: 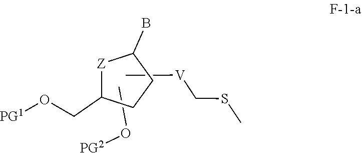
or a pharmaceutically acceptable salt thereof, and
(b) alkylating said compound with a compound of formula F-2: 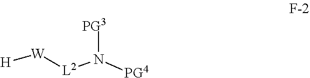
or a pharmaceutically acceptable salt thereof, to form a fragment compound of formula F-4-a.

2. The method according to claim 1, further comprising the step of preparing a compound of formula F-5-a: 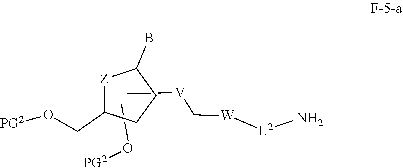
or a pharmaceutically acceptable salt thereof, wherein:
PG¹ and PG² are independently hydrogen or a suitable hydroxyl protecting group;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 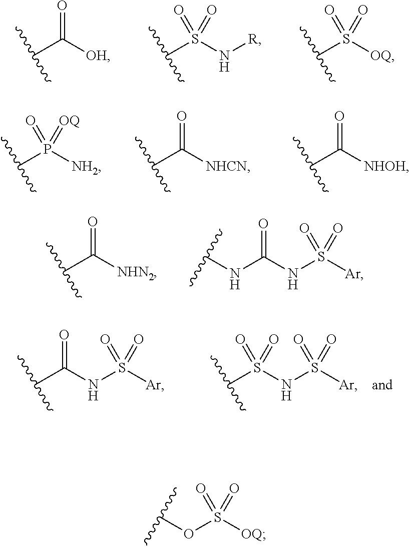
each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—,
comprising the steps of:
(a) providing a compound of formula F-4-a: 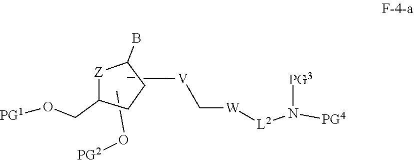
or a pharmaceutically acceptable salt thereof, and
(b) deprotecting said fragment compound of formula F-4-a to form the fragment compound of formula F-5-a.

3. The method of claim 2, further comprising the steps of preparing a compound of formula D-a: 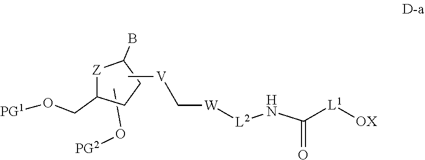
or a pharmaceutically acceptable salt thereof, wherein:
PG¹ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 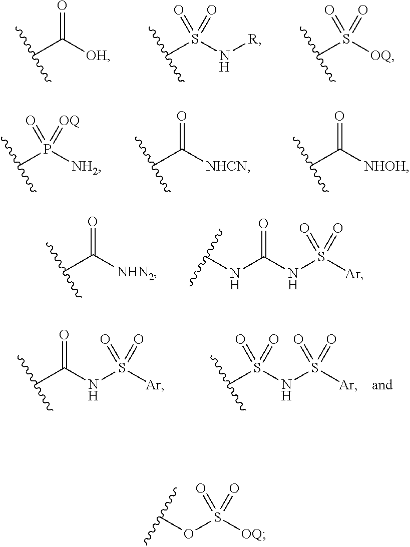
each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and 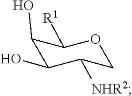
R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
comprising the steps of:
(a) providing a compound of formula F-3: 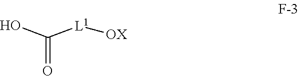
or a pharmaceutically acceptable salt thereof, and
(b) reacting said fragment compound of formula F-3 with a fragment compound of formula F-5-a: 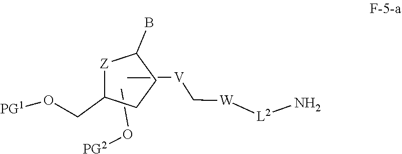
or a pharmaceutically acceptable salt thereof, to provide the compound of formula D-a.

4. A method for preparing a compound of formula D-a: 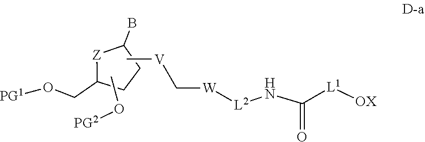
or a salt thereof, wherein:
PG¹ and PG² are independently hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 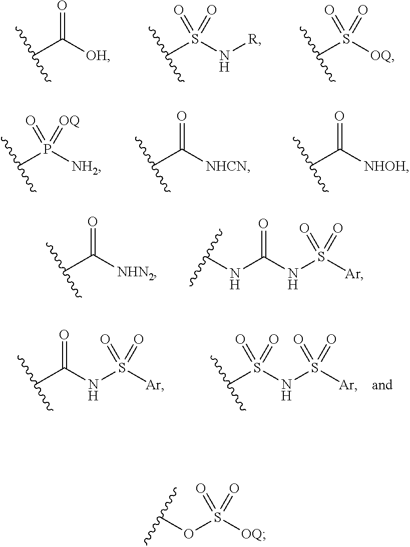
each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and 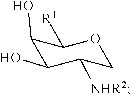
R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
comprising the steps of:
(a) providing a compound of formula F-1-a: 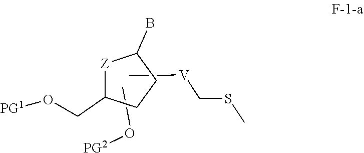
or a salt thereof, and
(b) reacting said fragment compound of formula F-1-a with a fragment compound of formula F-6: 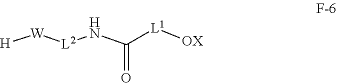
or a salt thereof,
to provide the compound of formula D-a.

5. The method any one of claims 3-4, further comprising the step of preparing a compound of formula C-a: 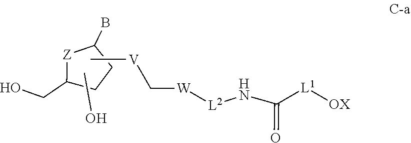
or a pharmaceutically acceptable salt thereof, wherein:
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 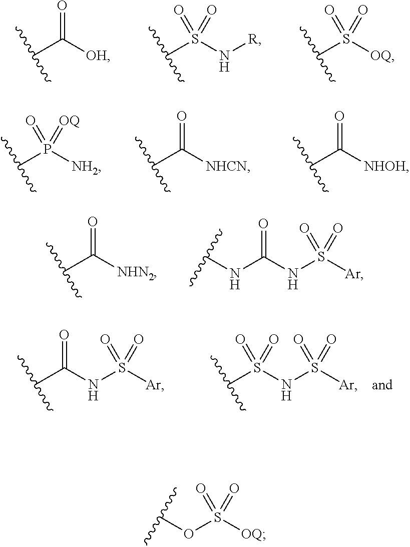
each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and 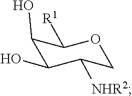
R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
comprising the steps of:
(a) providing a compound of formula D-a: 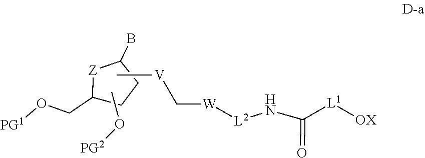
or a pharmaceutically acceptable salt thereof, and
(b) deprotecting said compound of formula D-a to form a compound of formula C-a.

6. The method according to claim 5, further comprising the step of preparing a compound of formula B-a: 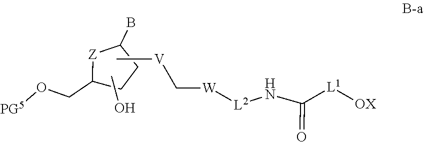
or a pharmaceutically acceptable salt thereof, wherein:
PG⁵ is hydrogen or a suitable hydroxyl protecting group;
B is a nucleobase or hydrogen;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 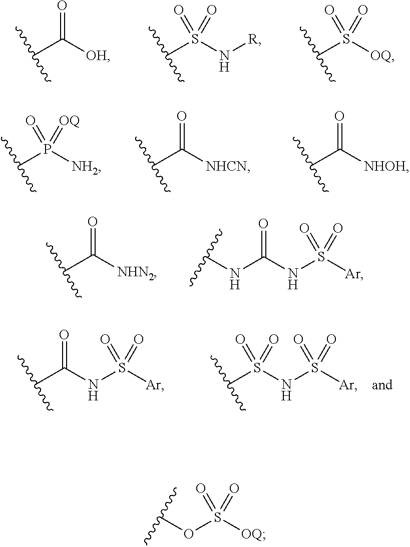
each R is independently selected from hydrogen, alkyl, alkenyl, aromatic, heterocycle, substituted alkyl, and substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and 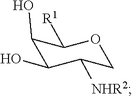
R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
comprising the steps of:
(a) providing a compound of formula C-a: 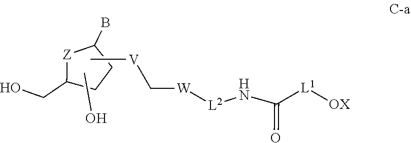
or a pharmaceutically acceptable salt thereof, and
(b) protecting said compound of formula C-a with a suitable protecting group to form a compound of formula B-a.

7. The method of claim 6, further comprising the steps of preparing a compound of formula A-a: 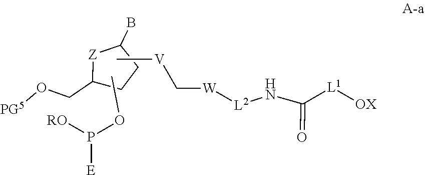
or a pharmaceutically acceptable salt thereof, wherein:
PG⁵ is hydrogen or a suitable hydroxyl protecting group;
B is a nucleobase or hydrogen;
E is a halogen or NR₂;
each L¹ and L² are independently a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 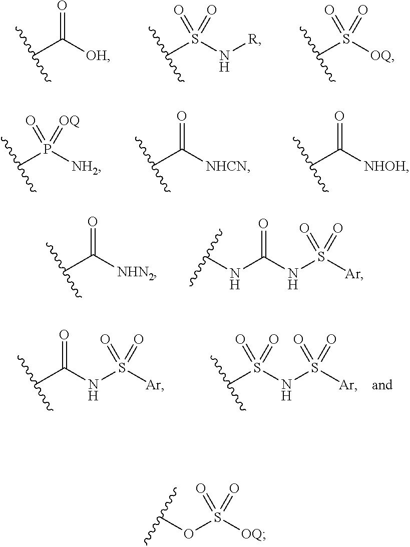
each R is independently selected from hydrogen, alkyl, alkenyl, aromatic, heterocycle, substituted alkyl, and substituted alkenyl, or:
two R groups on the same nitrogen are optionally taken together with their intervening atoms to form a 4-7 membered saturated or partially unsaturated heterocyclic ring having 0-3 heteroatoms, in addition to the nitrogen, independently selected from nitrogen, oxygen, and sulfur;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—;
X is a ligand selected from GalNAc, D-mannose, L-galactose, D-arabinose, L-fucose, polyols, and 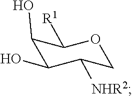
R¹ is selected from CF₃, alkyl, alkenyl, alkynyl, aromatic, heterocycle, substituted alkyl, substituted alkenyl, and substituted alkynyl;
R² is selected from one or more methylenes interrupted or terminated by one or more of P(O)H, P(O₂), P(O₄), polyethylenegylcol (PEG), OR³, S, S(OR³), SO₂(R³), (C═O)OR³, NY₂, NH, and NH(C═OR³);
R³ is H, C₁-C₆ alkanyl, C₁-C₆ alkenyl, or aryl; and
Z is —CH₂—, —O—, —S—, or —NR—,
comprising the steps of:
(a) providing a compound of formula B-a: 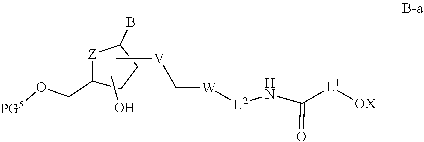
or a pharmaceutically acceptable salt thereof, and
(b) reacting said compound of formula B-a with a P(III) forming reagent to form a compound of formula A-a.

8. The method of claim 7, wherein E is NR₂.

9. The method of claim 8, wherein R is selected from isopropyl and

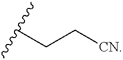 [SMILES: *CCC#N]

10. The method of claim 1, wherein PG³ is H and PG⁴ is Fmoc.

11. The method of any one of claims 1-4, wherein PG¹ and PG² are taken together with their intervening atoms to form a cyclic diol protecting group.

12. The method of claim 11, wherein the cyclic diol protecting group comprises 1,1,3,3-tetraisopropylidisiloxanylidene.

13. The method of any one of claims 6-7, wherein PG⁵ is 4,4′-dimethyoxytrityl.

14. The method of any one of claims 1-13, wherein B is a purine or pyrimidine base.

15. The method of claim 14, wherein the purine or pyrimidine base is G, A, or C comprising a protecting group.

16. The method of claim 14, wherein purine or pyrimidine base is selected from

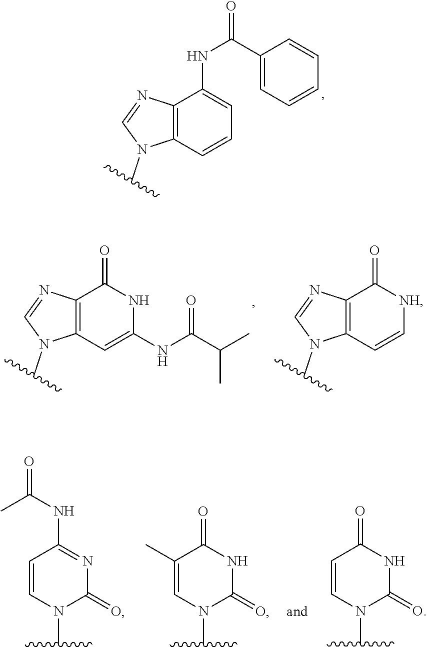

17. The method of any one of claims 1-16, wherein V is —O—.

18. The method of any one of claims 1-17, wherein W is —O—.

19. The method of any one of claims 1-18, wherein Z is —O—.

20. A compound of formula F-4-a: 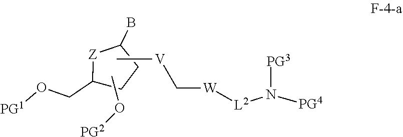
or a pharmaceutically acceptable salt thereof, wherein:
PG¹ and PG² are independently hydrogen or a suitable hydroxyl protecting group;
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 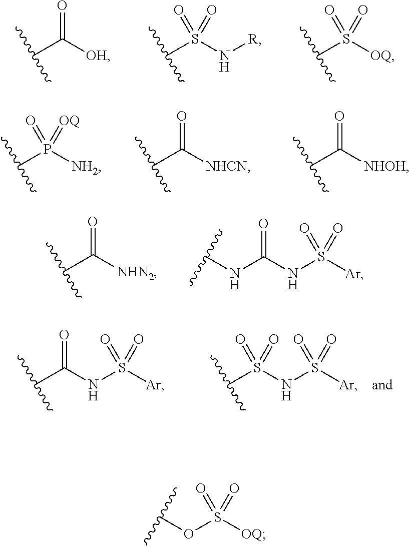
each R is independently selected from hydrogen, alkyl, alkenyl, aromatic, heterocycle, substituted alkyl, substituted alkenyl;
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

21. The compound of claim 20, wherein PG³ is H and PG⁴ is Fmoc or trifluoroacetyl.

22. The compound of any one of claims 20-21, wherein PG¹ and PG² are taken together with their intervening atoms to form a cyclic diol protecting group.

23. The compound of claim 23, wherein the cyclic diol protecting group comprises 1,1,3,3-tetraisopropylidisiloxanylidene.

24. The compound of any one of claims 20-24, wherein B is a purine or pyrimidine base.

25. The method of claim 24, wherein the purine or pyrimidine base is G, A, or C comprising a protecting group.

26. The compound of claim 24, wherein purine or pyrimidine base is selected from

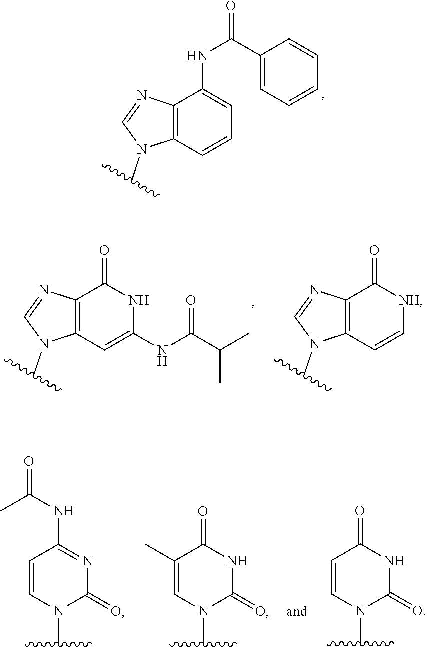

27. The compound of any one of claims 20-26, wherein V is —O—.

28. The compound of any one of claims 20-27, wherein W is —O—.

29. The compound of any one of claims 20-28, wherein Z is —O—.

30. A nucleic acid or analogue thereof compound P2-a, or a pharmaceutically acceptable salt thereof, comprising: 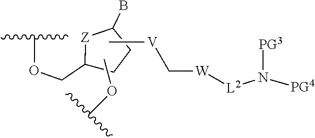
wherein
PG³ and PG⁴ are independently hydrogen or a suitable nitrogen protecting group, provided both PG³ and PG⁴ are not hydrogen at the same time;
B is a nucleobase or hydrogen;
L² is a bivalent moiety selected from alkyl, alkenyl, alkynyl, aromatic, heterocycle, substituted alkyl, substituted alkenyl, or substituted alkynyl, wherein one or more methylenes can be interrupted or terminated by one or more of P(O)H, P(O₂), P(O₄), polyethylenegylcol (PEG), OY, S, S(OY), SO₂(Y), (C═O)OY, NY₂, NH, and NH—(C═OY);
Y is independently selected from H, C₁-C₆ alkanyl, C₁-C₆ alkenyl or aryl, including 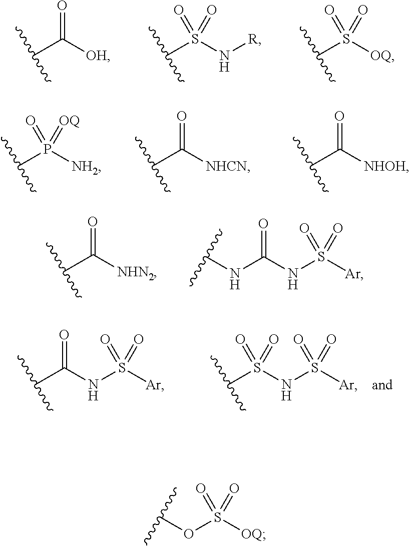
each R is independently selected from hydrogen, alkyl, alkenyl, aromatic, heterocycle, substituted alkyl, and substituted alkenyl, or:
Q is H or a pharmaceutically acceptable salt, C₁-C₆ alkanyl, C₁-C₆ alkenyl, C₁-C₆ alkynyl, aryl, heteroaryl, (CH₂)ₘ-aryl or (CH₂)ₘ-heteroaryl where m is 1-10 and any of the aryl or heteroaryl rings may be substituted with one to three independently selected Cl, F, CF₃, C₁-C₈ alkoxy, NO₂, C₁-C₆ alkanyl, C₁-C₆ alkenyl, aryl or OY, C(O)OY, NY₂ or C(O)NHY;
V and W are independently —O—, —S—, or —NR—; and
Z is —CH₂—, —O—, —S—, or —NR—.

31. The compound of claim 30, wherein PG³ is H and PG⁴ is Fmoc or trifluoroacetyl.

32. The compound of any one of claims 30-31, wherein B is a purine or pyrimidine base.

33. The method of claim 32, wherein the purine or pyrimidine base is G, A, or C comprising a protecting group.

34. The compound of claim 32, wherein purine or pyrimidine base is selected from

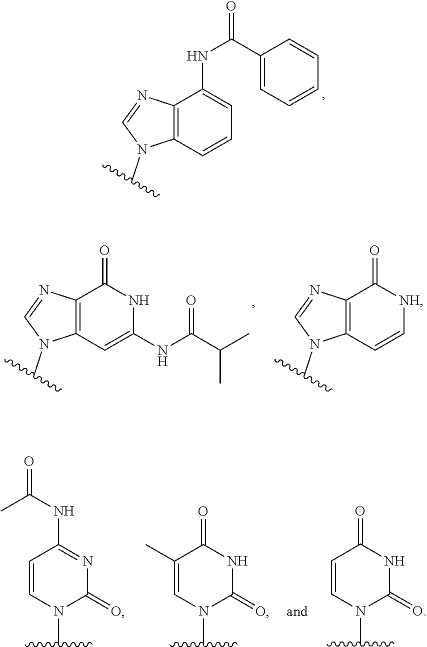

35. The compound of any one of claims 30-34, wherein V is —O—.

36. The compound of any one of claims 30-35, wherein W is —O—.

37. The compound of any one of claims 30-36, wherein Z is —O—.
